(19) United States
(12) Patent Application Publication
(10) Pub. No.: US 2026/0253667 A1
(43) Pub. Date: Aug. 27, 2026

(54) METHODS FOR EVALUATING CLONAL TUMOR MUTATIONAL BURDEN

(71) Applicant: Foundation Medicine, Inc., Boston, MA (US)

(72) Inventors: Karthikeyan MURUGESAN, Boston, MA (US); Zainab ARSHAD, Atlanta, GA (US)

(73) Assignee: Foundation Medicine, Inc., Boston, MA (US)

(21) Appl. No.: 19/107,533

(22) PCT Filed: Aug. 30, 2023
(86) PCT No.: PCT/US2023/073190

§ 371 Date: Feb. 27, 2025

Related U.S. Application Data
(60) Provisional application No. 63/402,890, filed on Aug. 31, 2022.

(51) Int. Cl. G16B 20/20; C12Q 1/6855; C12Q 1/6886

(52) U.S. Cl. CPC G16B 20/20; C12Q 1/6855; C12Q 1/6886; C12Q 2600/156

(57) ABSTRACT

Methods of determining clonal tumor mutational burden (cTMB) in a sample, e.g., a tumor sample or a sample derived from a tumor, from a subject, are disclosed. In some aspects, methods of using the determined cTMB are disclosed.

### CROSS-REFERENCE TO RELATED APPLICATIONS

This application claims the priority benefit of U.S. Provisional Patent Application No. 63/402,890, filed Aug. 31, 2022, the entire contents of which are incorporated herein by reference for all purposes.

### FIELD OF THE INVENTION

The present disclosure relates generally to methods of evaluating gene alterations (e.g., complex genomic biomarkers) such as clonal and subclonal tumor mutational burden, as well as treating patients and selecting treatments for patients based on a clonal or subclonal tumor mutational burden.

### BACKGROUND

Cancer cells accumulate mutations during cancer development and progression. These mutations may be the consequence of intrinsic malfunction of DNA repair, replication, or modification, or exposures to external mutagens. Certain mutations have conferred growth advantages on cancer cells and are positively selected in the microenvironment of the tissue in which the cancer arises. While the selection of advantageous mutations contributes to tumorigenesis, the likelihood of generating tumor neoantigens also increase as mutations develop (Gubin et al., CANCER. The odds of immunotherapy success, Science, vol. 350, no. 6257, pp. 158-159 (2015)).

High tumor mutational burden (TMB-H) is associated with a high neoantigen burden and a subsequent presence of activated effector T cells that results in enhanced responsiveness to immune checkpoint inhibition, e.g., Snyder et al., Genetic basis for clinical response to CTLA-4 blockade in melanoma, NEJM, vol. 371, no. 23, pp. 2189-2199 (2014) and McGranahan et al., Tumor Heterogeneity Correlates with Less Immune Response and Worse Survival in Breast Cancer Patients, Science, vol. 351, no. 6280, pp. 1463-1469 (2016). Subsequently, TMB-H, which is defined as having a TMB of at least 10 mutations per megabase, has been approved by the FDA as a biomarker of immune checkpoint inhibitor response for metastatic solid tumors.

### BRIEF SUMMARY OF THE INVENTION

Disclosed herein are methods and systems for identifying and determining a clonal tumor mutational burden (cTMB) for the sample based on the number of cTMB-qualified short variants. In some embodiments, the method and systems comprise determining cTMB-qualified variants by filtering the short variants for one or more tumor amount measure, for example, cancer cell fraction (CCF) of the variant.

Provided herein is a method comprising obtaining, using one or more processors, a plurality of sequence read data that represent a set of nucleic acid molecules obtained from a sample for a subject; identifying, using the one or more processors, one or more short variants from the sequence read data; filtering, using the one or more processors, the one or more short variants to select one or more clonal tumor mutational burden (cTMB)-qualified short variants, wherein the filtering comprises excluding short variants identified as subclonal based on a tumor amount measure for a respective short variant; and determining, using the one or more processors, a cTMB value (for example, a cTMB score) for the sample based on the number of cTMB-qualified short variants.

Also provided herein is a method comprising receiving, at one or more processors, genomic data for a subject, wherein the genomic data indicates a presence or absence of one or more short variants in a sample derived from the subject; filtering, using the one or more processors, the one or more short variants to select one or more clonal tumor mutational burden (cTMB)-qualified short variants, wherein the filtering comprises excluding short variants identified as subclonal based on a tumor amount measure for a respective short variant; and determining, using the one or more processors, a cTMB value (for example, a cTMB score) for the sample based on the number of cTMB-qualified short variants.

In any of the provided embodiments, the subject has a cancer.

In any of the provided embodiments, the cTMB is determined with one or more of (a) counting a number of the one or more clonal short variants in the sample, and (b) normalizing a size of a bait molecule. In any of the provided embodiments, the tumor amount measure comprises a cancer cell fraction (CCF) of the short variant or a variant allele frequency (VAF). In any of the provided embodiments, the filtering comprises excluding short variants identified as having an allele frequency below a predetermined threshold. In any of the provided embodiments, the filtering comprises excluding short variants identified as a germline variant. In any of the provided embodiments, the filtering comprises excluding short variants identified as having a cancer cell fraction (CCF) at or below a predetermined CCF threshold.

In any of the provided embodiments, a clonal tumor mutational burden (cTMB) value (for example, a cTMB score) is expressed as a function of the number of cTMB-qualified short variants per megabase (mutations/Mb) in a set of subgenomic intervals from the sample. In some embodiments, the set of subgenomic intervals from the sample are between about 100 kb to about 10 Mb. In some embodiments, the set of subgenomic intervals from the sample are between about 0.8 Mb to about 1.1 Mb.

In any of the provided embodiments, the genomic data for the subject is based on a targeted exome sequencing panel. In any of the provided embodiments, the genomic data for the subject is derived from a single biopsy sample. In any of the provided embodiments, the genomic data for the subject is derived from only one biopsy sample. In any of the provided embodiments, the genomic data for the subject is derived from circulating tumor DNA in a liquid biopsy sample. In any of the provided embodiments, the genomic data for the subject is derived from single cell sequencing.

In any of the provided embodiments, the one or more short variants include noncoding and synonymous short variants.

In any of the provided embodiments, the method further comprises obtaining the sample from the subject. In any of the provided embodiments, the sample comprises a tissue biopsy sample, a liquid biopsy sample, or a normal control. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof.

In any of the provided embodiments, the plurality of nucleic acid molecules comprises a mixture of tumor nucleic acid molecules and non-tumor nucleic acid molecules. In some embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In some embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In any of the provided embodiments, the genomic data is obtained from sequencing the sample. In some embodiments, the sequencing comprises use of a massively parallel sequencing (MPS) technique, whole genome sequencing (WGS), whole exome sequencing, targeted sequencing, direct sequencing, or Sanger sequencing technique. In some embodiments, the sequencing comprises massively parallel sequencing, and the massively parallel sequencing technique comprises next generation sequencing (NGS). In some embodiments, the sequencing comprises providing a plurality of nucleic acid molecules obtained from the sample from the subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the set of nucleic acid molecules in the sample.

In some aspects, provided herein is a method where the one or more adapters comprise amplification primers, flow cell adapter sequences, unique molecular identifier sequence, substrate adapter sequences, or sample index sequences. In some embodiments, the captured nucleic acid molecules are captured from the amplified nucleic acid molecules by hybridization to one or more bait molecules. In some embodiments, the one or more bait molecules comprise one or more nucleic acid molecules, each comprising a region that is complementary to a region of a captured nucleic acid molecule. In some embodiments, amplifying nucleic acid molecules comprises performing a polymerase chain reaction (PCR) amplification technique, a non-PCR amplification technique, or an isothermal amplification technique.

In some aspects, the plurality of sequence reads overlap one or more gene loci within a subgenomic interval in the sample. In any of the provided embodiments, the one or more short variants comprise ALOX12B T402T, ATRX K2225del, CTNNA1 D362N, FLT3 S102S, JAK2 1289V, KEAP1 K97N, KRAS Q61H, MSH3 R573fs*4, PDK1 T306M, STAG2 C527F, STK11 649_650delCC, TSC2 A68S, or any combination thereof. In any of the provided embodiments, the one or more cTMB-qualified short variants comprise CTNNA1 D362N, FLT3 S102S, STAG2 C527F, TSC2 A68S, or any combination thereof.

In some aspects, provided herein is a method further comprising generating, by the one or more processors, a report comprising a cTMB for the subject. In some embodiments, comprising transmitting the report to a healthcare provider. In some embodiments, the report is transmitted via a computer network or a peer-to-peer connection.

Also provided herein is a method of treating a subject having a cancer comprising (a) determining a clonal tumor mutational burden (cTMB) value (for example, a cTMB score) in a sample from the subject by the method of some of the previous embodiments; and (b) treating the subject with an immuno-oncology (IO) therapy if the cTMB value determined for the sample is at or above a threshold cTMB value (or threshold cTMB score).

Also provided herein is a method of selecting a treatment for a subject having a cancer, the method comprising determining a clonal tumor mutational burden (cTMB) value (for example, a cTMB score) in a sample from the subject by the method of some of the previous embodiments, wherein if the cTMB value determined for the sample is at or above a threshold cTMB value (or cTMB score), the subject is identified as one who may benefit from treatment with an immune-oncology (IO) therapy.

Also provided herein is a method of identifying a subject having a cancer for treatment with an immune-oncology (IO) therapy comprising (a) determining a clonal tumor mutational burden (cTMB) value (for example, a cTMB score) in a sample from the subject by the method of some of the previous embodiments; and (b) identifying the subject for treatment with the IO therapy if the cTMB value determined for the sample is at or above a threshold cTMB value (or threshold cTMB score).

Also provided herein is a method of identifying one or more treatment options for a subject having a cancer, the method comprising (a) determining a clonal tumor mutational burden (cTMB) value (for example, a cTMB score) in a sample from the subject by the method of some of the previous embodiments; and (b) generating a report comprising one or more treatment options identified for the subject based at least in part on the cTMB value determined for the sample, wherein if the cTMB value is at or above a threshold cTMB value (or threshold cTMB score), the subject is identified as one who may benefit from treatment with an immune-oncology (IO) therapy.

Also provided herein is a method of predicting survival of a subject having cancer, the method comprising determining a clonal tumor mutational burden (cTMB) value (for example, a cTMB score) in a sample from the subject by the method of some of the previous embodiments, wherein if the cTMB value determined for the sample is at or above a threshold cTMB value (or threshold cTMB score), the subject is predicted to have longer survival when treated with an immune-oncology (IO) therapy, as compared to a subject determined to have a cTMB value below the threshold cTMB value.

In some aspects, provided herein is a method where the threshold cTMB value (or threshold cTMB score) is at least about 4 to 100 mutations/Mb, about 4 to 30 mutations/Mb, 8 to 100 mutations/Mb, 8 to 30 mutations/Mb, 10 to 20 mutations/Mb, less than 4 mutations/Mb, or less than 8 mutations/Mb. In some embodiments, the threshold cTMB value is at least about 5 mutations/Mb, at least about 10 mutations/Mb, at least about 12 mutations/Mb, at least about 16 mutations/Mb, at least about 20 mutations/Mb, or at least about 30 mutations/Mb. In some embodiments, the threshold cTMB value (or threshold cTMB score) is at least about 7.5 mutations/Mb, at least about 8.5 mutations/Mb, at least about 9.5 mutations/Mb, at least about 11 mutations/Mb, at least about 13.5 mutations/Mb, at least about 16.5 mutations/Mb, at least about 18.5 mutations/Mb, at least about 19 mutations/Mb, or at least about 25 mutations/Mb.

In some aspects, the immuno-oncology (IO) therapy comprises a single IO agent or multiple IO agents. In some embodiments, the immuno-oncology (IO) therapy comprises an immune checkpoint inhibitor. In some embodiments, the immune checkpoint inhibitor comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a cellular therapy, a treatment for cancer being tested in a clinical trial, an immunotherapy, or any combination thereof.

In some aspects, the immune checkpoint inhibitor is a PD-1 inhibitor. In some embodiments, the immune checkpoint inhibitor comprises one or more of nivolumab, pembrolizumab, cemiplimab, or dostarlimab. In some embodiments, the immune checkpoint inhibitor is a PD-L1-inhibitor. In some embodiments, the immune checkpoint inhibitor comprises one or more of atezolizumab, avelumab, or durvalumab. In some embodiments, the immune checkpoint inhibitor is a CTLA-4 inhibitor. In some embodiments, the CTLA-4 inhibitor comprises ipilimumab. In some embodiments, the nucleic acid comprises a double-stranded RNA (dsRNA), a small interfering RNA (siRNA), or a small hairpin RNA (shRNA). In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In some aspects, provided herein is a method further comprising treating the subject with the IO therapy. In some aspects, the method further comprising treating the subject with an additional anti-cancer therapy. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

In some aspects, provided herein is a method where the chemotherapeutic agent comprises one or more of an alkylating agent, an alkyl sulfonates aziridine, an ethylenimine, a methylamelamine, an acetogenin, a camptothecin, a bryostatin, a callystatin, CC-1065, a cryptophycin, aa dolastatin, a duocarmycin, a eleutherobin, a pancratistatin, a sarcodictyin, a spongistatin, a nitrogen mustard, a nitrosureas, an antibiotic, a dynemicin, a bisphosphonate, an esperamicina a neocarzinostatin chromophore or a related chromoprotein enediyne antibiotic chromophore, an anti-metabolite, a folic acid analogue, a purine analog, a pyrimidine analog, an androgens, an anti-adrenal, a folic acid replenisher, aldophosphamide glycoside, aminolevulinic acid, eniluracil, amsacrine, bestrabucil, bisantrene, edatraxate, defofamine, demecolcine, diaziquone, elformithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, a PSK polysaccharide complex, razoxane, rhizoxin, sizofiran, spirogermanium, tenuazonic acid, triaziquone, 2,2′,2″-trichlorotriethylamine, a trichothecene, urethan, vindesine, dacarbazine, mannomustine, mitobronitol, mitolactol, pipobroman, gacytosine, arabinoside (“Ara-C”), cyclophosphamide, a taxoid, 6-thioguanine, mercaptopurine, a platinum coordination complex, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluorometlhylomithine (DMFO), a retinoid, capecitabine, carboplatin, procarbazine, plicomycin, gemcitabine, navelbine, farnesyl-protein transferase inhibitors, transplatinum, or any combination thereof.

In some aspects, provided herein is a method where the cancer is a B cell cancer, a melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer or carcinoma, lung non-small cell lung carcinoma (NSCLC), head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In any of the provided embodiments, the subject is a human. In any of the provided embodiments, the subject has previously been treated with an anti-cancer therapy. In some embodiments, the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

Also provided herein is a system comprising one or more processors, and a memory communicatively coupled to the one or more processors and configured to store instructions that, when executed by the one or more processors, cause the system to receive a plurality of sequence read data that represent a set of nucleic acid molecules obtained from a sample for a subject; identify one or more short variants from the sequence read data; filter the one or more short variants to select one or more clonal tumor mutational burden (cTMB)-qualified short variants, wherein the filtering comprises excluding short variants identified as subclonal based on a tumor amount measure for a respective short variant; and determine a cTMB value (for example, a cTMB score) for the sample based on the number of cTMB-qualified short variants.

Also provided herein is a system comprising one or more processors, and a memory communicatively coupled to the one or more processors and configured to store instructions that, when executed by the one or more processors, cause the system to receive genomic data for a subject, wherein the genomic data indicates a presence or absence of one or more short variants in a sample derived from the subject; filter the one or more short variants to select one or more clonal tumor mutational burden (cTMB)-qualified short variants, wherein the filtering comprises excluding short variants identified as subclonal based on a tumor amount measure for a respective short variant; and determine a cTMB value (for example, a cTMB score) for the sample based on the number of cTMB-qualified short variants.

In some aspects, the instructions further cause the system to select a treatment for the subject, wherein the subject has a cancer, based on the cTMB for the sample. In some embodiments, if the cTMB determined for the sample is at or above a threshold cTMB, the subject is identified as one who may benefit from treatment with an immune-oncology (IO) therapy.

In some aspects, the instructions further cause the system to generate a report comprising one or more treatment options identified for the subject based at least in part on the cTMB determined for the sample, wherein if the cTMB is at or above a threshold cTMB, the subject is identified as one who may benefit from treatment with an immune-oncology (IO) therapy.

In some aspects, the instructions further cause the system to predict survival of the subject, wherein the subject has cancer, wherein if the cTMB determined for the sample is at or above a threshold cTMB, the subject is predicted to have longer survival when treated with an immune-oncology (IO) therapy, as compared to a subject determined to have a cTMB below the threshold cTMB.

In some aspects, provided herein is a system where the cTMB is determined with one or more of (a) counting a number of the one or more clonal short variants in the sample, and (b) normalizing a size of a bait molecule. In some embodiments, the tumor amount measure comprises a cancer cell fraction (CCF) of the short variant or a variant allele frequency (VAF). In some embodiments, the filter step comprises excluding short variants identified as having an allele frequency below a predetermined threshold. In some embodiments, the filter step comprises excluding short variants identified as a germline variant. In some embodiments, the filter step comprises excluding short variants identified as having a cancer cell fraction (CCF) at or below a predetermined CCF threshold.

In some aspects, provided herein is a system where a clonal tumor mutational burden (cTMB) value (for example, a cTMB score) is expressed as a function of the number of cTMB-qualified short variants per megabase (mutations/Mb) in a set of subgenomic intervals from the sample. In some embodiments, the set of subgenomic intervals from the sample are between about 100 kb to about 10 Mb. In some embodiments, the set of subgenomic intervals from the sample are between about 0.8 Mb to about 1.1 Mb.

In some aspects, provided herein is a system where the genomic data for the subject is based on a targeted exome sequencing panel. In some embodiments, the genomic data for the subject is derived from a single biopsy sample. In some embodiments, the genomic data for the subject is derived from only one biopsy sample. In some embodiments, the genomic data for the subject is derived from circulating tumor DNA in a liquid biopsy sample. In some embodiments, the genomic data for the subject is derived from single cell sequencing. In some embodiments, the one or more short variants include noncoding and synonymous short variants.

In some aspects, provided herein is a system further comprising obtaining the sample from the subject. In some embodiments, the sample comprises a tissue biopsy sample, a liquid biopsy sample, or a normal control. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof.

In some aspects, provided herein is a system where the plurality of nucleic acid molecules comprises a mixture of tumor nucleic acid molecules and non-tumor nucleic acid molecules. In some embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In some embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In some aspects, provided herein is a system where the genomic data is obtained from sequencing the sample. In some embodiments, the sequencing comprises use of a massively parallel sequencing (MPS) technique, whole genome sequencing (WGS), whole exome sequencing, targeted sequencing, direct sequencing, or Sanger sequencing technique. In some embodiments, the sequencing comprises massively parallel sequencing, and the In some embodiments, the sequencing comprises providing a plurality of nucleic acid molecules obtained from the sample from the subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the set of nucleic acid molecules in the sample.

In some aspect, provided herein is a system where the one or more adapters comprise amplification primers, flow cell adapter sequences, unique molecular identifier sequence, substrate adapter sequences, or sample index sequences. In some embodiments, the captured nucleic acid molecules are captured from the amplified nucleic acid molecules by hybridization to one or more bait molecules. In some embodiments, the one or more bait molecules comprise one or more nucleic acid molecules, each comprising a region that is complementary to a region of a captured nucleic acid molecule. In some embodiments, amplifying nucleic acid molecules comprises performing a polymerase chain reaction (PCR) amplification technique, a non-PCR amplification technique, or an isothermal amplification technique.

In some aspects, the plurality of sequence reads overlap one or more gene loci within a subgenomic interval in the sample. In some embodiments, the one or more short variants comprise ALOX12B T402T, ATRX K2225del, CTNNA1 D362N, FLT3 S102S, JAK2 I289V, KEAP1 K97N, KRAS Q61H, MSH3 R573fs*4, PDK1 T306M, STAG2 C527F, STK11 649_650delCC, TSC2 A68S, or any combination thereof. In some embodiments, the one or more cTMB-qualified short variants comprise CTNNA1 D362N, FLT3 S102S, STAG2 C527F, TSC2 A68S, or any combination thereof.

In some aspects, provided herein in a system further comprising generating, by the one or more processors, a report comprising a cTMB for the subject. In some aspects, the system further comprises transmitting the report to a healthcare provider. In some embodiments, the report is transmitted via a computer network or a peer-to-peer connection.

In some aspects, a threshold cTMB value (for example, a cTMB score) is at least about 4 to 100 mutations/Mb, about 4 to 30 mutations/Mb, 8 to 100 mutations/Mb, 8 to 30 mutations/Mb, 10 to 20 mutations/Mb, less than 4 mutations/Mb, or less than 8 mutations/Mb. In some embodiments, a threshold cTMB value is at least about 5 mutations/Mb, at least about 10 mutations/Mb, at least about 12 mutations/Mb, at least about 16 mutations/Mb, at least about 20 mutations/Mb, or at least about 30 mutations/Mb. In some embodiments, a threshold cTMB value is at least about 7.5 mutations/Mb, at least about 8.5 mutations/Mb, at least about 9.5 mutations/Mb, at least about 11 mutations/Mb, at least about 13.5 mutations/Mb, at least about 16.5 mutations/Mb, at least about 18.5 mutations/Mb, at least about 19 mutations/Mb, or at least about 25 mutations/Mb.

In some aspects, the immuno-oncology (IO) therapy comprises a single IO agent or multiple IO agents. In some embodiments, the immuno-oncology (IO) therapy comprises an immune checkpoint inhibitor. In some embodiments, the immune checkpoint inhibitor comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a cellular therapy, a treatment for cancer being tested in a clinical trial, an immunotherapy, or any combination thereof.

In some embodiments, the immune checkpoint inhibitor is a PD-1 inhibitor. In some embodiments, the immune checkpoint inhibitor comprises one or more of nivolumab, pembrolizumab, cemiplimab, or dostarlimab. In some embodiments, the immune checkpoint inhibitor is a PD-L1-inhibitor. In some embodiments, the immune checkpoint inhibitor comprises one or more of atezolizumab, avelumab, or durvalumab. In some embodiments, the immune checkpoint inhibitor is a CTLA-4 inhibitor. In some embodiments, the CTLA-4 inhibitor comprises ipilimumab. In some embodiments, the nucleic acid comprises a double-stranded RNA (dsRNA), a small interfering RNA (siRNA), or a small hairpin RNA (shRNA). In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In aspects, provided herein is a system further comprising treating the subject with the IO therapy. In some aspects, the system further comprises treating the subject with an additional anti-cancer therapy.

In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

In some aspects, provided herein is a system where the chemotherapeutic agent comprises one or more of an alkylating agent, an alkyl sulfonates aziridine, an ethylenimine, a methylamelamine, an acetogenin, a camptothecin, a bryostatin, a callystatin, CC-1065, a cryptophycin, aa dolastatin, a duocarmycin, a eleutherobin, a pancratistatin, a sarcodictyin, a spongistatin, a nitrogen mustard, a nitrosureas, an antibiotic, a dynemicin, a bisphosphonate, an esperamicina a neocarzinostatin chromophore or a related chromoprotein enediyne antibiotic chromophore, an anti-metabolite, a folic acid analogue, a purine analog, a pyrimidine analog, an androgens, an anti-adrenal, a folic acid replenisher, aldophosphamide glycoside, aminolevulinic acid, eniluracil, amsacrine, bestrabucil, bisantrene, edatraxate, defofamine, demecolcine, diaziquone, elformithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, a PSK polysaccharide complex, razoxane, rhizoxin, sizofiran, spirogermanium, tenuazonic acid, triaziquone, 2,2′,2″-trichlorotriethylamine, a trichothecene, urethan, vindesine, dacarbazine, mannomustine, mitobronitol, mitolactol, pipobroman, gacytosine, arabinoside (“Ara-C”), cyclophosphamide, a taxoid, 6-thioguanine, mercaptopurine, a platinum coordination complex, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluorometlhylomithine (DMFO), a retinoid, capecitabine, carboplatin, procarbazine, plicomycin, gemcitabine, navelbine, farnesyl-protein transferase inhibitors, transplatinum, or any combination thereof.

In some aspects, provided herein is a system where the cancer is a B cell cancer, a melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer or carcinoma, lung non-small cell lung carcinoma (NSCLC), head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some aspects, the subject is a human. In some embodiments, the subject has previously been treated with an anti-cancer therapy. In some embodiments, the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a schematic diagram depicting the calculation of clonal tumor mutational burden (cTMB), in accordance with some embodiments.

FIG. 2 provides another schematic diagram depicting the criteria used in the calculation of clonal tumor mutational burden (cTMB), according to some embodiments.

FIG. 3A provides a first part of a study design/cohort diagram for a cohort of non-small cell lung cancer (NSCLC) patient samples from the clinic-genomic database (CGDB) used in evaluating clonal tumor mutational burden (cTMB) as a biomarker for the patient's response to immune checkpoint inhibitors (ICI) therapy, in accordance with some instances of the methods and systems described herein. FIG. 3B provides a second part of a study design/cohort diagram described for FIG. 3A. FIG. 3C provides a heatmap depicting the quality control parameters amongst the QC failed specimens.

FIG. 4 provides a second part of a study design/cohort diagram for a cohort of non-small cell lung cancer (NSCLC) patient samples from the clinic-genomic database (CGDB) used in evaluating clonal tumor mutational burden (cTMB) as a biomarker for the patient's response to immune checkpoint inhibitors (ICI) therapy, in accordance with some instances of the methods and systems described herein.

FIGS. 5A-5T provide Univariate Kaplan Meier and Cox Proportional Hazards models for a tumor mutational burden (TMB) and clonal TMB (cTMB) of the Line 2 cohort for various percentile thresholds, as shown in Table 1 and Table 2. FIG. 5A provides a Univariate Kaplan Meier plot showing overall survival (OS) probability versus time from start of second line immune checkpoint inhibitor (ICI) monotherapy treatment for the NSCLC patient cohort stratified by tumor mutational burden (TMB) value, where the TMB threshold is equal to median of the TMB of the Line 2 cohort, in accordance with some instances of the methods and systems described herein. FIG. 5B provides a Cox Proportional Hazards model showing the results for a Cox proportional hazards regression model from start of second line immune checkpoint inhibitor (ICI) monotherapy treatment for the NSCLC patient cohort stratified by tumor mutational burden (TMB) value, where the TMB threshold is equal to median of the TMB of the Line 2 cohort, in accordance with some instances of the methods and systems described herein. FIG. 5C provides a Univariate Kaplan Meier plot showing overall survival (OS) probability versus time from start of second line immune checkpoint inhibitor (ICI) monotherapy treatment for the NSCLC patient cohort stratified by clonal tumor mutational burden (cTMB) value, where cTMB threshold equal to median of the cTMB of the Line 2 cohort, in accordance with some instances of the methods and systems described herein. FIG. 5D provides a Cox Proportional Hazard models showing the results for a Cox proportional hazards regression model from start of second line immune checkpoint inhibitor (ICI) monotherapy treatment for the NSCLC patient cohort stratified by clonal tumor mutational burden (cTMB) value, where the cTMB threshold equal to median of the cTMB of the Line 2 cohort, in accordance with some instances of the methods and systems described herein. FIG. 5E provides a Univariate Kaplan Meier plot, similar to FIG. 5A, where the TMB threshold equal to 60^{th} percentile of TMB of the Line 2 cohort. FIG. 5F provides a Cox Proportional Hazards model, similar to FIG. 5B, where the TMB threshold equal to 60^{th} percentile of TMB of the Line 2 cohort. FIG. 5G provides a Univariate Kaplan Meier plot, similar to FIG. 5C, where the cTMB threshold equal to 60^{th} percentile of cTMB of the Line 2 cohort. FIG. 5H provides a Cox Proportional Hazards model, similar to FIG. 5D, where the cTMB threshold equal to 60^{th} percentile of cTMB of the Line 2 cohort. FIG. 5I provides a Univariate Kaplan Meier plot, similar to FIG. 5A, where the TMB threshold equal to 70^{th} percentile of TMB of the Line 2 cohort. FIG. 5J provides a Cox Proportional Hazards model, similar to FIG. 5B, where the TMB threshold equal to 70^{th} percentile of TMB of the Line 2 cohort. FIG. 5K provides a Univariate Kaplan Meier plot, similar to FIG. 5C, where the cTMB threshold equal to 70^{th} percentile of cTMB of the Line 2 cohort. FIG. 5L provides a Cox Proportional Hazards model, similar to FIG. 5D, where the cTMB threshold equal to 70^{th} percentile of cTMB of the Line 2 cohort. FIG. 5M provides a Univariate Kaplan Meier plot, similar to FIG. 5A, where the TMB threshold equal to 80^{th} percentile of TMB of the Line 2 cohort. FIG. 5N provides a Cox Proportional Hazards model, similar to FIG. 5B, where the TMB threshold equal to 80^{th} percentile of TMB of the Line 2 cohort. FIG. 5O provides a Univariate Kaplan Meier plot, similar to FIG. 5C, where the cTMB threshold equal to 80^{th} percentile of cTMB of the Line 2 cohort. FIG. 5P provides a Cox Proportional Hazards model, similar to FIG. 5D, where the cTMB threshold equal to 80^{th} percentile of cTMB of the Line 2 cohort. FIG. 5Q provides a Univariate Kaplan Meier plot, similar to FIG. 5A, where the TMB threshold equal to 90^{th} percentile of TMB of the Line 2 cohort. FIG. 5R provides a Cox Proportional Hazards model, similar to FIG. 5B, where the TMB threshold equal to 90^{th} percentile of TMB of the Line 2 cohort. FIG. 5S provides a Univariate Kaplan Meier plot, similar to FIG. 5C, where the cTMB threshold equal to 90^{th} percentile of cTMB of the Line 2 cohort. FIG. 5T provides a Cox Proportional Hazards model, similar to FIG. 5D, where the cTMB threshold equal to 90^{th} percentile of cTMB of the Line 2 cohort.

FIG. 6 depicts an exemplary computing device, in accordance with some instances of the systems describe herein.

FIG. 7 depicts an exemplary computer system or computer network, in accordance with some instances of the systems described herein.

### DETAILED DESCRIPTION

Described herein are methods for profiling a fraction of the genome or exome from a patient sample (e.g., short variants, using a hybrid capture-based, next-generation sequencing (NGS) platform) to determine clonal tumor mutational burden (cTMB), as well as methods for treating subjects or selecting a treatment for a subject based on the determined cTMB value. These short variants derived from the sequence read can be filtered to select for cTMB-qualified short variants, while excluding short variants identified as subclonal based on a cancer cell fraction (CCF) amount measure for a respective short variant. Importantly, the clonality of the short variants underlying the TMB value may impact the probability of the short variants/neoantigens being presented to the immune system. Subclonal neoantigens are less likely to be presented on the surface of the tumor by the human leukocyte antigen (HLA) mechanism as compared to clonal neoantigens. Clonal neoantigens can be recognized by the immune system (e.g., T cells) and may play an important role in checkpoint inhibitor response. Therefore, cTMB may be a more accurate TMB metric and clinically similar to or better than TMB for stratifying likelihood of cancer immunotherapy response. The cTMB determined according to the methods described herein provides a new metric useful for determining mutational load of a tumor sample and the likelihood of immunotherapy response to immune checkpoint inhibitors.

The likelihood of generating immunogenic tumor neoantigens is understood to increase in a probabilistic fashion as mutations develop, increasing the likelihood of immune recognition (Gubin et al., CANCER. The odds of immunotherapy success, Science, vol. 350, no. 6257, pp. 158-159 (2015)). A high tumor mutational burden (TMB-H) is further associated with the presence of activated effector T cells that results in enhanced responsiveness to immune checkpoint inhibition, e.g., Snyder et al., Genetic basis for clinical response to CTLA-4 blockade in melanoma, NEJM, vol. 37, no. 23, pp. 2189-2199 (2014) and McGranahan et al., Tumor Heterogeneity Correlates with Less Immune Response and Worse Survival in Breast Cancer Patients, Science, vol. 351, no. 6280, pp. 1463-1469 (2016). It follows that TMB-H (at least 10 mutations per megabase) is an accepted biomarker of immune checkpoint inhibitor (ICI) response for metastatic solid tumors. However, the clonality of the neoantigens (e.g., clonal or subclonal neoantigens) may impact the quality and clinical value of the TMB value. Subclonal neoantigens are less likely to be presented on the surface of the tumor by the human leukocyte antigen (HLA) mechanism as compared to clonal neoantigens. Clonal neoantigens can be recognized by the immune system more readily and play an important role in ICI response. Therefore, clonal TMB (cTMB) may represent a more accurate TMB metric for understanding mutational load in a tumor sample. Further, the cTMB may be clinically similar to or better than TMB for stratifying patient likelihood of cancer immunotherapy response. Provided herein are methods for determining cTMB and various uses thereof, including but not limited to clinical applications.

The methods provided herein have several pragmatic advantages, including, for example, more clinically-feasible turnaround times, standardized informatics pipelines, and more manageable costs. Compared to multi-region biopsies, which are difficult to execute in a routine clinical care setting, the provided methods using targeted exome sequencing can be performed routinely in the clinic. Further, the evaluation of clonality is simplified, leveraging a cancer cell fraction (CCF) estimation as opposed to using mixture modelling techniques, which are prone have demonstrated limited predictive power. Collectively, the provided methods related to determining cTMB and uses thereof are superior to previous TMB metrics and more convenient that other approaches of filtering for clonality that rely on whole genome sequencing and multi-region biopsies. This approach also has other advantages over traditional markers, such as protein expression detected by histochemistry, since it produces an objective (e.g., mutation load) rather than a subjective measure (pathology scoring) (Hansen et al., PD-L1 Testing in Cancer: Challenges in Companion Diagnostic Development, JAMA Oncol., vol. 2, no. 1, pp. 15-16 (2016)). Further, this platform facilitates simultaneous detection of actionable alterations relevant for targeted therapies.

Accordingly, the invention provides, at least in part, methods comprising obtaining, using one or more processors, a plurality of sequence read data that represent a set of nucleic acid molecules obtained from a sample for a subject; identifying, using the one or more processors, one or more short variants from the sequence read data; filtering, using the one or more processors, the one or more short variants to select one or more clonal tumor mutational burden (cTMB)-qualified short variants, wherein the filtering comprises excluding short variants identified as subclonal based on a tumor amount measure for a respective short variant; and determining, using the one or more processors, a cTMB value for the sample based on the number of cTMB-qualified short variants. For example, the tumor amount measure for a respective short variant may comprise a cancer cell fraction (CCF) of the variant or a variant allele frequency (VAF). In other aspects, the invention provides, at least in part, methods comprising receiving, at one or more processors, genomic data for a subject, wherein the genomic data indicates a presence or absence of one or more short variants in a sample derived from the subject; filtering, using the one or more processors, the one or more short variants to select one or more clonal tumor mutational burden (cTMB)-qualified short variants, wherein the filtering comprises excluding short variants identified as subclonal based on a tumor amount measure for a respective short variant; and determining, using the one or more processors, a cTMB value for the sample based on the number of cTMB-qualified short variants.

In some embodiments, the clonal tumor mutational burden (cTMB) is determined by one or more of (a) counting a number of the one or more clonal short variants in the sample, and (b) normalizing the count of the clonal variants by the length of the genome sequenced by the assay (e.g., a baitset size). In some embodiments, the tumor amount measure comprises a cancer cell fraction (CCF) or a variant allele frequency (VAF). In some embodiments, the filtering comprises excluding short variants identified as having an allele frequency below a predetermined threshold, excluding short variants identified as a germline variant, and/or excluding short variants identified as having a CCF at or below a predetermined CCF threshold. In some embodiments, a clonal tumor mutational burden (cTMB) value is expressed as a function of the number of cTMB-qualified short variants per megabase (mutations/Mb) in a set of subgenomic intervals from the sample (e.g., between about 0.8 Mb to about 1.1 Mb).

Also provided herein are methods of treating a subject having a cancer, methods of selecting a treatment for a subject having a cancer, methods of identifying a subject having a cancer for treatment with an immune-oncology (IO) therapy, methods of identifying one or more treatment options for a subject having a cancer, and methods of predicting survival of a subject having cancer, each of which comprises determining a clonal tumor mutational burden (cTMB) value. Systems for evaluating the cTMB in a sample are also disclosed.

The disclosed methods and systems eliminate the need for methods requiring whole genome sequencing and multi-region biopsies for determining mutation load of a tumor sample. The disclosed methods and systems for determining clonal tumor mutational burden (cTMB) provide a superior biomarker and/or metric for evaluating mutation load in a tumor sample and for subsequent clinical applications and management of a patient (e.g., a patient having cancer).

### I. DEFINITIONS

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

The section headings used herein are for organizational purposes only and are not to be construed as limiting the subject matter described.

### II. METHODS FOR DETERMINING CLONAL TUMOR MUTATIONAL BURDEN (CTMB)

In some aspects, provided herein is a method comprising obtaining, using one or more processors, a plurality of sequence read data that represent a set of nucleic acid molecules obtained from a sample for a subject. In some embodiments, the method further comprises identifying, using the one or more processors, one or more short variants from the sequence read data. In some embodiments, the method further comprises filtering, using the one or more processors, the one or more short variants to select one or more clonal tumor mutational burden (cTMB)-qualified short variants. In some embodiments, the filtering comprises excluding short variants identified as subclonal based on a tumor amount measure for a respective short variant. For example, the tumor amount measure for a respective short variant may comprise a cancer cell fraction (CCF) of the variant or a variant allele frequency (VAF). In some embodiments, the method further comprises determining, using the one or more processors, a cTMB value (for example, a cTMB score) for the sample based on the number of cTMB-qualified short variants.

In some aspects, provided herein is a method comprising obtaining, using one or more processors, a plurality of sequence read data that represent a set of nucleic acid molecules obtained from a sample for a subject; identifying, using the one or more processors, one or more short variants from the sequence read data; filtering, using the one or more processors, the one or more short variants to select one or more clonal tumor mutational burden (cTMB)-qualified short variants, where the filtering comprises excluding short variants identified as subclonal based on a tumor amount measure for a respective short variant; and determining, using the one or more processors, a cTMB value (for example, a cTMB score) for the sample based on the number of cTMB-qualified short variants. For example, the tumor amount measure for a respective short variant may comprise a cancer cell fraction (CCF) of the variant or a variant allele frequency (VAF).

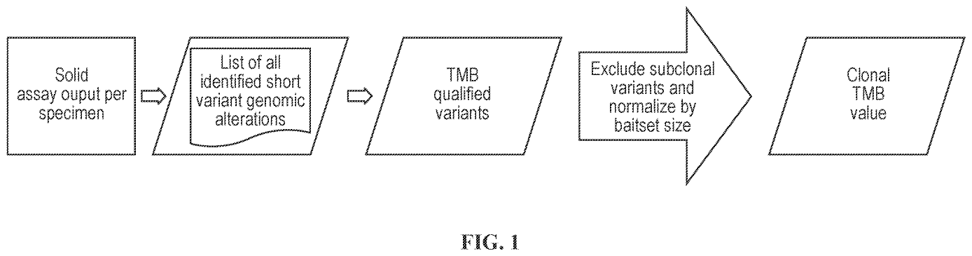

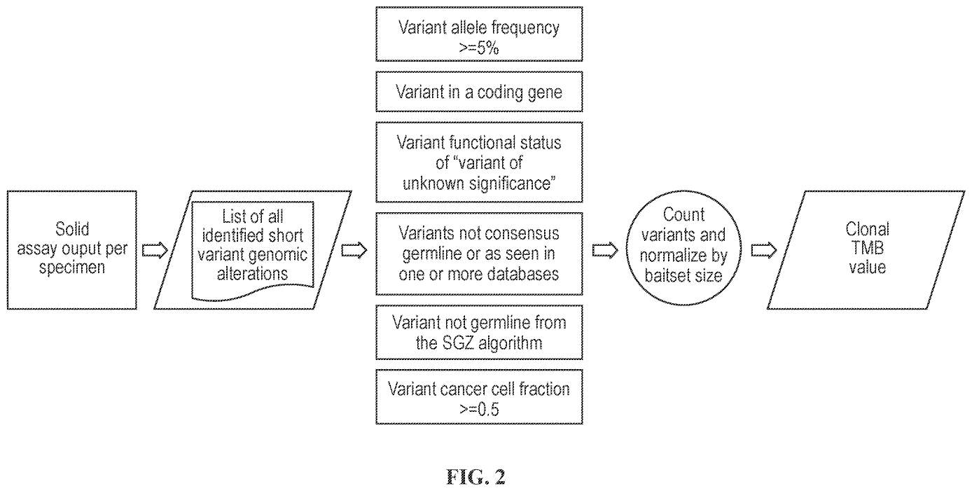

For example, as shown in FIG. 1, sequence read data, for example as obtained by sequencing nucleic acid molecules obtained from a sample from a subject, can be analyzed to identify one or more short variants to generate a list of identified short variant genomic alterations. The list of short variants may be filtered to identify cTMB-qualified short variants, as further described herein. The number of cTMB-qualified short variants may be normalized, for example, depending on the number of capture probes (baits) used during targeted sequencing or the length of the portion (e.g., coding region) of the genome sequenced. By normalizing the number of cTMB-qualified short variants, wider sequencing assays (i.e., those sequencing a larger number of genomic loci) provide a cTMB comparable to narrower sequencing assays (i.e., those sequencing a smaller number of genomic loci).

In some aspects, provided herein is a method comprising receiving, at one or more processors, genomic data for a subject, wherein the genomic data indicates a presence or absence of one or more short variants in a sample derived from the subject. In some embodiments, the method further comprises filtering, using the one or more processors, the one or more short variants to select one or more clonal tumor mutational burden (cTMB)-qualified short variants, wherein the filtering comprises excluding short variants identified as subclonal based on a tumor amount measure for a respective short variant. For example, the tumor amount measure for a respective short variant may comprise a cancer cell fraction (CCF) of the variant or a variant allele frequency (VAF). In some embodiments, the method further comprises determining, using the one or more processors, a cTMB value (for example, a cTMB score) for the sample based on the number of cTMB-qualified short variants.

In some embodiments, the method comprising the filtering described herein comprises one or more filtering criteria for filtering the one or more short variants to select one or more clonal tumor mutational burden (cTMB)-qualified short variants. In some embodiments, the filtering criteria comprise at least one, at least two, at least three, at least four, at least five, at least six, at least seven, at least eight, at least nine, at least 10 or more criteria for filtering the one or more short variants to select one or more cTMB-qualified short variants. In some embodiments, the filtering criteria comprises a relation to one or more somatic variants. In some embodiments, the filtering criteria comprises a relation to one or more germline variants. In some embodiments, the filtering criteria comprises a relation to a coding region. In some embodiments, the filtering criteria comprises a relation to a non-coding region and/or a coding gene. In some embodiments, the filtering criteria comprises a relation to a coding gene and/or a non-code. In some embodiments, the filtering criteria comprises a relation to the functional status of the short variant. In some embodiments, the filtering criteria comprises a relation to a short variant of unknown significance. In some embodiments, the filtering criteria comprises a relation to a short variant that meets a certain variant allele frequency (VAF) threshold. In some embodiments, the filtering criteria comprises a relation to a short variant that meets a certain cancer cell fraction (CCF) threshold.

In some embodiments, the method comprising the filtering criteria provided herein comprises at least one criteria selected from the group consisting of a variant allele frequency (VAF) threshold, a variant in a coding gene, a variant of unknown significance, a variant determined not to be a germline variant, and a variant cancer cell fraction (CCF) threshold. In some embodiments, the filtering criteria provided herein comprises at least two criteria selected from the group consisting of a VAF threshold, a variant in a coding gene, a variant of unknown significance, a variant determined not to be a germline variant, and a variant CCF threshold. In some embodiments, the filtering criteria provided herein comprises at least three criteria selected from the group consisting of a VAF threshold, a variant in a coding gene, a variant of unknown significance, a variant determined not to be a germline variant, and a variant CCF threshold. In some embodiments, the filtering criteria provided herein comprises at least four criteria selected from the group consisting of a VAF threshold, a variant in a coding gene, a variant of unknown significance, a variant determined not to be a germline variant, and a variant CCF threshold. In some embodiments, the filtering criteria provided herein comprises each criteria selected from the group consisting of a VAF threshold, a variant in a coding gene, a variant of unknown significance, a variant determined not to be a germline variant, and a variant CCF threshold.

In some aspects, provided herein is a method comprising obtaining, using one or more processors, a plurality of sequence read data that represent a set of nucleic acid molecules obtained from a sample for a subject; identifying, using the one or more processors, one or more short variants from the sequence read data; filtering, using the one or more processors, the one or more short variants to select one or more clonal tumor mutational burden (cTMB)-qualified short variants, where the filtering comprises excluding short variants identified as subclonal based on a tumor amount measure for a respective short variant, where the filtering criteria comprises a VAF threshold, a variant in a coding gene, a variant of unknown significance, a variant determined not to be a germline variant, and a variant CCF threshold; and determining, using the one or more processors, a cTMB value (for example, a cTMB score) for the sample based on the number of cTMB-qualified short variants.

For example, as shown in FIG. 2, sequence read data, for example as obtained by sequencing nucleic acid molecules obtained from a sample from a subject, can be analyzed to identify one or more short variants to generate a list of identified short variant genomic alterations. The list of short variants may be filtered to identify cTMB-qualified short variants, as further described herein. The list of short variants may be filtered, for example, to exclude one or more of (1) short variants having a variant allele frequency below a predetermined threshold, (2) short variants in a non-coding region of the genome, (3) short variants identified as being a germline variant, (4) short variants with a known or likely functional status, other than a variant of unknown significance, and/or (5) short variants with a variant cancer cell fraction (CCF) less than a predetermined threshold. The number of cTMB-qualified short variants may be normalized (e.g., by the length of the coding genome sequenced in megabases), for example depending on the number of capture probes (baits) used during targeted sequencing.

In some embodiments, the method comprising the filtering criteria comprises one or more filtering criteria related to a variant allele frequency (VAF) threshold. In some embodiment, the one or more short variants is included for analysis if it meets a certain VAF threshold. In some embodiment, the one or more short variants is not included for analysis if it does not meet a certain VAF threshold. In some embodiments, the VAF threshold for filtering the one or more short variants is at least 0.1%, at least 0.5%, at least 1%, at least 2%, at least 3%, at least 4%, at least 5%, at least 10% or more. In some embodiments, the VAF threshold for filtering the one or more short variants is between 0.1% and 30%, 0.5% and 25%, 1% and 20%, 2% and 15%, 3% and 10%, 4% and 9%, 5% and 9%, or 6% and 8%. In some embodiments, the VAF threshold for filtering the one or more short variants is at least 0.1%. In some embodiments, the VAF threshold for filtering the one or more short variants is at least 1%. In some embodiments, the VAF threshold for filtering the one or more short variants is at least 5%. In some embodiments, the VAF threshold for filtering the one or more short variants is at least 10%.

In some embodiments, the method comprising the filtering criteria comprise one or more filtering criteria related to a variant in a coding gene. In some embodiment, the one or more short variants is included for analysis if the variant is located in a coding region and/or coding gene. In some embodiment, the one or more short variants is not included for analysis if the variant is located in a non-coding region, for example, an intron, a regulatory element, and/or an intergenic region.

In some embodiments, the method comprising the filtering criteria comprise one or more filtering criteria related to the significance of a variant. In some embodiment, the one or more short variants is included for analysis if the variant is a variant of unknown significance (VUS), e.g., an alteration of which the pathogenicity of which can neither be confirmed nor ruled out. In some embodiments, the VUS is an alteration (e.g., somatic alteration) which has not been identified as being associated with a cancer phenotype. In some embodiments, the one or more short variants is included for analysis if the variant is a variant of uncertain significance. In some embodiment, the one or more short variants is not included for analysis if the variant is a variant of known significance. For example, a variant of known significance may comprise an alteration that is oncogenic, impacts protein functionality, expression level, signaling, another biological aspect, or combination thereof. In some embodiment, the one or more short variants is not included for analysis if the variant is a non-functional variant. For example, a variant that is known to exist but does not impact fitness of a clone of a cell (e.g., the alteration is a silent mutation, such as a synonymous alteration).

In some embodiments, the method comprising the filtering criteria comprises one or more filtering criteria for excluding a germline variant. In some embodiments, the filtering criteria for excluding a germline variant involves comparing one or more short variants to a curated database. The curated database is one or more genomic databases that associates a genomic profile from an individual having a disease with a disease type, for example, a cancer type (e.g., metastatic lung cancer). In some embodiments, comparing one or more short variants to a curated genomic database confirms the presence of a consensus germline variant. In some embodiments, the filtering criteria for excluding a germline variant involves analyzing one or more short variants with an algorithm that selects for somatic variants. In some embodiments, the filtering criteria for excluding a germline variant involves analyzing one or more short variants with an algorithm that identifies germline variants. In some embodiments, the algorithm is the SGZ algorithm. In some embodiments, a germline variant or mutation is identified in the one or more short variants by a method using the SGZ algorithm.

Various types of alterations, e.g., somatic alterations and germline mutations, can be detected by a method (e.g., a sequencing, alignment, or mutation calling method) described herein. In certain embodiments, a germline mutation is further identified by a method using the SGZ algorithm. The SGZ algorithm is described in Sun et al., A computational approach to distinguish somatic vs. germline origin of genomic alterations from deep sequencing of cancer specimens without a matched normali, PLOS Computational Biology, vol. 14, no. 2, pp. e1005965 (2018); International Application Publication No. WO2014/183078 and U.S. Application Publication No. 2014/0336996, the contents of which are incorporated by reference in their entirety. In certain embodiments, the germline mutation identified by a method using the SGZ algorithm is excluded from subsequent analyses described herein.

In some embodiments, the method comprising the filtering criteria comprise one or more filtering criteria related to a cancer cell fraction (CCF) threshold. In some embodiments, the CCF is the proportion of cancer cells in tumor sample containing the one or more short variants. In some embodiment, the one or more short variants is included for analysis if it meets a certain CCF threshold. In some embodiment, the one or more short variants is not included for analysis if it does not meet a certain CCF threshold. In some embodiments, the CCF is a value between 0 and 1, wherein the CCF value represents a percentage of cancer cells containing the one or more variant in the cancer cell population. For example, a CCF equal to 0.50 represents 50% of the cancer cell population contains the one or more short variant. In some embodiments, the CCF threshold for filtering the one or more short variants is at least 0.30, at least 0.35, at least 0.40, at least 0.45, at least 0.50, at least 0.55, at least 0.60, at least 0.65, at least 0.70, at least 0.75, at least 0.80, at least 0.85, at least 0.90, at least 0.95, at least 0.96, at least 0.97, at least 0.98, at least 0.99, at least 0.995, or more. In some embodiments, the CCF threshold for filtering the one or more short variants is at least 0.30 and less than 1.00. In some embodiments, the CCF threshold for filtering the one or more short variants is between 0.30 and 0.995, 0.35 and 0.95, 0.40 and 0.90, 0.45 and 0.85, 0.50 and 0.80, 0.55 and 0.75, or 0.60 and 0.70. In some embodiments, the CCF threshold for filtering the one or more short variants is at least 0.40. In some embodiments, the CCF threshold for filtering the one or more short variants is at least 0.45. In some embodiments, the CCF threshold for filtering the one or more short variants is at least 0.50. In some embodiments, the CCF threshold for filtering the one or more short variants is at least 0.55.

In some embodiments, the method comprising filtering the obtained sequence read data, using the one or more processors, to select one or more clonal tumor mutational burden (cTMB)-qualified short variants. In some embodiments, the method comprising filtering the received data, using the one or more processors, to select one or more cTMB-qualified short variants. In some embodiments, the filtering comprises excluding short variants identified as subclonal based on a tumor amount measure for a respective short variant. In some embodiments, the subclonal variant is a variant having a variant allele frequency (VAF) below a certain threshold. In some embodiments, the VAF threshold for identifying a subclonal variant is less than 10%, less than 9%, less than 7%, less than 6%, less than 5%, less than 4%, less than 3%, less than 2%, less than 1%, less than 0.5%, or less than 0.1%. In some embodiments, the VAF threshold for identifying a subclonal variant is less than 10%. In some embodiments, the VAF threshold for identifying a subclonal variant is less than 5%.

In some embodiments, the method further comprises determining, using the one or more processors, a clonal tumor mutational burden (cTMB) for the sample based on the number of cTMB-qualified short variants. In some embodiments, the number of cTMB-qualified short variants includes one or more variants that have been filtered based on one or more criteria including but not limited to a variant allele frequency (VAF) threshold, a variant in a coding gene, a variant of unknown significance, a variant determined not to be a germline variant, and a variant cancer cell fraction (CCF) threshold. In some embodiments, the number of cTMB-qualified short variants include one or more variants that meet the one or more criteria including but not limited to a VAF threshold, a variant in a coding gene, a variant of unknown significance, a variant determined not to be a germline variant, and a variant CCF threshold. In some embodiments, the number of cTMB-qualified short variants does not include one or more variants that do not meet the one or more criteria including but not limited to a VAF threshold, a variant in a coding gene, a variant of unknown significance, a variant determined not to be a germline variant, and a variant CCF threshold. In some embodiments, the number of cTMB-qualified short variants does not include one or more variants that have been identified as a subclonal variant.

In some embodiments, the method provided herein comprises obtaining, using one or more processors, a plurality of sequence read data from a sample for a subject. In some embodiments, the subject has a cancer. In some embodiments, the sample is a cancer sample. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some embodiments, the method comprises determining a clonal tumor mutational burden (cTMB). In some embodiments, the clonal tumor mutational burden (cTMB) is determined by counting a number of the one or more clonal short variants in the sample. In some embodiments, the cTMB is determined by normalizing a size of a bait molecule. In some embodiments, the cTMB is determined by normalizing by the length of the coding genome that is sequenced. In some embodiments, the cTMB is determined by normalizing a size of a bait molecule or by the length of a sequenced portion of the genome (e.g., the coding genome that is sequenced). In some embodiments, the cTMB is determined by counting a number of the one or more clonal short variants in the sample and by normalizing a size of a bait molecule.

In some embodiments, the method comprises the tumor amount measure comprising a cancer cell fraction (CCF) or a variant allele frequency (VAF). In some embodiments, the tumor amount measure comprises a CCF. In some embodiments, the CCF is a value between 0 and 1, wherein the CCF value represents a percentage of cancer cells containing the one or more variant in the cancer cell population. The CCF represents a simple but effective characteristic of a tumor sample. For example, a CCF equal to 0.50 represents 50% of the cancer cell population contains the one or more short variant. In some embodiments, the tumor amount measure comprises a CCF that is at least 0.30, at least 0.35, at least 0.40, at least 0.45, at least 0.50, at least 0.55, at least 0.60, at least 0.65, at least 0.70, at least 0.75, at least 0.80, or more. In some embodiments, the tumor amount measure comprises a CCF that is between 0.30 and 0.80, 0.35 and 0.75, 0.40 and 0.60, or 0.45 and 0.55. In some embodiments, the tumor amount measure comprises a CCF that is at least 0.40. In some embodiments, the tumor amount measure comprises a CCF that is at least 0.45. In some embodiments, the tumor amount measure comprises a CCF that is at least 0.50. In some embodiments, the tumor amount measure comprises a CCF that is at least 0.55. In some embodiments, the method comprises using the tumor amount measure having a CCF to determine a clonal tumor mutational burden (cTMB) for the sample associated with the tumor amount measure.

In some embodiments, the method comprises the tumor amount measure comprising a cancer cell fraction (CCF) or a variant allele frequency (VAF). In some embodiments, the tumor amount measure comprises a VAF. In some embodiments, the VAF is less than 10%, less than 9%, less than 7%, less than 6%, less than 5%, less than 4%, less than 3%, less than 2%, less than 1%, less than 0.5%, or less than 0.1%. In some embodiments, the VAF is no more than 10%, no more than 9%, no more than 7%, no more than 6%, no more than 5%, no more than 4%, no more than 3%, no more than 2%, no more than 1%, no more than 0.5%, or no more than 0.1%. In some embodiments, the VAF is less than 10%. In some embodiments, the VAF is no more than 10%. In some embodiments, the VAF is less than 5%. In some embodiments, the VAF is no more than 5%. In some embodiments, the method comprises using the tumor amount measure having a VAF to determine a clonal tumor mutational burden (cTMB) for the sample associated with the tumor amount measure.

In some embodiments, the method comprises filtering, using one or more processors, where the filtering comprises excluding short variants identified as having an allele frequency below a predetermined threshold. In some embodiments, the predetermined threshold for the variant allele frequency (VAF) is less than 10%, less than 9%, less than 7%, less than 6%, less than 5%, less than 4%, less than 3%, less than 2%, less than 1%, less than 0.5%, or less than 0.1%. In some embodiments, the predetermined threshold for the VAF is no more than 10%, no more than 9%, no more than 7%, no more than 6%, no more than 5%, no more than 4%, no more than 3%, no more than 2%, no more than 1%, no more than 0.5%, or no more than 0.1%. In some embodiments, the predetermined threshold for the VAF is less than 10%. In some embodiments, the predetermined threshold for the VAF is no more than 10%. In some embodiments, the predetermined threshold for the VAF is less than 5%. In some embodiments, the predetermined threshold for the VAF is no more than 5%. In some embodiments, the predetermined threshold for the VAF is used to determine if a variant is subclonal. In some embodiments, the filtering comprises excluding short variants identified as subclonal based on a tumor amount measure for a respective short variant. In some embodiments, the subclonal variant is a variant having a VAF below a certain threshold. In some embodiments, the method comprises using the tumor amount measure having a predetermined threshold for VAF to determine a clonal tumor mutational burden (cTMB) for the sample associated with the tumor amount measure.

In some embodiments, the method comprises filtering, using one or more processors, where the filtering comprises excluding short variants identified as a germline variant. In some embodiments, the method comprises using the tumor amount measure excluding germline variants to determine a clonal tumor mutational burden (cTMB) for the sample associated with the tumor amount measure.

In some embodiments, the filtering comprises excluding short variants identified as having a cancer cell fraction (CCF) at or below a predetermined CCF threshold.

In some embodiment, one or more short variants are not included for analysis if the one or more short variants are below a predetermined cancer cell fraction (CCF) threshold. In some embodiment, one or more short variants are included for analysis if the one or more short variants are at or above a predetermined CCF threshold. In some embodiments, the CCF is a value between 0 and 1, wherein the CCF value represents a percentage of cancer cells containing the one or more variant in the cancer cell population. For example, a CCF equal to 0.50 represents 50% of the cancer cell population contains the one or more short variant. In some embodiments, the predetermined CCF threshold for filtering the one or more short variants is at least 0.30, at least 0.35, at least 0.40, at least 0.45, at least 0.50, at least 0.55, at least 0.60, at least 0.65, at least 0.70, at least 0.75, at least 0.80, or more. In some embodiments, the predetermined CCF threshold for filtering the one or more short variants is between 0.30 and 0.80, 0.35 and 0.75, 0.40 and 0.60, or 0.45 and 0.55. In some embodiments, the predetermined CCF threshold for filtering the one or more short variants is at least 0.40. In some embodiments, the predetermined CCF threshold for filtering the one or more short variants is at least 0.45. In some embodiments, the predetermined CCF threshold for filtering the one or more short variants is at least 0.50. In some embodiments, the predetermined CCF threshold for filtering the one or more short variants is at least 0.55. In some embodiments, the method comprises using the tumor amount measure having a predetermined CCF threshold to determine a clonal tumor mutational burden (cTMB) for the sample associated with the tumor amount measure.

In some embodiments, a clonal tumor mutational burden (cTMB) value is expressed as a function of the number of cTMB-qualified short variants per megabase (mutations/Mb) in a set of subgenomic intervals from the sample. In some embodiments, the set of subgenomic intervals from the sample are between about 100 kb to about 10 Mb. In some embodiments, the set of subgenomic intervals from the sample are between about 100 kb to about 10 Mb, 200 kb to about 9 Mb, 300 kb to about 8 Mb, 400 kb to about 7 Mb, 500 kb to about 6 Mb, 600 kb to about 5 Mb, 700 kb to about 4 Mb, 800 kb to about 3 Mb, or 900 kb to about 2 Mb. In some embodiments, the set of subgenomic intervals from the sample are at least 0.1 Mb, 0.2 Mb, 0.3 Mb, 0.4 Mb, 0.5 Mb, 0.6 Mb, 0.7 Mb, 0.8 Mb, 0.9 Mb, 1 Mb, 2 Mb, 3 Mb, 4 Mb, 5 Mb, 6 Mb, 7 Mb, 8 Mb, 9 Mb, or more. In some embodiments, the set of subgenomic intervals from the sample are no more than 0.2 Mb, 0.3 Mb, 0.4 Mb, 0.5 Mb, 0.6 Mb, 0.7 Mb, 0.8 Mb, 0.9 Mb, 1 Mb, 2 Mb, 3 Mb, 4 Mb, 5 Mb, 6 Mb, 7 Mb, 8 Mb, 9 Mb, or 10 Mb. In some embodiments, the set of subgenomic intervals from the sample are between about 0.8 Mb to about 1.1 Mb. In some embodiments, the set of subgenomic intervals from the sample are between 0.8 Mb to 1.1 Mb.

In some embodiments, a clonal tumor mutational burden (cTMB) value is expressed as a function of the number of cTMB-qualified short variants per megabase (mutations/Mb) in a set of subgenomic intervals from the sample. In some embodiments, the cTMB value (for example, a cTMB score) from the sample is between about 0.1 mutations/Mb to about 100 mutations/Mb. In some embodiments, the cTMB value from the sample is between about 0.1 mutations/Mb to about 100 mutations/Mb, about 0.2 mutations/Mb to about 90 mutations/Mb, about 0.3 mutations/Mb to about 80 mutations/Mb, about 0.4 mutations/Mb to about 70 mutations/Mb, about 0.5 mutations/Mb to about 60 mutations/Mb, about 1 mutations/Mb to about 50 mutations/Mb, about 2 mutations/Mb to about 40 mutations/Mb, about 5 mutations/Mb to about 30 mutations/Mb, or about 10 mutations/Mb to about 20 mutations/Mb. In some embodiments, the cTMB value from the sample is at least 0.1 mutations/Mb, 0.2 mutations/Mb, 0.3 mutations/Mb, 0.4 mutations/Mb, 0.5 mutations/Mb, 1 mutations/Mb, 2 mutations/Mb, 5 mutations/Mb, 10 mutations/Mb, 15 mutations/Mb, 20 mutations/Mb, 30 mutations/Mb, 40 mutations/Mb, 50 mutations/Mb, 60 mutations/Mb, 70 mutations/Mb, 80 mutations/Mb, 90 mutations/Mb, or more. In some embodiments, the cTMB value from the sample is no more than 0.2 mutations/Mb, 0.3 mutations/Mb, 0.4 mutations/Mb, 0.5 mutations/Mb, 1 mutations/Mb, 2 mutations/Mb, 5 mutations/Mb, 10 mutations/Mb, 15 mutations/Mb, 20 mutations/Mb, 30 mutations/Mb, 40 mutations/Mb, 50 mutations/Mb, 60 mutations/Mb, 70 mutations/Mb, 80 mutations/Mb, 90 mutations/Mb, or 100 mutations/Mb. In some embodiments, the cTMB value from the sample is between about 5 mutations/Mb to about 20 mutations/Mb. In some embodiments, the cTMB value from the sample is between 5 mutations/Mb to 20 mutations/Mb.

In some embodiments, the genomic data for the subject is based on a targeted exome sequencing panel. In some embodiments, the genomic data for the subject is derived from a single sample. Examples of a sample include, but are not limited to, a tumor sample, a tissue sample, a biopsy sample, a blood sample, a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample. In some embodiments, the genomic data for the subject is derived from a single biopsy sample. In some embodiments, the genomic data for the subject is derived from only one biopsy sample. In some embodiments, the genomic data for the subject is derived from circulating tumor DNA in a liquid biopsy sample. In some instances, the liquid biopsy sample may comprise, for example, whole blood, blood plasma, blood serum, urine, stool, sputum, saliva, or cerebrospinal fluid. In some embodiments, the liquid biopsy sample may comprise circulating tumor cells (CTCs). In some embodiments, the liquid biopsy sample and may comprise cell-free DNA (cfDNA), circulating tumor DNA (ctDNA), or any combination thereof.

In some embodiments, the genomic data for the subject is derived from single cell sequencing. In some embodiments, the one or more short variants include noncoding and synonymous short variants.

In some embodiments, the method further comprises obtaining the sample from the subject. In some embodiments, the subject is a human. In some embodiments, the subject has a disease, for example, cancer. In some embodiments, the subject has previously been treated with an anti-cancer therapy. In some embodiments, the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof. In some embodiments, the subject has not previously been treated with an anti-cancer therapy. In some embodiments, the sample comprises a tissue biopsy sample, a liquid biopsy sample, or a normal control. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof.

In some embodiments, the set of nucleic acid molecules is a plurality of nucleic acid molecules. In some embodiments, the plurality of nucleic acid molecules comprises a mixture of tumor nucleic acid molecules and non-tumor nucleic acid molecules. In some embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In some embodiments, the sample comprises a liquid biopsy sample, where the tumor nucleic acid molecules are derived from a circulating tumor DNA (ctDNA) fraction of the liquid biopsy sample. In some embodiments, the sample comprises a liquid biopsy sample, and the non-tumor nucleic acid molecules are derived from a non-tumor, cell-free DNA (cfDNA) fraction of the liquid biopsy sample.

In some embodiments, the genomic data is obtained from sequencing the sample. In some embodiments, the sequencing comprises use of a massively parallel sequencing (MPS) technique, whole genome sequencing (WGS), whole exome sequencing, targeted sequencing, direct sequencing, Sanger sequencing technique, or a combination thereof. In some embodiments, the sequencing comprises massively parallel sequencing (MPS). In some embodiments, the massively parallel sequencing technique comprises next generation sequencing (NGS).

In some embodiments, the method comprises sequencing, where sequencing comprises providing a plurality of nucleic acid molecules obtained from the sample from the subject. In some embodiments, the sequencing further comprises ligating one or more adapters onto one or more nucleic acid molecules from the plurality of nucleic acid molecules. In some embodiments, the sequencing further comprises amplifying the one or more ligated nucleic acid molecules from the plurality of nucleic acid molecules. In some embodiments, the sequencing further comprises capturing amplified nucleic acid molecules from the amplified nucleic acid molecules. In some embodiments, the sequencing further comprises sequencing, by a sequencer, the captured nucleic acid molecules to obtain a plurality of sequence reads that represent the set of nucleic acid molecules in the sample.

In some embodiments, the one or more adapters comprise amplification primers, flow cell adapter sequences, unique molecular identifier sequence, substrate adapter sequences, sample index sequences, or a combination thereof. In some embodiments, the captured nucleic acid molecules are captured from the amplified nucleic acid molecules by hybridization to one or more bait molecules. In some embodiments, the one or more bait molecules comprise one or more nucleic acid molecules, each comprising a region that is complementary to a region of a captured nucleic acid molecule. In some embodiments, the amplifying nucleic acid molecules comprises performing a polymerase chain reaction (PCR) amplification technique, a non-PCR amplification technique, or an isothermal amplification technique.

In some embodiments, the plurality of sequence reads overlap one or more gene loci within a subgenomic interval in the sample. In some embodiments, the one or more gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some embodiments, the disclosed methods may be used to identify variants in the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFIl, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene locus, or any combination thereof.

In some instances, the disclosed methods may be used to identify variants in the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-10, IL-6, IL-6R, JAK1, JAK2, JAK3, KIT, KRAS, MEK, MET, MSI-H, mTOR, PARP, PD-1, PDGFR, PDGFRa, PDGFRβ, PD-L1, PI3Kδ, PIGF, PTCH, RAF, RANKL, RET, ROS1, SLAMF7, VEGF, VEGFA, or VEGFB gene locus, or any combination thereof.

In some embodiments, the plurality of sequence reads overlap one or more gene loci within a subgenomic interval in the sample. In some embodiments, the one or more gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci. In some embodiments, the one or more short variants comprise ALOX12B T402T, ATRX K2225del, CTNNA1 D362N, FLT3 S102S, JAK2 I289V, KEAP1 K97N, KRAS Q61H, MSH3 R573fs*4, PDK1 T306M, STAG2 C527F, STK11 649_650delCC, TSC2 A68S, or any combination thereof. In some embodiments, the one or more cTMB-qualified short variants comprise CTNNA1 D362N, FLT3 S102S, STAG2 C527F, TSC2 A68S, or any combination thereof.

In some embodiments, the one or more cTMB-qualified short variants comprise CTNNA1 D362N. Catenin alpha-1 (CTNNA1; UniProt #P35221) is a protein that associates with the cytoplasmic domain of a variety of cadherins to form a complex which is linked to the actin filament network. CTNNA1 may play a crucial role in cell differentiation. In some embodiments, the one or more cTMB-qualified short variants comprise FLT3 S102S. Receptor-type tyrosine-protein kinase (FLT3; UniProt #P36888) is a tyrosine-protein kinase that helps to regulate differentiation, proliferation and survival of hematopoietic progenitor cells and dendritic cells. In some embodiments, the one or more cTMB-qualified short variants comprise STAG2 C527F. Cohesin subunit SA-2 (STAG2; UniProt #Q8N3U4) is a component of cohesin complex, which is required for the cohesion of sister chromatids after DNA replication. In some embodiments, the one or more cTMB-qualified short variants comprise TSC2 A68S. Tuberin (TSC2; UniProt #P49815), in complex with TSC1, functions as a tumor suppressor that regulate mTORC1 signaling. In some embodiments, the one or more cTMB-qualified short variants comprise CTNNA1 D362N, FLT3 S102S, STAG2 C527F, TSC2 A68S, or any combination thereof.

In some embodiments, the method further comprises generating, by the one or more processors, a report comprising a cTMB for the subject. In some embodiments, the method further comprises transmitting the report to a healthcare provider. In some embodiments, the report is transmitted via a computer network or a peer-to-peer connection.

### III. METHODS OF TREATMENT

In some aspects, provided herein is a method of treating a subject having a cancer comprising determining a clonal tumor mutational burden (cTMB) value in a sample from the subject by the method of any method described herein. In some embodiments, the method further comprises treating the subject with an immuno-oncology (IO) therapy if the cTMB value (for example, a cTMB score) determined for the sample is at or above a threshold cTMB value (or threshold cTMB score).

In some embodiments, the method of treating a subject having a cancer comprises determining a clonal tumor mutational burden (cTMB) value in a sample from the subject. In some embodiments, determining the cTMB comprises providing a plurality of nucleic acid molecules obtained from a sample from a subject. In some embodiments, the method further comprises ligating one or more adapters onto one or more nucleic acid molecules from the plurality of nucleic acid molecules. In some embodiments, the method further comprises amplifying the one or more ligated nucleic acid molecules from the plurality of nucleic acid molecules. In some embodiments, the method further comprises capturing amplified nucleic acid molecules from the amplified nucleic acid molecules. In some embodiments, the method further comprises sequencing, by a sequencer, the captured nucleic acid molecules to obtain a plurality of sequence reads that represent the captured nucleic acid molecules. In some embodiments, the method further comprises receiving, at one or more processors, sequence read data for the plurality of sequence reads. In some embodiments, the method further comprises identifying, using the one or more processors, one or more short variants from the sequence read data. In some embodiments, the method further comprises filtering, using the one or more processors, the one or more short variants to select one or more clonal tumor mutational burden (cTMB)-qualified short variants, wherein the filtering comprises excluding short variants identified as subclonal based on a tumor amount measure for a respective short variant. For example, the tumor amount measure for a respective short variant may comprise a cancer cell fraction (CCF) of the variant or a variant allele frequency (VAF). In some embodiments, the method further comprises determining, using the one or more processors, a cTMB value (for example, a cTMB score) for the sample based on the number of cTMB-qualified short variants. In some embodiments, the method further comprises treating the subject with an immuno-oncology (IO) therapy if the cTMB value determined for the sample is at or above a threshold cTMB value (or threshold cTMB score).

In some embodiments, the method of treating a subject having a cancer comprises determining a clonal tumor mutational burden (cTMB) value in a sample from the subject. In some embodiments, determining the cTMB comprises providing a plurality of nucleic acid molecules obtained from a sample from a subject. In some embodiments, the method further comprises ligating one or more adapters onto one or more nucleic acid molecules from the plurality of nucleic acid molecules. In some embodiments, the method further comprises amplifying the one or more ligated nucleic acid molecules from the plurality of nucleic acid molecules. In some embodiments, the method further comprises capturing amplified nucleic acid molecules from the amplified nucleic acid molecules. In some embodiments, the method further comprises sequencing, by a sequencer, the captured nucleic acid molecules to obtain a plurality of sequence reads that represent the captured nucleic acid molecules. In some embodiments, the method further comprises receiving, at one or more processors, genomic data for a subject, where the genomic data indicates a presence or absence of one or more short variants in a sample derived from the subject. In some embodiments, the method further comprises identifying, using the one or more processors, one or more short variants from the sequence read data. In some embodiments, the method further comprises filtering, using the one or more processors, the one or more short variants to select one or more clonal tumor mutational burden (cTMB)-qualified short variants, wherein the filtering comprises excluding short variants identified as subclonal based on a tumor amount measure for a respective short variant. For example, the tumor amount measure for a respective short variant may comprise a cancer cell fraction (CCF) of the variant or a variant allele frequency (VAF). In some embodiments, the method further comprises determining, using the one or more processors, a clonal tumor mutational burden (cTMB) for the sample based on the number of cTMB-qualified short variants. In some embodiments, the method further comprises treating the subject with an immuno-oncology (IO) therapy if the cTMB value (for example, a cTMB score) determined for the sample is at or above a threshold cTMB value (or threshold cTMB score).

In some aspects, provided herein is a method of selecting a treatment for a subject having a cancer, the method comprising determining a clonal tumor mutational burden (cTMB) value in a sample from the subject by the method of any method described herein. In some embodiments, if the cTMB value (for example, a cTMB score) determined for the sample is at or above a threshold cTMB value (or threshold cTMB score), the subject is identified as one who may benefit from treatment with an immune-oncology (IO) therapy.

In some embodiments, the method of selecting a treatment for a subject having a cancer comprises determining a clonal tumor mutational burden (cTMB) value in a sample from the subject. In some embodiments, determining the cTMB comprises providing a plurality of nucleic acid molecules obtained from a sample from a subject. In some embodiments, the method further comprises ligating one or more adapters onto one or more nucleic acid molecules from the plurality of nucleic acid molecules. In some embodiments, the method further comprises amplifying the one or more ligated nucleic acid molecules from the plurality of nucleic acid molecules. In some embodiments, the method further comprises capturing amplified nucleic acid molecules from the amplified nucleic acid molecules. In some embodiments, the method further comprises sequencing, by a sequencer, the captured nucleic acid molecules to obtain a plurality of sequence reads that represent the captured nucleic acid molecules. In some embodiments, the method further comprises receiving, at one or more processors, sequence read data for the plurality of sequence reads. In some embodiments, the method further comprises identifying, using the one or more processors, one or more short variants from the sequence read data. In some embodiments, the method further comprises filtering, using the one or more processors, the one or more short variants to select one or more clonal tumor mutational burden (cTMB)-qualified short variants, wherein the filtering comprises excluding short variants identified as subclonal based on a tumor amount measure for a respective short variant. For example, the tumor amount measure for a respective short variant may comprise a cancer cell fraction (CCF) of the variant or a variant allele frequency (VAF). In some embodiments, the method further comprises determining, using the one or more processors, a cTMB value (for example, a cTMB score) for the sample based on the number of cTMB-qualified short variants. In some embodiments, if the cTMB value determined for the sample is at or above a threshold cTMB value (or cTMB score), the subject is identified as one who may benefit from treatment with an immune-oncology (IO) therapy.

In some embodiments, the method of selecting a treatment for a subject having a cancer comprises determining a clonal tumor mutational burden (cTMB) value in a sample from the subject. In some embodiments, determining the cTMB comprises providing a plurality of nucleic acid molecules obtained from a sample from a subject. In some embodiments, the method further comprises ligating one or more adapters onto one or more nucleic acid molecules from the plurality of nucleic acid molecules. In some embodiments, the method further comprises amplifying the one or more ligated nucleic acid molecules from the plurality of nucleic acid molecules. In some embodiments, the method further comprises capturing amplified nucleic acid molecules from the amplified nucleic acid molecules. In some embodiments, the method further comprises sequencing, by a sequencer, the captured nucleic acid molecules to obtain a plurality of sequence reads that represent the captured nucleic acid molecules. In some embodiments, the method further comprises receiving, at one or more processors, genomic data for a subject, where the genomic data indicates a presence or absence of one or more short variants in a sample derived from the subject. In some embodiments, the method further comprises identifying, using the one or more processors, one or more short variants from the sequence read data. In some embodiments, the method further comprises filtering, using the one or more processors, the one or more short variants to select one or more clonal tumor mutational burden (cTMB)-qualified short variants, wherein the filtering comprises excluding short variants identified as subclonal based on a tumor amount measure for a respective short variant. For example, the tumor amount measure for a respective short variant may comprise a cancer cell fraction (CCF) of the variant or a variant allele frequency (VAF). In some embodiments, the method further comprises determining, using the one or more processors, a cTMB value (for example, a cTMB score) for the sample based on the number of cTMB-qualified short variants. In some embodiments, if the cTMB value determined for the sample is at or above a threshold cTMB value (or threshold cTMB score), the subject is identified as one who may benefit from treatment with an immune-oncology (IO) therapy.

In some aspects, provided herein is a method of identifying a subject having a cancer for treatment with an immune-oncology (IO) therapy comprising determining a clonal tumor mutational burden (cTMB) value in a sample from the subject by the method of any method described herein. In some embodiments, the method further comprises identifying the subject for treatment with the IO therapy if the cTMB value (for example, a cTMB score) determined for the sample is at or above a threshold cTMB value (or threshold cTMB score).

In some embodiments, the method of identifying a subject having a cancer for treatment with an immune-oncology (IO) therapy comprises determining a clonal tumor mutational burden (cTMB) value in a sample from the subject. In some embodiments, determining the cTMB comprises providing a plurality of nucleic acid molecules obtained from a sample from a subject. In some embodiments, the method further comprises ligating one or more adapters onto one or more nucleic acid molecules from the plurality of nucleic acid molecules. In some embodiments, the method further comprises amplifying the one or more ligated nucleic acid molecules from the plurality of nucleic acid molecules. In some embodiments, the method further comprises capturing amplified nucleic acid molecules from the amplified nucleic acid molecules. In some embodiments, the method further comprises sequencing, by a sequencer, the captured nucleic acid molecules to obtain a plurality of sequence reads that represent the captured nucleic acid molecules. In some embodiments, the method further comprises receiving, at one or more processors, sequence read data for the plurality of sequence reads. In some embodiments, the method further comprises identifying, using the one or more processors, one or more short variants from the sequence read data. In some embodiments, the method further comprises filtering, using the one or more processors, the one or more short variants to select one or more clonal tumor mutational burden (cTMB)-qualified short variants, wherein the filtering comprises excluding short variants identified as subclonal based on a tumor amount measure for a respective short variant. For example, the tumor amount measure for a respective short variant may comprise a cancer cell fraction (CCF) of the variant or a variant allele frequency (VAF). In some embodiments, the method further comprises determining, using the one or more processors, a cTMB value (for example, a cTMB score) for the sample based on the number of cTMB-qualified short variants. In some embodiments, the method further comprises identifying the subject for treatment with the IO therapy if the cTMB value determined for the sample is at or above a threshold cTMB value (or threshold cTMB score).

In some embodiments, the method of identifying a subject having a cancer for treatment with an immune-oncology (IO) therapy comprises determining a clonal tumor mutational burden (cTMB) value in a sample from the subject. In some embodiments, determining the cTMB comprises providing a plurality of nucleic acid molecules obtained from a sample from a subject. In some embodiments, the method further comprises ligating one or more adapters onto one or more nucleic acid molecules from the plurality of nucleic acid molecules. In some embodiments, the method further comprises amplifying the one or more ligated nucleic acid molecules from the plurality of nucleic acid molecules. In some embodiments, the method further comprises capturing amplified nucleic acid molecules from the amplified nucleic acid molecules. In some embodiments, the method further comprises sequencing, by a sequencer, the captured nucleic acid molecules to obtain a plurality of sequence reads that represent the captured nucleic acid molecules. In some embodiments, the method further comprises receiving, at one or more processors, genomic data for a subject, where the genomic data indicates a presence or absence of one or more short variants in a sample derived from the subject. In some embodiments, the method further comprises identifying, using the one or more processors, one or more short variants from the sequence read data. In some embodiments, the method further comprises filtering, using the one or more processors, the one or more short variants to select one or more clonal tumor mutational burden (cTMB)-qualified short variants, wherein the filtering comprises excluding short variants identified as subclonal based on a tumor amount measure for a respective short variant. For example, the tumor amount measure for a respective short variant may comprise a cancer cell fraction (CCF) of the variant or a variant allele frequency (VAF). In some embodiments, the method further comprises determining, using the one or more processors, a cTMB value (for example, a cTMB score) for the sample based on the number of cTMB-qualified short variants. In some embodiments, the method further comprises identifying the subject for treatment with the IO therapy if the cTMB value determined for the sample is at or above a threshold cTMB value (or threshold cTMB score).

In some aspects, provided herein is a method of identifying one or more treatment options for a subject having a cancer, the method comprising determining a clonal tumor mutational burden (cTMB) value in a sample from the subject by the method of any method described herein. In some embodiments, the method further comprises generating a report comprising one or more treatment options identified for the subject based at least in part on the cTMB value determined for the sample. In some embodiments, if the cTMB value is at or above a threshold cTMB value (or threshold cTMB score), the subject is identified as one who may benefit from treatment with an immune-oncology (IO) therapy.

In some aspects, provided herein is a method of predicting survival of a subject having cancer, the method comprising determining a clonal tumor mutational burden (cTMB) value in a sample from the subject by the method of method described herein. In some embodiments, if the cTMB value determined for the sample is at or above a threshold cTMB value (or threshold cTMB score), the subject is predicted to have longer survival when treated with an immune-oncology (IO) therapy. In some embodiments, if the cTMB value determined for the sample is below the threshold cTMB value (or threshold cTMB score), the subject is predicted to have shorter survival when treated with an immune-oncology (IO) therapy. In some embodiments, if the cTMB value determined for the sample is at or above a threshold cTMB value (or threshold cTMB score), the subject is predicted to have longer survival when treated with an immune-oncology (IO) therapy, as compared to a subject determined to have a cTMB value below the threshold cTMB value (or threshold cTMB score).

In some embodiments, the method of predicting survival of a subject having cancer, the method comprising determining a clonal tumor mutational burden (cTMB) value (for example, a cTMB score) in a sample from the subject. In some embodiments, determining the cTMB comprises providing a plurality of nucleic acid molecules obtained from a sample from a subject. In some embodiments, the method further comprises ligating one or more adapters onto one or more nucleic acid molecules from the plurality of nucleic acid molecules. In some embodiments, the method further comprises amplifying the one or more ligated nucleic acid molecules from the plurality of nucleic acid molecules. In some embodiments, the method further comprises capturing amplified nucleic acid molecules from the amplified nucleic acid molecules. In some embodiments, the method further comprises sequencing, by a sequencer, the captured nucleic acid molecules to obtain a plurality of sequence reads that represent the captured nucleic acid molecules. In some embodiments, the method further comprises receiving, at one or more processors, sequence read data for the plurality of sequence reads. In some embodiments, the method further comprises identifying, using the one or more processors, one or more short variants from the sequence read data. In some embodiments, the method further comprises filtering, using the one or more processors, the one or more short variants to select one or more clonal tumor mutational burden (cTMB)-qualified short variants, wherein the filtering comprises excluding short variants identified as subclonal based on a tumor amount measure for a respective short variant. For example, the tumor amount measure for a respective short variant may comprise a cancer cell fraction (CCF) of the variant or a variant allele frequency (VAF). In some embodiments, the method further comprises determining, using the one or more processors, a cTMB value (for example, a cTMB score) for the sample based on the number of cTMB-qualified short variants. In some embodiments, if the cTMB value determined for the sample is at or above a threshold cTMB value (or threshold cTMB score), the subject is predicted to have longer survival when treated with an immune-oncology (IO) therapy. In some embodiments, if the cTMB value determined for the sample is below the threshold cTMB value (or threshold cTMB score), the subject is predicted to have shorter survival when treated with an immune-oncology (IO) therapy. In some embodiments, if the cTMB value determined for the sample is at or above a threshold cTMB value (or threshold cTMB score), the subject is predicted to have longer survival when treated with an immune-oncology (IO) therapy, as compared to a subject determined to have a cTMB value below the threshold cTMB value (or threshold cTMB score).

In some embodiments, the method of predicting survival of a subject having cancer, the method comprising determining a clonal tumor mutational burden (cTMB) value in a sample from the subject. In some embodiments, determining the cTMB comprises providing a plurality of nucleic acid molecules obtained from a sample from a subject. In some embodiments, the method further comprises ligating one or more adapters onto one or more nucleic acid molecules from the plurality of nucleic acid molecules. In some embodiments, the method further comprises amplifying the one or more ligated nucleic acid molecules from the plurality of nucleic acid molecules. In some embodiments, the method further comprises capturing amplified nucleic acid molecules from the amplified nucleic acid molecules. In some embodiments, the method further comprises sequencing, by a sequencer, the captured nucleic acid molecules to obtain a plurality of sequence reads that represent the captured nucleic acid molecules. In some embodiments, the method further comprises receiving, at one or more processors, genomic data for a subject, where the genomic data indicates a presence or absence of one or more short variants in a sample derived from the subject. In some embodiments, the method further comprises identifying, using the one or more processors, one or more short variants from the sequence read data. In some embodiments, the method further comprises filtering, using the one or more processors, the one or more short variants to select one or more clonal tumor mutational burden (cTMB)-qualified short variants, wherein the filtering comprises excluding short variants identified as subclonal based on a tumor amount measure for a respective short variant. For example, the tumor amount measure for a respective short variant may comprise a cancer cell fraction (CCF) of the variant or a variant allele frequency (VAF). In some embodiments, the method further comprises determining, using the one or more processors, a cTMB value (for example, a cTMB score) for the sample based on the number of cTMB-qualified short variants. In some embodiments, if the cTMB value determined for the sample is at or above a threshold cTMB value (or threshold cTMB score), the subject is predicted to have longer survival when treated with an immune-oncology (IO) therapy. In some embodiments, if the cTMB value determined for the sample is below the threshold cTMB value (or threshold cTMB score), the subject is predicted to have shorter survival when treated with an immune-oncology (IO) therapy. In some embodiments, if the cTMB value determined for the sample is at or above a threshold cTMB value (or threshold cTMB score), the subject is predicted to have longer survival when treated with an immune-oncology (IO) therapy, as compared to a subject determined to have a cTMB value below the threshold cTMB value (or threshold cTMB score).

In some embodiments, the threshold cTMB value (or threshold cTMB score) is at least about 1 to 100 mutations/Mb, about 2 to 90 mutations/Mb, 3 to 80 mutations/Mb, 5 to 70 mutations/Mb, 10 to 60 mutations/Mb, 20 to 50 mutations/Mb, or 30 to 40 mutations/Mb. In some embodiments, the threshold cTMB value (or threshold cTMB score) is less than less than 2 mutations/Mb, 3 mutations/Mb, less than 5 mutations/Mb, 10 mutations/Mb, 20 mutations/Mb, 30 mutations/Mb, 50 mutations/Mb, 75 mutations/Mb, or 100 mutations/Mb. In some embodiments, the threshold cTMB value (or threshold cTMB score) is at least about 1 mutations/Mb, at least about 2 mutations/Mb, at least about 3 mutations/Mb, at least about 5 mutations/Mb, at least about 10 mutations/Mb, at least about 20 mutations/Mb, at least about 30 mutations/Mb, at least about 40 mutations/Mb, at least about 50 mutations/Mb, at least about 60 mutations/Mb, at least about 70 mutations/Mb, at least about 80 mutations/Mb, at least about 90 mutations/Mb, or at least about 100 mutations/Mb. In some embodiments, the threshold cTMB value (or threshold cTMB score) is at least about 5 mutations/Mb, at least about 6 mutations/Mb, at least 7 mutations/Mb, at least about 8 mutations/Mb, at least about 9 mutations/Mb, at least about 10 mutations/Mb, at least about 11 mutations/Mb, at least about 12 mutations/Mb, at least about 13 mutations/Mb, at least about 14 mutations/Mb, at least about 15 mutations/Mb, at least about 16 mutations/Mb, at least about 17 mutations/Mb, at least about 18 mutations/Mb, at least about 19 mutations/Mb, at least about 20 mutations/Mb, at least about 21 mutations/Mb, at least about 22 mutations/Mb, at least about 23 mutations/Mb, at least about 24 mutations/Mb, or at least about 25 mutations/Mb. In some embodiments, the threshold cTMB value (or threshold cTMB score) is related to the sample from the subject, for example, the tumor type of the sample from the subject. Tumor specific biology can influence the threshold cTMB value, where the threshold cTMB value for a first tumor type may not be the same as the threshold cTMB value for a second tumor type. In some embodiments, the sample from the subject may be a tumor sample from a subject having cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like. For example, the cTMB threshold for a sample from a patient having breast cancer may be different than the cTMB threshold for a sample from a patient having non-small cell lung carcinoma.

In some embodiments, the threshold cTMB value (or threshold cTMB score) is at least about 4 to 100 mutations/Mb, about 4 to 30 mutations/Mb, 8 to 100 mutations/Mb, 8 to 30 mutations/Mb, 10 to 20 mutations/Mb, less than 4 mutations/Mb, or less than 8 mutations/Mb. In some embodiments, the threshold cTMB value (or threshold cTMB score) is at least about 5 mutations/Mb, at least about 10 mutations/Mb, at least about 12 mutations/Mb, at least about 16 mutations/Mb, at least about 20 mutations/Mb, or at least about 30 mutations/Mb. In some embodiments, the threshold cTMB value (or threshold cTMB score) is at least about 7.5 mutations/Mb, at least about 8.5 mutations/Mb, at least about 9.5 mutations/Mb, at least about 11 mutations/Mb, at least about 13.5 mutations/Mb, at least about 16.5 mutations/Mb, at least about 18.5 mutations/Mb, at least about 19 mutations/Mb, or at least about 25 mutations/Mb.

In some embodiments, the immune-oncology (IO) therapy of the method described herein comprises a single IO agent or multiple IO agents. In some embodiments, the IO therapy comprises an immune checkpoint inhibitor. In some embodiments, the immune checkpoint inhibitor comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a cellular therapy, a treatment for cancer being tested in a clinical trial, an immunotherapy, or any combination thereof.

In some embodiments, the immuno-oncology (IO) therapy comprises an immune checkpoint inhibitor, for example, an immune checkpoint inhibitor targeting PD-1, PD-L1, CTLA-4, or a combination thereof. In some embodiments, the immune checkpoint inhibitor is a PD-1 inhibitor. In some embodiments, the immune checkpoint inhibitor comprises one or more of nivolumab, pembrolizumab, cemiplimab, or dostarlimab. In some embodiments, the immune checkpoint inhibitor is a PD-L1-inhibitor. In some embodiments, the immune checkpoint inhibitor comprises one or more of atezolizumab, avelumab, or durvalumab. In some embodiments, the immune checkpoint inhibitor is a CTLA-4 inhibitor. In some embodiments, the CTLA-4 inhibitor comprises ipilimumab.

In some embodiments, the immune checkpoint inhibitor comprises a nucleic acid. In some embodiments, the nucleic acid comprises a double-stranded RNA (dsRNA), a small interfering RNA (siRNA), or a small hairpin RNA (shRNA).

In some embodiments, the immune checkpoint inhibitor comprises a cellular therapy. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In some embodiments, the method further comprises treating the subject with the immune-oncology (IO) therapy. In some embodiments, the method further comprises treating the subject with an additional anti-cancer therapy. In some embodiments, the additional anti-cancer therapy comprises one or more of a small molecule inhibitor, a chemotherapeutic agent, a cancer a targeted therapy (e.g., immunotherapy), an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

In some embodiments, the chemotherapeutic agent comprises one or more of an alkylating agent, an alkyl sulfonates aziridine, an ethylenimine, a methylamelamine, an acetogenin, a camptothecin, a bryostatin, a callystatin, CC-1065, a cryptophycin, aa dolastatin, a duocarmycin, a eleutherobin, a pancratistatin, a sarcodictyin, a spongistatin, a nitrogen mustard, a nitrosureas, an antibiotic, a dynemicin, a bisphosphonate, an esperamicina a neocarzinostatin chromophore or a related chromoprotein enediyne antibiotic chromophore, an anti-metabolite, a folic acid analogue, a purine analog, a pyrimidine analog, an androgens, an anti-adrenal, a folic acid replenisher, aldophosphamide glycoside, aminolevulinic acid, eniluracil, amsacrine, bestrabucil, bisantrene, edatraxate, defofamine, demecolcine, diaziquone, elformithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, a PSK polysaccharide complex, razoxane, rhizoxin, sizofiran, spirogermanium, tenuazonic acid, triaziquone, 2,2′,2″-trichlorotriethylamine, a trichothecene, urethan, vindesine, dacarbazine, mannomustine, mitobronitol, mitolactol, pipobroman, gacytosine, arabinoside (“Ara-C”), cyclophosphamide, a taxoid, 6-thioguanine, mercaptopurine, a platinum coordination complex, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluorometlhylomithine (DMFO), a retinoid, capecitabine, carboplatin, procarbazine, plicomycin, gemcitabine, navelbine, farnesyl-protein transferase inhibitors, transplatinum, or any combination thereof.

In some embodiments, the targeted therapy (or anti-cancer target therapy) may comprise abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aligopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane 1131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some embodiments, the cancer is a B cell cancer, a melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer or carcinoma, lung non-small cell lung carcinoma (NSCLC), head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the subject is a human. In some embodiments, the subject has previously been treated with an anti-cancer therapy. In some embodiments, the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof. In some embodiments, the subject has not previously been treated with an anti-cancer therapy.

### IV. METHODS OF USE

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for determining clonal tumor mutational burden (cTMB) may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alterations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for determining clonal tumor mutational burden (cTMB) may be used to select a subject (e.g., a patient) for a clinical trial based on the cTMB threshold value determined for one or more gene loci. In some instances, patient selection for clinical trials based on, e.g., identification of the cTMB threshold value for one or more gene loci, may accelerate the development of targeted therapies and improve the healthcare outcomes for treatment decisions. In some instances, the disclosed methods for determining clonal tumor mutational burden (cTMB) may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

In some instances, the disclosed methods for determining clonal tumor mutational burden (cTMB) may be used in treating a disease (e.g., a cancer) in a subject. For example, in response to determining clonal tumor mutational burden (cTMB) using any of the methods disclosed herein, an effective amount of an anti-cancer therapy or anti-cancer treatment may be administered to the subject.

In some instances, the disclosed methods for determining clonal tumor mutational burden (cTMB) may be used for monitoring disease progression or recurrence (e.g., cancer or tumor progression or recurrence) in a subject. For example, in some instances, the methods may be used to determine cTMB in a first sample obtained from the subject at a first time point, and used to determine cTMB in a second sample obtained from the subject at a second time point, where comparison of the first determination of cTMB and the second determination of cTMB allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the determination of clonal tumor mutational burden (cTMB).

In some instances, the value of clonal tumor mutational burden (cTMB) determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for determining clonal tumor mutational burden (cTMB) may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through, for example, comprehensive genomic profiling (CGP), which is a next-generation sequencing (NGS) approach used to assess hundreds of genes (including relevant cancer biomarkers) in a single assay. Inclusion of the disclosed methods for determining cTMB as part of a genomic profiling process (or inclusion of the output from the disclosed methods for determining cTMB as part of the genomic profile of the subject) can improve the validity of, e.g., disease detection calls and treatment decisions, made on the basis of the genomic profile by, for example, independently confirming the presence of TMB in a given patient sample. In some instances, TMB may be confirmed using CGP, computational pathology derived imaging modalities, or other techniques.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### V. SAMPLES

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### VI. SUBJECTS

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### VII. CANCERS

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### VIII. THERAPIES

Some aspects of the disclosure provide for therapies. In some embodiments, the therapy comprises an immune-oncology (IO) therapy, or an IO therapy in combination with a chemotherapy. In some embodiments, the therapy comprises a targeted therapy. In some embodiments, the therapy comprises an anti-cancer therapy.

### A. Immuno-Oncology Therapies

Certain aspects of the present disclosure relate to immuno-oncology (IO) therapies. In some embodiments the IO therapy comprises an immune checkpoint inhibitor (ICI).

As is known in the art, a checkpoint inhibitor targets at least one immune checkpoint protein to alter the regulation of an immune response. Immune checkpoint proteins include, e.g., CTLA4, PD-L1, PD-1, PD-L2, VISTA, B7-H2, B7-H3, B7-H4, B7-H6, 2B4, ICOS, HVEM, CEACAM, LAIR1, CD80, CD86, CD276, VTCN1, MHC class I, MHC class II, GALS, adenosine, TGFR, CSF1R, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some embodiments, molecules involved in regulating immune checkpoints include, but are not limited to: PD-1 (CD279), PD-L1 (B7-H1, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GAL9, VISTA (PD-1H), TIGIT, LAIR1, CD160, 2B4, TGFRbeta, A2AR, GITR (CD357), CD80 (B7-1), CD86 (B7-2), CD276 (B7-H3), VTCNI (B7-H4), MHC class I, MHC class II, GALS, adenosine, TGFR, B7-H1, OX40 (CD134), CD94 (KLRD1), CD137 (4-1BB), CD137L (4-1BBL), CD40, IDO, CSF1R, CD40L, CD47, CD70 (CD27L), CD226, HHLA2, ICOS (CD278), ICOSL (CD275), LIGHT (TNFSF14, CD258), NKG2a, NKG2d, OX40L (CD134L), PVR (NECL5, CD155), SIRPa, MICA/B, and/or arginase. In some embodiments, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. Examples of checkpoint inhibitors include, without limitation, a PD-1 axis binding antagonist, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or antibodies, such as human antibodies (see, e.g., International Patent Publication WO2015016718; Pardoll, The blockade of immune checkpoints in cancer immunotherapy, Nat Rev Cancer, vol. 12, no. 4, pp. 252-64 (2012); both incorporated herein by reference). In some embodiments, known inhibitors of immune checkpoint proteins or analogs thereof may be used, in particular chimerized, humanized or human forms of antibodies may be used.

In some embodiments according to any of the embodiments described herein, the immune checkpoint inhibitor comprises a PD-1 antagonist/inhibitor or a PD-L1 antagonist/inhibitor.

In some embodiments, the checkpoint inhibitor is a PD-L1 axis binding antagonist, e.g., a PD-1 binding antagonist, a PD-L1 binding antagonist, or a PD-L2 binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “B7-H,” and “PDL1.” An exemplary human PD-L1 is shown in UniProtKB/Swiss-Prot Accession No. Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some instances, the PD-1 binding antagonist/inhibitor is a molecule that inhibits the binding of PD-1 to its ligand binding partners. In a specific embodiment, the PD-1 ligand binding partners are PD-L1 and/or PD-L2. In another instance, a PD-L1 binding antagonist/inhibitor is a molecule that inhibits the binding of PD-L1 to its binding ligands. In a specific embodiment, PD-L1 binding partners are PD-1 and/or B7-1. In another instance, the PD-L2 binding antagonist is a molecule that inhibits the binding of PD-L2 to its ligand binding partners. In a specific embodiment, the PD-L2 binding ligand partner is PD-1. The antagonist may be an antibody, an antigen binding fragment thereof, an immunoadhesin, a fusion protein, or an oligopeptide. In some embodiments, the PD-1 binding antagonist is a small molecule, a nucleic acid, a polypeptide (e.g., antibody), a carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is MDX-1 106 (nivolumab), MK-3475 (pembrolizumab, Keytruda®), cemiplimab, dostarlimab, MEDI-0680 (AMP-514), PDR001, REGN2810, MGA- 012, JNJ-63723283, BI 754091, or BGB-108. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some instances, the PD-1 binding antagonist is AMP-224. Other examples of anti-PD-1 antibodies include, but are not limited to, MEDI-0680 (AMP-514; AstraZeneca), PDR001 (CAS Registry No. 1859072-53-9; Novartis), REGN2810 (LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANB011; Tesaro/AnaptysBio), AM0001 (ARMO Biosciences), ENUM 244C8 (Enumeral Biomedical Holdings), or ENUM 388D4 (Enumeral Biomedical Holdings). In some embodiments, the PD-1 axis binding antagonist comprises tislelizumab (BGB-A317), BGB-108, STI-A1110, AM0001, BI 754091, sintilimab (IBI308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, Keytruda®), PF-06801591, cemiplimab (REGN-2810, REGEN2810), dostarlimab (TSR-042, ANB011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, or CCX-4503, or derivatives thereof.

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In some embodiments, the PD-L1 binding antagonist is an anti-PD-L1 antibody. In some embodiments, the anti-PD-L1 antibody can bind to a human PD-L1, for example a human PD-L1 as shown in UniProtKB/Swiss-Prot Accession No. Q9NZQ7.1, or a variant thereof. In some embodiments, the PD-L1 binding antagonist is a small molecule, a nucleic acid, a polypeptide (e.g., antibody), a carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting the binding between PD-L1 and PD-1, and/or between PD-L1 and B7-1. In some instances, the anti-PD-L1 antibody is a monoclonal antibody. In some instances, the anti-PD-L1 antibody is an antibody fragment selected from a Fab, Fab′-SH, Fv, scFv, or (Fab′)2 fragment. In some instances, the anti-PD-L1 antibody is a humanized antibody. In some instances, the anti-PD-L1 antibody is a human antibody. In some instances, the anti-PD-L1 antibody is selected from YW243.55.S70, MPDL3280A (atezolizumab), MDX-1 105, MEDI4736 (durvalumab), or MSB0010718C (avelumab). In some embodiments, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, or FS-118, or a derivative thereof.

In some embodiments, the checkpoint inhibitor is an antagonist/inhibitor of CTLA4. In some embodiments, the checkpoint inhibitor is a small molecule antagonist of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist is a small molecule, a nucleic acid, a polypeptide (e.g., antibody), a carbohydrate, a lipid, a metal, or a toxin. In some embodiments, the CTLA-4 inhibitor comprises ipilimumab (IBI310, BMS-734016, MDX010, MDX-CTLA4, MEDI4736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof.

In some embodiments, the anti-PD-1 antibody or antibody fragment is MDX-1106 (nivolumab), MK-3475 (pembrolizumab, Keytruda®), cemiplimab, dostarlimab, MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the PD-1 binding antagonist is an anti-PD-1 immunoadhesin. In some embodiments, the anti-PD-1 immunoadhesin is AMP-224. In some embodiments, the anti-PD-L1 antibody or antibody fragment is YW243.55.S70, MPDL3280A (atezolizumab), MDX-1105, MEDI4736 (durvalumab), MSB0010718C (avelumab), LY3300054, STI-A1014, KN035, FAZ053, or CX-072.

In some embodiments, the immune checkpoint inhibitor comprises a LAG-3 inhibitor (e.g., an antibody, an antibody conjugate, or an antigen-binding fragment thereof). In some embodiments, the LAG-3 inhibitor comprises a small molecule, a nucleic acid, a polypeptide (e.g., an antibody), a carbohydrate, a lipid, a metal, or a toxin. In some embodiments, the LAG-3 inhibitor comprises a small molecule. In some embodiments, the LAG-3 inhibitor comprises a LAG-3 binding agent. In some embodiments, the LAG-3 inhibitor comprises an antibody, an antibody conjugate, or an antigen-binding fragment thereof. In some embodiments, the LAG-3 inhibitor comprises eftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceding.

In some embodiments, the immune checkpoint inhibitor is monovalent and/or monospecific. In some embodiments, the immune checkpoint inhibitor is multivalent and/or multispecific.

In some embodiments, the immune checkpoint inhibitor may be administered in combination with an immunoregulatory molecule or a cytokine. An immunoregulatory profile is required to trigger an efficient immune response and balance the immunity in a subject. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, gIp10, TCA-3, MCP-1, MIF, MIP-1α, MIP-1β, Rantes, macrophage colony stimulating factor (M-CSF), granulocyte colony stimulating factor (G-CSF), or granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present disclosure. Examples of chemokines include, but are not limited to, MIP-3α (Lax), MIP-3β, Hcc-1, MPIF-1, MPIF-2, MCP-2, MCP-3, MCP-4, MCP-5, Eotaxin, Tarc, Elc, 1309, IL-8, GCP-2 Groa, Gro-P, Nap-2, Ena-78, Ip-10, MIG, I-Tac, SDF-1, or BCA-1 (Blc), as well as functional fragments thereof. In some embodiments, the immunoregulatory molecule is included with any of the treatments provided herein.

In some embodiments, the immune checkpoint inhibitor is a first line immune checkpoint inhibitor. In some embodiments, the immune checkpoint inhibitor is a second line immune checkpoint inhibitor. In some embodiments, an immune checkpoint inhibitor is administered in combination with one or more additional anti-cancer therapies or treatments.

In some embodiments, the methods of the disclosure further comprise treating an individual with the IO therapy. In some embodiments, an IO therapy is administered as a monotherapy. In some embodiments, the IO therapy comprises one or multiple IO agents.

In some embodiments, the individual is treated with an IO therapy in combination with a second therapy. In some embodiments, the individual with the IO therapy in combination with a chemotherapy. In some embodiments, the IO therapy and the chemotherapy are administered concurrently or sequentially.

### B. Chemotherapies

Certain aspects of the present disclosure relate to chemotherapies.

In some embodiments, the methods provided herein comprise administering to an individual a chemotherapy, e.g., in combination with another anti-cancer therapy of the disclosure, such as an IO therapy. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antibiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabine, navelbine, famesyl-protein transferase inhibitors, transplatinum, and pharmaceutically acceptable salts, acids, or derivatives of any of the above.

Some non-limiting examples of chemotherapeutic drugs which can be combined with anti-cancer therapies of the present disclosure, such as an IO therapy, are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).

### C. Targeted Therapies

Certain aspects of the disclosure provide for targeted therapies.

In some embodiments, the targeted therapy is a clonal tumor mutational burden (cTMB)-targeted therapy. In some embodiments, the cTMB-targeted therapy comprises an immune checkpoint inhibitor. In some embodiments, the immune checkpoint inhibitor is an anti-PD1 therapy or an anti-PD-L1 therapy. In some embodiments, the anti-PD-1 therapy comprises one or more of nivolumab, pembrolizumab, cemiplimab, or dostarlimab. In some embodiments, the anti-PD-L1 therapy comprises one or more of atezolizumab, avelumab, or durvalumab. In some embodiments, the cTMB-targeted therapy is administered to an individual having a predetermined cTMB value.

In some embodiments, the targeted therapy is a MSI-high-targeted therapy. In some embodiments, the MSI-high-targeted therapy comprises an immune checkpoint inhibitor. In some embodiments, the immune checkpoint inhibitor is an anti-PD1 therapy, an anti-PD-L1 therapy, or an anti-CTLA-4 therapy. In some embodiments, the anti-PD-1 therapy comprises one or more of nivolumab, pembrolizumab, cemiplimab, or dostarlimab. In some embodiments, the anti-PD-1 therapy comprises one or more of nivolumab, pembrolizumab, cemiplimab, or dostarlimab. In some embodiments, the anti-CTLA-4 therapy comprises ipilimumab In some embodiments, the anti-PD-L1 therapy comprises one or more of atezolizumab, avelumab, or durvalumab. In some embodiments, the MSI-high-targeted therapy is administered to an individual having a MSI-high score.

In some embodiments, the targeted therapy is an HRD-positive targeted therapy.

Treatments that are effective in a HRD positive tumor and may be used as a HRD-positive targeted therapy can include one or more PARP inhibitors and/or one or more platinum-based agents. PARP inhibitors may include, but are not limited to, veliparib, olaparib, talazoparib, iniparib, rucaparib, and niraparib. PARP inhibitors are described in Murphy et al., PARP inhibitors: clinical development, emerging differences, and the current therapeutic issues, Cancer Drug Resist., vol. 2, pp. 665-79 (2019). Platinum-based agents may include, but are not limited to, cisplatin, oxaliplatin, and carboplatin. Platinum-based drugs are described in Rottenberg et al., The rediscovery of platinum-based cancer therapy, Nat. Rev. Cancer, vol. 21, no. 1, pp. 37-50 (2021).

n some embodiments, the HRD-positive targeted therapy is selected from the group consisting of a platinum-based drug and a PARP inhibitor, or any combination thereof. In some embodiments, the PARP inhibitor is olaparib, niraparib, or rucaparib. In some embodiments, the HRD-positive targeted therapy is administered to an individual having an HRD-positive status.

### D. Anti-Cancer Therapies

Certain aspects of the disclosure provide for anti-cancer therapies.

In some embodiments, the anti-cancer therapy comprises a kinase inhibitor. In some embodiments, the methods provided herein comprise administering to the individual a kinase inhibitor, e.g., in combination with another anti-cancer therapy such as an immune checkpoint inhibitor. Examples of kinase inhibitors include those that target one or more receptor tyrosine kinases, e.g., BCR-ABL, B-Raf, EGFR, HER-2/ErbB2, IGF-IR, PDGFR-a, PDGFR-β, cKit, Flt-4, Flt3, FGFR1, FGFR3, FGFR4, CSF1R, c-Met, RON, c-Ret, or ALK; one or more cytoplasmic tyrosine kinases, e.g., c-SRC, c-YES, Abl, or JAK-2; one or more serine/threonine kinases, e.g., ATM, Aurora A & B, CDKs, mTOR, PKCi, PLKs, b-Raf, S6K, or STK11/LKB1; or one or more lipid kinases, e.g., PI3K or SKI. Small molecule kinase inhibitors include PHA-739358, nilotinib, dasatinib, PD166326, NSC 743411, lapatinib (GW-572016), canertinib (CI-1033), semaxinib (SU5416), vatalanib (PTK787/ZK222584), sutent (SU1 1248), sorafenib (BAY 43-9006), or leflunomide (SU101). Additional non-limiting examples of tyrosine kinase inhibitors include imatinib (Gleevec/Glivec) and gefitinib (Iressa).

In some embodiments, the anti-cancer therapy comprises an anti-angiogenic agent. In some embodiments, the methods provided herein comprise administering to the individual an anti-angiogenic agent, e.g., in combination with another anti-cancer therapy such as an immune checkpoint inhibitor. Angiogenesis inhibitors prevent the extensive growth of blood vessels (angiogenesis) that tumors require to survive. Non-limiting examples of angiogenesis-mediating molecules or angiogenesis inhibitors which may be used in the methods of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-β and IFN-7, CXCL10, IL-4, IL-12 and IL-18, prothrombin (kringle domain-2), antithrombin III fragment, prolactin, VEGI, SPARC, osteopontin, maspin, canstatin, proliferin-related protein, restin and drugs such as bevacizumab, itraconazole, carboxyamidotriazole, TNP-470, CM101, IFN-α platelet factor-4, suramin, SU5416, thrombospondin, VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina v 33 inhibitors, linomide, or tasquinimod. In some embodiments, known therapeutic candidates that may be used according to the methods of the disclosure include naturally occurring angiogenic inhibitors, including without limitation, angiostatin, endostatin, or platelet factor-4. In another embodiment, therapeutic candidates that may be used according to the methods of the disclosure include, without limitation, specific inhibitors of endothelial cell growth, such as TNP-470, thalidomide, and interleukin-12. Still other anti-angiogenic agents that may be used according to the methods of the disclosure include those that neutralize angiogenic molecules, including without limitation, antibodies to fibroblast growth factor, antibodies to vascular endothelial growth factor, antibodies to platelet derived growth factor, or antibodies or other types of inhibitors of the receptors of EGF, VEGF or PDGF. In some embodiments, anti-angiogenic agents that may be used according to the methods of the disclosure include, without limitation, suramin and its analogs, and tecogalan. In other embodiments, anti-angiogenic agents that may be used according to the methods of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used according to the methods of the disclosure includes, without limitation, anti-adhesion molecules, such as antibodies to integrin alpha v beta 3. Still other anti-angiogenic compounds or compositions that may be used according to the methods of the disclosure include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound that may be used according to the methods of the disclosure is an antibody to VEGF, such as Avastin®/bevacizumab (Genentech).

In some embodiments, the anti-cancer therapy comprises an anti-DNA repair therapy. In some embodiments, the methods provided herein comprise administering to the individual an anti-DNA repair therapy, e.g., in combination with another anti-cancer therapy such as an immune checkpoint inhibitor. In some embodiments, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026).

In some embodiments, the anti-cancer therapy comprises a radiosensitizer. In some embodiments, the methods provided herein comprise administering to the individual a radiosensitizer, e.g., in combination with another anti-cancer therapy such as an immune checkpoint inhibitor. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors.

In some embodiments, the anti-cancer therapy comprises an anti-inflammatory agent. In some embodiments, the methods provided herein comprise administering to the individual an anti-inflammatory agent, e.g., in combination with another anti-cancer therapy such as an immune checkpoint inhibitor. In some embodiments, the anti-inflammatory agent is an agent that blocks, inhibits, or reduces inflammation or signaling from an inflammatory signaling pathway In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23; interferons (IFNs), e.g., IFNα, IFNβ, IFNγ, IFN-7 inducing factor (IGIF); transforming growth factor-β (TGF-β); transforming growth factor-α (TGF-α); tumor necrosis factors, e.g., TNF-α, TNF-β, TNF-RI, TNF-RII; CD23; CD30; CD40L; EGF; G-CSF; GDNF; PDGF-BB; RANTES/CCL5; IKK; NF-1B; TLR2; TLR3; TLR4; TL5; TLR6; TLR7; TLR8; TLR8; TLR9; and/or any cognate receptors thereof. In some embodiments, the anti-inflammatory agent is an IL-1 or IL-1 receptor antagonist, such as anakinra (Kineret®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (Remicade®), golimumab (Simponi®), adalimumab (Humira®), certolizumab pegol (Cimzia®) or etanercept. In some embodiments, the anti-inflammatory agent is a corticosteroid. Exemplary corticosteroids include, but are not limited to, cortisone (hydrocortisone, hydrocortisone sodium phosphate, hydrocortisone sodium succinate, Ala-Cort®, Hydrocort Acetate®, hydrocortone phosphate Lanacort®, Solu-Cortef®), decadron (dexamethasone, dexamethasone acetate, dexamethasone sodium phosphate, Dexasone®, Diodex®, Hexadrol®, Maxidex®), methylprednisolone (6-methylprednisolone, methylprednisolone acetate, methylprednisolone sodium succinate, Duralone®, Medralone®, Medrol®, M-Prednisol®, Solu-Medrol®), prednisolone (Delta-Cortef®, ORAPRED®, Pediapred®, Prezone®), and prednisone (Deltasone®, Liquid Pred®, Meticorten®, Orasone®), and bisphosphonates (e.g., pamidronate (Aredia®), and zoledronic acid (Zometac®).

In some embodiments, the anti-cancer therapy comprises an anti-hormonal agent. In some embodiments, the methods provided herein comprise administering to the individual an anti-hormonal agent, e.g., in combination with another anti-cancer therapy such as an immune checkpoint inhibitor. Anti-hormonal agents are agents that act to regulate or inhibit hormone action on tumors. Examples of anti-hormonal agents include anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGACE® megestrol acetate, AROMASIN® exemestane, formestanie, fadrozole, RIVISOR® vorozole, FEMARA® letrozole, and ARIMIDEX® (anastrozole); anti-androgens such as flutamide, nilutamide, bicalutamide, leuprolide, and goserelin; troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In some embodiments, the anti-cancer therapy comprises an antimetabolite chemotherapeutic agent. In some embodiments, the methods provided herein comprise administering to the individual an antimetabolite chemotherapeutic agent, e.g., in combination with another anti-cancer therapy such as an immune checkpoint inhibitor. Antimetabolite chemotherapeutic agents are agents that are structurally similar to a metabolite, but cannot be used by the body in a productive manner. Many antimetabolite chemotherapeutic agents interfere with the production of RNA or DNA. Examples of antimetabolite chemotherapeutic agents include gemcitabine (GEMZAR®), 5-fluorouracil (5-FU), capecitabine (XELODA™), 6-mercaptopurine, methotrexate, 6-thioguanine, pemetrexed, raltitrexed, arabinosylcytosine ARA-C cytarabine (CYTOSAR-U®), dacarbazine (DTIC-DOMED), azocytosine, deoxycytosine, pyridmidene, fludarabine (FLUDARA®), cladrabine, and 2-deoxy-D-glucose. In some embodiments, an antimetabolite chemotherapeutic agent is gemcitabine. Gemcitabine HCl is sold by Eli Lilly under the trademark GEMZAR®.

In some embodiments, the anti-cancer therapy comprises a platinum-based chemotherapeutic agent. In some embodiments, the methods provided herein comprise administering to the individual a platinum-based chemotherapeutic agent, e.g., in combination with another anti-cancer therapy such as an immune checkpoint inhibitor. Platinum-based chemotherapeutic agents are chemotherapeutic agents that comprise an organic compound containing platinum as an integral part of the molecule. In some embodiments, a chemotherapeutic agent is a platinum agent. In some such embodiments, the platinum agent is selected from cisplatin, carboplatin, oxaliplatin, nedaplatin, triplatin tetranitrate, phenanthriplatin, picoplatin, or satraplatin.

In some embodiments, the anti-cancer therapy comprises a cancer immunotherapy, such as a cancer vaccine, cell-based therapy, T cell receptor (TCR)-based therapy, adjuvant immunotherapy, cytokine immunotherapy, and oncolytic virus therapy. In some embodiments, the methods provided herein comprise administering to the individual a cancer immunotherapy, such as a cancer vaccine, cell-based therapy, T cell receptor (TCR)-based therapy, adjuvant immunotherapy, cytokine immunotherapy, and oncolytic virus therapy, e.g., in combination with another anti-cancer therapy such as an immune checkpoint inhibitor. In some embodiments, the cancer immunotherapy comprises a small molecule, nucleic acid, polypeptide, carbohydrate, toxin, cell-based agent, or cell-binding agent. Examples of cancer immunotherapies are described in greater detail herein but are not intended to be limiting. In some embodiments, the cancer immunotherapy activates one or more aspects of the immune system to attack a cell (e.g., a tumor cell) that expresses a neoantigen, e.g., a neoantigen expressed by a cancer of the disclosure. The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises a cancer vaccine. A range of cancer vaccines have been tested that employ different approaches to promoting an immune response against a cancer (see, e.g., Emens, Cancer vaccines: on the threshold of success, Expert Opin Emerg Drugs, vol. 13, no. 2, pp. 295-308 (2008) and US20190367613). Approaches have been designed to enhance the response of B cells, T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello et al., Development of a lipopeptide-based therapeutic vaccine to treat chronic HBV infection. L Induction of a primary cytotoxic T lymphocyte response in humans, J. Clin. Invest., vol. 95, pp. 341-349 (1995)), naked cDNA or peptides, encapsulated e.g., in poly(DL-lactide-co-glycolide) (“PLG”) microspheres (see, e.g., Eldridge et al., Biodegradable microspheres as a vaccine delivery system, Molec. Immunol., vol. 28, pp. 287-294 (1991): Alonso et al, Biodegradable microspheres as controlled-release tetanus toxoid delivery systems, Vaccine, vol. 12, pp. 299-306 (1994); Jones et al., Protection of mice from Bordetella pertussis respiratory infection using microencapsulated pertussis fimbriae, Vaccine, vol. 13, pp. 675-681 (1995)); peptide composition contained in immune stimulating complexes (ISCOMS) (e.g., Takahashi et al., Induction of CD8+ cytotoxic T cells by immunization with purified HIV-1 envelope protein in ISCOMs, Nature, vol. 344, pp. 873-875 (1990); Hu et al., The immunostimulating complex (ISCOM) is an efficient mucosal delivery system for respiratory syncytial virus (RSV) envelope antigens inducing high local and systemic antibody responses, Clin. Exp. Immunol., vol. 113, pp. 235-243 (1998)); or multiple antigen peptide systems (MAPs) (see e.g., Tam, Synthetic peptide vaccine design: synthesis and properties of a high-density multiple antigenic peptide system, Proc. Natl Acad. Sci. U.S.A., vol. 85, pp. 5409-5413 (1988); Tam, Recent advances in multiple antigen peptides, J. Immunol. Methods, vol. 196, pp. 17-32 (1996)). In some embodiments, a cancer vaccine is formulated as a peptide-based vaccine, or nucleic acid based vaccine in which the nucleic acid encodes the polypeptides. In some embodiments, a cancer vaccine is formulated as an antibody-based vaccine. In some embodiments, a cancer vaccine is formulated as a cell based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al., Next-generation peptide vaccines for advanced cancer, Cancer Sci, vol. 104, pp. 15-21 (2013)). In some embodiments, such cancer vaccines augment the anti-cancer response.

In some embodiments, the cancer vaccine comprises a polynucleotide that encodes a neoantigen, e.g., a neoantigen expressed by a cancer of the disclosure. In some embodiments, the cancer vaccine comprises DNA or RNA that encodes a neoantigen. In some embodiments, the cancer vaccine comprises a polynucleotide that encodes a neoantigen. In some embodiments, the cancer vaccine further comprises one or more additional antigens, neoantigens, or other sequences that promote antigen presentation and/or an immune response. In some embodiments, the polynucleotide is complexed with one or more additional agents, such as a liposome or lipoplex. In some embodiments, the polynucleotide(s) are taken up and translated by antigen presenting cells (APCs), which then present the neoantigen(s) via MHC class I on the APC cell surface.

In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543), prostate cancer (NCT01619813), head and neck squamous cell cancer (NCT01166542), pancreatic adenocarcinoma (NCT00998322), and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117), metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676), and peritoneal disease, colorectal cancer or ovarian cancer (NCT02963831); GL-ONC1 (GLV-lh68/GLV-lh153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260), fallopian tube cancer, ovarian cancer (NCT 02759588); or CGO070 (Cold Genesys), an adenovirus engineered to express GM-CSF in bladder cancer (NCT02365818); anti-gp100; STINGVAX; GVAX; DCVaxL; and DNX-2401. In some embodiments, the cancer vaccine is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into an individual (possibly with pro-inflammatory or other attractants such as GM-CSF), taken up by cells in vivo to make the specific antigens, which then provoke the desired immune response. In some embodiments, vectors may be used to deliver more than one tumor antigen at a time, to increase the immune response. In addition, recombinant virus, bacteria or yeast vectors can trigger their own immune responses, which may also enhance the overall immune response.

In some embodiments, the cancer vaccine comprises a DNA-based vaccine. In some embodiments, DNA-based vaccines can be employed to stimulate an anti-tumor response. The ability of directly injected DNA that encodes an antigenic protein, to elicit a protective immune response has been demonstrated in numerous experimental systems. Vaccination through directly injecting DNA that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al., Long-term anti-nucleoprotein cellular and humoral immunity is induced by intramuscular injection of plasmid DNA containing NP gene, DNA Cell Biol., vol. 12, pp. 771-776 (1993)). In some embodiments, plasmid (or other vector) DNA that includes a sequence encoding a protein operably linked to regulatory elements required for gene expression is administered to individuals (e.g. human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen.

In some embodiments, the cancer immunotherapy comprises a cell-based therapy. In some embodiments, the cancer immunotherapy comprises a T cell-based therapy. In some embodiments, the cancer immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) cancer cells. In some embodiments, the immune response results in inhibition of tumor and/or metastatic cell growth and/or proliferation, and in related embodiments, results in neoplastic cell death and/or resorption. The immune cells can be derived from a different organism/host (exogenous immune cells) or can be cells obtained from the subject organism (autologous immune cells). In some embodiments, the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g. chimeric). In some embodiments, a population of immune cells can be obtained from a subject in need of therapy or suffering from a disease associated with reduced immune cell activity. Thus, the cells will be autologous to the subject in need of therapy. In some embodiments, a population of immune cells can be obtained from a donor, such as a histocompatibility-matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible, in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy, such as autologous cells, e.g., tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. Several approaches for the isolation, derivation, engineering or modification, activation, and expansion of functional anti-tumor effector cells have been described in the last two decades and may be used according to any of the methods provided herein. In some embodiments, the T cells are derived from the blood, bone marrow, lymph, umbilical cord, or lymphoid organs. In some embodiments, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs).

In some embodiments, the T cell-based therapy comprises a chimeric antigen receptor (CAR)-T cell-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen.

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen.

In some embodiments, the T cell-based therapy comprises tumor-infiltrating lymphocytes (TILs). For example, TILs can be isolated from a tumor or cancer of the present disclosure, then isolated and expanded in vitro. Some or all of these TILs may specifically recognize an antigen expressed by the tumor or cancer of the present disclosure. In some embodiments, the TILs are exposed to one or more neoantigens, e.g., a neoantigen, in vitro after isolation. TILs are then administered to the patient (optionally in combination with one or more cytokines or other immune-stimulating substances).

In some embodiments, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells can be detected by specific surface markers, such as CD16, CD56, and CD8 in humans. NK cells do not express T-cell antigen receptors, the pan T marker CD3, or surface immunoglobulin B cell receptors. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art.

In some embodiments, the cell-based therapy comprises a dendritic cell (DC)-based therapy, e.g., a dendritic cell vaccine. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient. In some embodiments, immunization may be repeated multiple times if desired. Methods for harvesting, expanding, and administering dendritic cells are known in the art; see, e.g., WO2019178081. Dendritic cell vaccines (such as Sipuleucel-T, also known as APC8015 and PROVENGE®) are vaccines that involve administration of dendritic cells that act as APCs to present one or more cancer-specific antigens to the patient's immune system. In some embodiments, the dendritic cells are autologous or allogeneic to the recipient.

In some embodiments, the cancer immunotherapy comprises a TCR-based therapy. In some embodiments, the cancer immunotherapy comprises administration of one or more TCRs or TCR-based therapeutics that specifically bind an antigen expressed by a cancer of the present disclosure. In some embodiments, the TCR-based therapeutic may further include a moiety that binds an immune cell (e.g., a T cell), such as an antibody or antibody fragment that specifically binds a T cell surface protein or receptor (e.g., an anti-CD3 antibody or antibody fragment).

In some embodiments, the immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the immunotherapy comprises cytokine immunotherapy. Cytokine immunotherapy comprises the use of one or more cytokines that activate components of the immune system. Examples include, but are not limited to, aldesleukin (PROLEUKIN®; interleukin-2), interferon alfa-2a (ROFERON®-A), interferon alfa-2b (INTRON®-A), and peginterferon alfa-2b (PEGINTRON®).

In some embodiments, the immunotherapy comprises oncolytic virus therapy. Oncolytic virus therapy uses genetically modified viruses to replicate in and kill cancer cells, leading to the release of antigens that stimulate an immune response. In some embodiments, replication-competent oncolytic viruses expressing a tumor antigen comprise any naturally occurring (e.g., from a “field source”) or modified replication-competent oncolytic virus. In some embodiments, the oncolytic virus, in addition to expressing a tumor antigen, may be modified to increase selectivity of the virus for cancer cells. In some embodiments, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hcpadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. In some embodiments, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some embodiments, a replicative oncolytic vaccinia virus expressing a tumor antigen may be engineered to lack one or more functional genes in order to increase the cancer selectivity of the virus. In some embodiments, an oncolytic vaccinia virus is engineered to lack thymidine kinase (TK) activity. In some embodiments, the oncolytic vaccinia virus may be engineered to lack vaccinia virus growth factor (VGF). In some embodiments, an oncolytic vaccinia virus may be engineered to lack both VGF and TK activity. In some embodiments, an oncolytic vaccinia virus may be engineered to lack one or more genes involved in evading host interferon (IFN) response such as E3L, K3L, B18R, or B8R. In some embodiments, a replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen may be locally or systemically administered to a subject, e.g. via intratumoral, intraperitoneal, intravenous, intra-arterial, intramuscular, intradermal, intracranial, subcutaneous, or intranasal administration.

In some embodiments, the anti-cancer therapy comprises a nucleic acid molecule, such as a dsRNA, an siRNA, or an shRNA. In some embodiments, the methods provided herein comprise administering to the individual a nucleic acid molecule, such as a dsRNA, an siRNA, or an shRNA, e.g., in combination with another anti-cancer therapy. As is known in the art, dsRNAs having a duplex structure are effective at inducing RNA interference (RNAi). In some embodiments, the anti-cancer therapy comprises a small interfering RNA molecule (siRNA). dsRNAs and siRNAs can be used to silence gene expression in mammalian cells (e.g., human cells). In some embodiments, a dsRNA of the disclosure comprises any of between about 5 and about 10 base pairs, between about 10 and about 12 base pairs, between about 12 and about 15 base pairs, between about 15 and about 20 base pairs, between about 20 and 23 base pairs, between about 23 and about 25 base pairs, between about 25 and about 27 base pairs, or between about 27 and about 30 base pairs. As is known in the art, siRNAs are small dsRNAs that optionally include overhangs. In some embodiments, the duplex region of an siRNA is between about 18 and 25 nucleotides, e.g., any of 18, 19, 20, 21, 22, 23, 24, or 25 nucleotides. siRNAs may also include short hairpin RNAs (shRNAs), e.g., with approximately 29-base-pair stems and 2-nucleotide 3′ overhangs. Methods for designing, optimizing, producing, and using dsRNAs, siRNAs, or shRNAs, are known in the art.

### IX. NUCLEIC ACID EXTRACTION AND PROCESSING

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan et al., DNA, RNA, and Protein Extraction: The Past and The Present, J. Biomed. Biotech., vol. 2009, pp. 574398 (2009); the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin et al., Measurement of gene expression in archival paraffin-embedded tissues: development and performance of a 92-gene reverse transcriptase-polymerase chain reaction assay, Am J Pathol., vol. 164, no. 1, pp. 35-42 (2004); Masuda et al., Analysis of chemical modification of RNA from formalin-fixed samples and optimization of molecular biology applications for such samples, Nucleic Acids Res., vol. 27, no. 22, pp. 4436-4443 (1999); Specht et al., Quantitative gene expression analysis in microdissected archival formalin-fixed and paraffin-embedded tumor tissue, Am J Pathol., vol. 158, no. 2, pp. 419-429 (2001); the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### X. LIBRARY PREPARATION

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk et al., Library preparation methods for next-generation sequencing: tone down the bias, Exp. Cell Research, vol. 322, pp. 12-20 (2014), and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### XI. TARGETING GENE LOCI FOR ANALYSIS

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### XII. TARGET CAPTURE REAGENTS

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### XIII. HYBRIDIZATION CONDITIONS

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert et al., Direct selection of human genomic loci by microarray hybridization, Nat. Methods, vol. 4, no. 11, pp. 903-905 (2007); Hodges et al., Genome-wide in situ exon capture for selective resequencing, Nat. Genet., vol. 39, no. 12, pp. 1522-1527 (2007); and Okou et al., Microarray-based genomic selection for high-throughput resequencing, Nat. Methods, vol. 4, no. 11, pp. 907-909 (2007), the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### XIV. SEQUENCING METHODS

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker et al., Sequencing technologies—the next generation, Nature Biotechnology Reviews, vol. 11, pp. 31-46 (2010), which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLiD, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### XV. ALIGNMENT

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell et al., How to map billions of short reads onto genomes, Nature Biotech., vol 27, pp. 455-457 (2009). Examples of de novo sequence assemblies are described in, e.g., Warren et al., Assembling millions of short DNA sequences using SSAKE, Bioinformatics, vol. 23, pp. 500-501 (2007); Butler et al., ALLPATHS: de novo assembly of whole-genome shotgun microreads, Genome Res., vol. 18, pp. 810-820 (2008); and Zerbino et al., Velvet: algorithms for de novo short read assembly using de Bruijn graphs, Genome Res., vol. 18, pp. 821-829 (2008). Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li et al., Fast and Accurate Short Read Alignment with Burrows-Wheeler Transform, Bioinformatics, vol. 25, pp. 1754-1760 (2009); Li et al., Fast and Accurate Long-Read Alignment with Burrows-Wheeler Transform, Bioinformatics, epub. PMID: 20080505 (2010)), the Smith-Waterman algorithm (see, e.g., Smith et al., Identification of Common Molecular Subsequences, J. Molecular Biology, vol. 147, no. 1, pp. 195-197 (1981)), the Striped Smith-Waterman algorithm (see, e.g., Farrar et al., Striped Smith-Waterman Speeds Database Searches Six Times Over Other SIMD Implementations, Bioinformatics, vol. 23, no. 2, pp. 156-161 (2007)), the Needleman-Wunsch algorithm (Needleman et al., A General Method Applicable to the Search for Similarities in the Amino Acid Sequence of Two Proteins, J. Molecular Biology, vol. 48, no. 3, pp. 443-53 (1970)), or any combination thereof.

In some instances, the methods and systems disclosed herein may also comprise the use of a sequence assembly algorithm, e.g., the Arachne sequence assembly algorithm (see, e.g., Batzoglou et al., ARACHNE: A Whole-Genome Shotgun Assembler, Genome Res., vol. 12, pp. 177-189 (2002)).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### XVI. MUTATION CALLING

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li et al., Fast and accurate long-read alignment with Burrows-Wheeler transform, Bioinformatics, vol. 26, no. 5, pp. 589-95 (2010). The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning et al., Simultaneous genotype calling and haplotype phasing improves genotype accuracy and reduces false-positive associations for genome-wide association studies, Am. J. Hum. Genet., vol. 85, no. 6, pp. 847-861 (2009). Examples of low-coverage SNP calling methods are described in, e.g., Li et al., Genotype imputation, Annu. Rev. Genomics Hum. Genet., vol. 10, pp. 387-406 (2009).

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ~1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna et al., The Genome Analysis Toolkit: a MapReduce framework for analyzing next-generation DNA sequencing data, Genome Res., vol. 20, no. 9, pp. 1297-1303 (2010); Ye et al., Pindel: a pattern growth approach to detect break points of large deletions and medium sized insertions from paired-end short reads, Bioinformatics, vol. 25, no. 21, pp. 2865-2871 (2009); Lunter et al., Stampy: a statistical algorithm for sensitive and fast mapping of Illumina sequence reads, Genome Res., vol. 21, no. 6, pp. 936-939 (2011); and Li et al., The Sequence Alignment/Map format and SAMtools, Bioinformatics, vol. 25, no. 16, pp. 2078-2079 (2009).

Methods for generating indel calls and individual-level genotype likelihoods include, e.g., the Dindel algorithm (Albers et al., Dindel: accurate indel calls from short-read data, Genome Res., vol. 21, no. 6, pp. 961-973 (2011)). For example, the Bayesian EM algorithm can be used to analyze the reads, make initial indel calls, and generate genotype likelihoods for each candidate indel, followed by imputation of genotypes using, e.g., QCALL (Le et al., SNP detection and genotyping from low-coverage sequencing data on multiple diploid samples, Genome Res., vol. 21, no. 6, pp. 952-960 (2011)). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., Goya et al., SNVMix: predicting single nucleotide variants from next-generation sequencing of tumors, Bioinformatics., vol. 26, no. 6, pp. 730-736 (2010)) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of mutation calling methods is provided in, e.g., International Patent Application Publication No. WO 2020/236941, the entire content of which is incorporated herein by reference.

### XVII. TYPES OF ALTERATIONS

Various types of alterations (e.g., somatic alterations) can be evaluated and used for the analysis of mutation load, in a method as described herein.

### A. Somatic Alterations

In certain embodiments, the alteration evaluated in accordance with a method described herein is an alteration (e.g., a somatic alteration).

In certain embodiments, the alteration (e.g., somatic alteration) is a coding short variant, e.g., a base substitution or an indel (insertion or deletion). In certain embodiments, the alteration (e.g., somatic alteration) is a point mutation. In other embodiments, the alteration (e.g., somatic alteration) is other than a rearrangement, e.g., other than a translocation. In certain embodiments, the alteration (e.g., somatic alteration) is a splice variant.

In certain embodiments, the alteration (e.g., somatic alteration) is a silent mutation, e.g., a synonymous alteration. In other embodiments, the alteration (e.g., somatic alteration) is a non-synonymous single nucleotide variant (SNV). In other embodiments, the alteration (e.g., somatic alteration) is a passenger mutation, e.g., an alteration that has no detectable effect on the fitness of a clone of cells. In certain embodiments, the alteration (e.g., somatic alteration) is a variant of unknown significance (VUS), e.g., an alteration, the pathogenicity of which can neither be confirmed nor ruled out. In certain embodiments, the alteration (e.g., somatic alteration) has not been identified as being associated with a cancer phenotype.

In certain embodiments, the alteration (e.g., somatic alteration) is not associated with, or is not known to be associated with, an effect on cell division, growth or survival. In other embodiments, the alteration (e.g., somatic alteration) is associated with an effect on cell division, growth or survival.

In certain embodiments, an increased level of a somatic alteration is an increased level of one or more classes or types of a somatic alteration (e.g., a rearrangement, a point mutation, an indel, or any combination thereof). In certain embodiments, an increased level of a somatic alteration is an increased level of one class or type of a somatic alteration (e.g., a rearrangement only, a point mutation only, or an indel only). In certain embodiments, an increased level of a somatic alteration is an increased level of a somatic alteration at a preselected position (e.g., an alteration described herein). In certain embodiments, an increased level of a somatic alteration is an increased level of a preselected somatic alteration (e.g., an alteration described herein).

### B. Functional Alterations

In certain embodiments, the number of an alteration (e.g., a somatic alteration) excludes a functional alteration in a subgenomic interval.

In some embodiments, the functional alteration is an alteration that, compared with a reference sequence, e.g., a wild-type or unmutated sequence, has an effect on cell division, growth or survival, e.g., promotes cell division, growth or survival. In certain embodiments, the functional alteration is identified as such by inclusion in a database of functional alterations, e.g., the COSMIC database (cancer.sanger.ac.uk/cosmic; Forbes et al., COSMIC: exploring the world's knowledge of somatic mutations in human cancer, Nucl. Acids Res., vol. 43, pp. D805-D811 (2015)). In other embodiments, the functional alteration is an alteration with known functional status, e.g., occurring as a known somatic alteration in the COSMIC database. In certain embodiments, the functional alteration is an alteration with a likely functional status, e.g., a truncation in a tumor suppressor gene. In certain embodiments, the functional alteration is a driver mutation, e.g., an alteration that gives a selective advantage to a clone in its microenvironment, e.g., by increasing cell survival or reproduction. In other embodiments, the functional alteration is an alteration capable of causing clonal expansions. In certain embodiments, the functional alteration is an alteration capable of causing one, two, three, four, five, or all of the following: (a) self-sufficiency in a growth signal; (b) decreased, e.g., insensitivity, to an antigrowth signal; (c) decreased apoptosis; (d) increased replicative potential; (e) sustained angiogenesis; or (f) tissue invasion or metastasis.

In certain embodiments, the functional alteration is not a passenger mutation, e.g., is not an alteration that has no detectable effect on the fitness of a clone of cells. In certain embodiments, the functional alteration is not a variant of unknown significance (VUS), e.g., is not an alteration, the pathogenicity of which can neither be confirmed nor ruled out.

In certain embodiments, a plurality (e.g., about 10%, 20%, 30%, 40%, 50%, 60%, 70%, 80%, 90%, or more) of functional alterations in a preselected tumor gene in the predetermined set of genes are excluded. In certain embodiments, all functional alterations in a preselected gene (e.g., tumor gene) in the predetermined set of genes are excluded. In certain embodiments, a plurality of functional alterations in a plurality of preselected genes (e.g., tumor genes) in the predetermined set of genes are excluded. In certain embodiments, all functional alterations in all genes (e.g., tumor genes) in the predetermined set of genes are excluded.

### C. Germline Mutations

In certain embodiments, the number of an alteration excludes a germline mutation in a subgenomic interval. In certain embodiments, the somatic alteration is not identical or similar to, e.g., is distinguishable from, a germline mutation.

In certain embodiments, the germline alteration is a single nucleotide polymorphism (SNP), a base substitution, an indel (e.g., an insertion or a deletion), or a silent mutation (e.g., synonymous mutation).

In certain embodiments, the germline alteration is excluded by use of a method that does not use a comparison with a matched normal sequence. In other embodiments, the germline alteration is excluded by a method comprising the use of an SGZ algorithm. In certain embodiments, the germline alteration is identified as such by inclusion in a database of germline alterations, e.g., the dbSNP database (www.ncbi.nlm.nih.gov/SNP/index.html; Sherry et al., dbSNP: the NCBI database of genetic variation, Nucleic Acids Res., vol. 29, no. 1, pp. 308-311 (2001)). In other embodiments, the germline alteration is identified as such by inclusion in two or more counts of the ExAC database (exac.broadinstitute.org; Exome Aggregation Consortium et al. “Analysis of protein-coding genetic variation in 60,706 humans,” bioRxiv preprint. Oct. 30, 2015). In some embodiments, the germline alteration is identified as such by inclusion in the 1000 Genome Project database (www.1000genomes.org; McVean et al., An integrated map of genetic variation from 1,092 human genomes, Nature, vol. 491, pp. 56-65 (2012)). In some embodiments, the germline alteration is identified as such by inclusion in the ESP database (Exome Variant Server, NHLBI GO Exome Sequencing Project (ESP), Seattle, WA (evs.gs.washington.edu/EVS/).

### XVIII. SYSTEMS

Also disclosed herein are systems designed to implement any of the disclosed methods for determining clonal tumor mutational burden (cTMB) in a sample from a subject. The systems may comprise, e.g., one or more processors, and a memory unit communicatively coupled to the one or more processors and configured to store instructions that, when executed by the one or more processors, cause the system to receive a plurality of sequence read data that represent a set of nucleic acid molecules obtained from a sample for a subject. The systems may further comprise, e.g., one or more processors, and a memory unit communicatively coupled to the one or more processors and configured to store instructions that, when executed by the one or more processors, cause the system to identify one or more short variants from the sequence read data. The systems may comprise, e.g., one or more processors, and a memory unit communicatively coupled to the one or more processors and configured to store instructions that, when executed by the one or more processors, cause the system to filter the one or more short variants to select one or more clonal tumor mutational burden (cTMB)-qualified short variants. In some embodiments, the filtering comprises excluding short variants identified as subclonal based on a tumor amount measure for a respective short variant. For example, the tumor amount measure for a respective short variant may comprise a cancer cell fraction (CCF) of the variant or a variant allele frequency (VAF). The systems may further comprise, e.g., one or more processors, and a memory unit communicatively coupled to the one or more processors and configured to store instructions that, when executed by the one or more processors, cause the system to determine a cTMB value for the sample based on the number of cTMB-qualified short variants.

The systems may comprise, e.g., one or more processors, and a memory unit communicatively coupled to the one or more processors and configured to store instructions that, when executed by the one or more processors, cause the system to receive genomic data for a subject. In some embodiments, the genomic data indicates a presence or absence of one or more short variants in a sample derived from the subject. The systems may comprise, e.g., one or more processors, and a memory unit communicatively coupled to the one or more processors and configured to store instructions that, when executed by the one or more processors, cause the system to filter the one or more short variants to select one or more clonal tumor mutational burden (cTMB)-qualified short variants. In some embodiments, the filtering comprises excluding short variants identified as subclonal based on a tumor amount measure for a respective short variant. For example, the tumor amount measure for a respective short variant may comprise a cancer cell fraction (CCF) of the variant or a variant allele frequency (VAF). The systems may comprise, e.g., one or more processors, and a memory unit communicatively coupled to the one or more processors and configured to store instructions that, when executed by the one or more processors, cause the system to determine a cTMB value for the sample based on the number of cTMB-qualified short variants.

In some embodiments, the instructions further cause the system to select a treatment for the subject, wherein the subject has a cancer, based on the cTMB for the sample. In some embodiments, if the cTMB determined for the sample is at or above a threshold cTMB, the subject is identified as one who may benefit from treatment with an immune-oncology (IO) therapy. In some embodiments, if the cTMB determined for the sample is below a threshold cTMB, the subject is identified as one who may not benefit from treatment with an IO therapy.

In some embodiments, the instructions further cause the system to generate a report comprising one or more treatment options identified for the subject based at least in part on the cTMB determined for the sample. In some embodiments, if the cTMB is at or above a threshold cTMB, the subject is identified as one who may benefit from treatment with an immune-oncology (IO) therapy. In some embodiments, if the cTMB is below a threshold cTMB, the subject is identified as one who may not benefit from treatment with an IO therapy.

In some embodiments, the instructions further cause the system to predict survival of the subject, wherein the subject has cancer. In some embodiments, if the cTMB determined for the sample is at or above a threshold cTMB, the subject is predicted to have longer survival when treated with an immune-oncology (IO) therapy, as compared to a subject determined to have a cTMB below the threshold cTMB.

In some embodiments, the cTMB is determined by one or more of (a) counting a number of the one or more clonal short variants in the sample, and (b) normalizing a size of a bait molecule.

In some embodiments, the tumor amount measure of the system comprises a cancer cell fraction (CCF) or a variant allele frequency (VAF). In some embodiments, the tumor amount measure comprises a CCF. In some embodiments, the CCF is a value between 0 and 1, wherein the CCF value represents a percentage of cancer cells containing the one or more variant in the cancer cell population. The CCF represents a simple but effective characteristic of a tumor sample. For example, a CCF equal to 0.50 represents 50% of the cancer cell population contains the one or more short variant. In some embodiments, the tumor amount measure comprises a CCF that is at least 0.30, at least 0.35, at least 0.40, at least 0.45, at least 0.50, at least 0.55, at least 0.60, at least 0.65, at least 0.70, at least 0.75, at least 0.80, or more. In some embodiments, the tumor amount measure comprises a CCF that is between 0.30 and 0.80, 0.35 and 0.75, 0.40 and 0.60, or 0.45 and 0.55. In some embodiments, the tumor amount measure comprises a CCF that is at least 0.40. In some embodiments, the tumor amount measure comprises a CCF that is at least 0.45. In some embodiments, the tumor amount measure comprises a CCF that is at least 0.50. In some embodiments, the tumor amount measure comprises a CCF that is at least 0.55. In some embodiments, the tumor amount measure having a CCF is used by the system to determine a clonal tumor mutational burden (cTMB) for the sample associated with the tumor amount measure.

In some embodiments, the tumor amount measure of the system comprises a cancer cell fraction (CCF) or a variant allele frequency (VAF). In some embodiments, the tumor amount measure comprises a VAF. In some embodiments, the VAF is less than 10%, less than 9%, less than 7%, less than 6%, less than 5%, less than 4%, less than 3%, less than 2%, less than 1%, less than 0.5%, or less than 0.1%. In some embodiments, the VAF is no more than 10%, no more than 9%, no more than 7%, no more than 6%, no more than 5%, no more than 4%, no more than 3%, no more than 2%, no more than 1%, no more than 0.5%, or no more than 0.1%. In some embodiments, the VAF is less than 10%. In some embodiments, the VAF is no more than 10%. In some embodiments, the VAF is less than 5%. In some embodiments, the VAF is no more than 5%. In some embodiments, the tumor amount measure having a VAF is used by the system to determine a clonal tumor mutational burden (cTMB) for the sample associated with the tumor amount measure.

In some embodiments, the system the filter step comprises excluding short variants identified as having an allele frequency below a predetermined threshold. In some embodiments, the predetermined threshold for the variant allele frequency (VAF) is less than 10%, less than 9%, less than 7%, less than 6%, less than 5%, less than 4%, less than 3%, less than 2%, less than 1%, less than 0.5%, or less than 0.1%. In some embodiments, the predetermined threshold for the VAF is no more than 10%, no more than 9%, no more than 7%, no more than 6%, no more than 5%, no more than 4%, no more than 3%, no more than 2%, no more than 1%, no more than 0.5%, or no more than 0.1%. In some embodiments, the predetermined threshold for the VAF is less than 10%. In some embodiments, the predetermined threshold for the VAF is no more than 10%. In some embodiments, the predetermined threshold for the VAF is less than 5%. In some embodiments, the predetermined threshold for the VAF is no more than 5%. In some embodiments, the predetermined threshold for the VAF is used to determine if a variant is subclonal. In some embodiments, the filtering comprises excluding short variants identified as subclonal based on a tumor amount measure for a respective short variant. In some embodiments, the subclonal variant is a variant having a VAF below a certain threshold. In some embodiments, the tumor amount measure having a predetermined threshold for VAF is used by the system to determine a clonal tumor mutational burden (cTMB) for the sample associated with the tumor amount measure.

In some embodiments, the system comprising the filter step comprises excluding short variants identified as a germline variant. In some embodiments, the tumor amount measure excluding germline variants is used by the system to determine a clonal tumor mutational burden (cTMB) for the sample associated with the tumor amount measure.

In some embodiments, the system comprising filter step comprises excluding short variants identified as having a cancer cell fraction (CCF) at or below a predetermined CCF threshold.

In some embodiment, one or more short variants are not included for analysis if the one or more short variants are below a predetermined cancer cell fraction (CCF) threshold. In some embodiment, one or more short variants are included for analysis if the one or more short variants are at or above a predetermined CCF threshold. In some embodiments, the CCF is a value between 0 and 1, wherein the CCF value represents a percentage of cancer cells containing the one or more variant in the cancer cell population. For example, a CCF equal to 0.50 represents 50% of the cancer cell population contains the one or more short variant. In some embodiments, the predetermined CCF threshold for filtering the one or more short variants is at least 0.30, at least 0.35, at least 0.40, at least 0.45, at least 0.50, at least 0.55, at least 0.60, at least 0.65, at least 0.70, at least 0.75, at least 0.80, or more. In some embodiments, the predetermined CCF threshold for filtering the one or more short variants is between 0.30 and 0.80, 0.35 and 0.75, 0.40 and 0.60, or 0.45 and 0.55. In some embodiments, the predetermined CCF threshold for filtering the one or more short variants is at least 0.40. In some embodiments, the predetermined CCF threshold for filtering the one or more short variants is at least 0.45. In some embodiments, the predetermined CCF threshold for filtering the one or more short variants is at least 0.50. In some embodiments, the predetermined CCF threshold for filtering the one or more short variants is at least 0.55. In some embodiments, the tumor amount measure having a predetermined CCF threshold is used by the system to determine a clonal tumor mutational burden (cTMB) for the sample associated with the tumor amount measure.

In some embodiments, the system comprises a clonal tumor mutational burden (cTMB) value, where the cTMB value is expressed as a function of the number of cTMB-qualified short variants per megabase (mutations/Mb) in a set of subgenomic intervals from the sample. In some embodiments, the set of subgenomic intervals from the sample are between about 100 kb to about 10 Mb. In some embodiments, the set of subgenomic intervals from the sample are between about 100 kb to about 10 Mb. In some embodiments, the set of subgenomic intervals from the sample are between about 100 kb to about 10 Mb, 200 kb to about 9 Mb, 300 kb to about 8 Mb, 400 kb to about 7 Mb, 500 kb to about 6 Mb, 600 kb to about 5 Mb, 700 kb to about 4 Mb, 800 kb to about 3 Mb, or 900 kb to about 2 Mb. In some embodiments, the set of subgenomic intervals from the sample are at least 0.1 Mb, 0.2 Mb, 0.3 Mb, 0.4 Mb, 0.5 Mb, 0.6 Mb, 0.7 Mb, 0.8 Mb, 0.9 Mb, 1 Mb, 2 Mb, 3 Mb, 4 Mb, 5 Mb, 6 Mb, 7 Mb, 8 Mb, 9 Mb, or more. In some embodiments, the set of subgenomic intervals from the sample are no more than 0.2 Mb, 0.3 Mb, 0.4 Mb, 0.5 Mb, 0.6 Mb, 0.7 Mb, 0.8 Mb, 0.9 Mb, 1 Mb, 2 Mb, 3 Mb, 4 Mb, 5 Mb, 6 Mb, 7 Mb, 8 Mb, 9 Mb, or 10 Mb. In some embodiments, the set of subgenomic intervals from the sample are between about 0.8 Mb to about 1.1 Mb. In some embodiments, the set of subgenomic intervals from the sample are between 0.8 Mb to 1.1 Mb.

In some embodiments, the system comprises a clonal tumor mutational burden (cTMB) value, where the cTMB value is expressed as a function of the number of cTMB-qualified short variants per megabase (mutations/Mb) in a set of subgenomic intervals from the sample. In some embodiments, the cTMB value from the sample is between about 0.1 mutations/Mb to about 100 mutations/Mb. In some embodiments, the cTMB value from the sample is between about 0.1 mutations/Mb to about 100 mutations/Mb, about 0.2 mutations/Mb to about 90 mutations/Mb, about 0.3 mutations/Mb to about 80 mutations/Mb, about 0.4 mutations/Mb to about 70 mutations/Mb, about 0.5 mutations/Mb to about 60 mutations/Mb, about 1 mutations/Mb to about 50 mutations/Mb, about 2 mutations/Mb to about 40 mutations/Mb, about 5 mutations/Mb to about 30 mutations/Mb, or about 10 mutations/Mb to about 20 mutations/Mb. In some embodiments, the cTMB value from the sample is at least 0.1 mutations/Mb, 0.2 mutations/Mb, 0.3 mutations/Mb, 0.4 mutations/Mb, 0.5 mutations/Mb, 1 mutations/Mb, 2 mutations/Mb, 5 mutations/Mb, 10 mutations/Mb, 15 mutations/Mb, 20 mutations/Mb, 30 mutations/Mb, 40 mutations/Mb, 50 mutations/Mb, 60 mutations/Mb, 70 mutations/Mb, 80 mutations/Mb, 90 mutations/Mb, or more. In some embodiments, the cTMB value from the sample is no more than 0.2 mutations/Mb, 0.3 mutations/Mb, 0.4 mutations/Mb, 0.5 mutations/Mb, 1 mutations/Mb, 2 mutations/Mb, 5 mutations/Mb, 10 mutations/Mb, 15 mutations/Mb, 20 mutations/Mb, 30 mutations/Mb, 40 mutations/Mb, 50 mutations/Mb, 60 mutations/Mb, 70 mutations/Mb, 80 mutations/Mb, 90 mutations/Mb, or 100 mutations/Mb. In some embodiments, the cTMB value from the sample is between about 5 mutations/Mb to about 20 mutations/Mb. In some embodiments, the cTMB value from the sample is between 5 mutations/Mb to 20 mutations/Mb.

In some embodiments, the genomic data for the subject is based on a targeted exome sequencing panel.

In some embodiments, genomic data for the subject used by the system is derived from a single sample. Examples of a sample include, but are not limited to, a tumor sample, a tissue sample, a biopsy sample, a blood sample, a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample. In some embodiments, the genomic data for the subject is derived from a single biopsy sample. In some embodiments, the genomic data for the subject is derived from only one biopsy sample. In some embodiments, the genomic data for the subject is derived from circulating tumor DNA in a liquid biopsy sample. In some instances, the liquid biopsy sample may comprise, for example, whole blood, blood plasma, blood serum, urine, stool, sputum, saliva, or cerebrospinal fluid. In some embodiments, the liquid biopsy sample may comprise circulating tumor cells (CTCs). In some embodiments, the liquid biopsy sample and may comprise cell-free DNA (cfDNA), circulating tumor DNA (ctDNA), or any combination thereof.

In some embodiments, the genomic data for the subject used by the system is derived from single cell sequencing. In some embodiments, the one or more short variants include noncoding and synonymous short variants.

In some embodiments, the system further comprises obtaining the sample from the subject. In some embodiments, the subject is a human. In some embodiments, the subject has a disease, for example, cancer. In some embodiments, the subject has previously been treated with an anti-cancer therapy. In some embodiments, the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof. In some embodiments, the subject has not previously been treated with an anti-cancer therapy. In some embodiments, the sample comprises a tissue biopsy sample, a liquid biopsy sample, or a normal control. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof.

In some embodiments, the set of nucleic acid molecules used by the system is a plurality of nucleic acid molecules. In some embodiments, the plurality of nucleic acid molecules comprises a mixture of tumor nucleic acid molecules and non-tumor nucleic acid molecules. In some embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In some embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In some embodiments, the genomic data used by the system is obtained from sequencing the sample. In some embodiments, the sequencing comprises use of a massively parallel sequencing (MPS) technique, whole genome sequencing (WGS), whole exome sequencing, targeted sequencing, direct sequencing, or Sanger sequencing technique. In some embodiments, the sequencing comprises massively parallel sequencing, and the massively parallel sequencing technique comprises next generation sequencing (NGS).

In some embodiments, the system comprising sequencing comprises providing a plurality of nucleic acid molecules obtained from the sample from the subject. In some embodiments, the sequencing further comprises ligating one or more adapters onto one or more nucleic acid molecules from the plurality of nucleic acid molecules. In some embodiments, the sequencing further comprises amplifying the one or more ligated nucleic acid molecules from the plurality of nucleic acid molecules. In some embodiments, the sequencing further comprises capturing amplified nucleic acid molecules from the amplified nucleic acid molecules. In some embodiments, the sequencing further comprises sequencing, by a sequencer, the captured nucleic acid molecules to obtain a plurality of sequence reads that represent the set of nucleic acid molecules in the sample.

In some embodiments, the one or more adapters of the sequencing used by the system comprise amplification primers, flow cell adapter sequences, unique molecular identifier sequence, substrate adapter sequences, sample index sequences, or combination thereof. In some embodiments, the captured nucleic acid molecules are captured from the amplified nucleic acid molecules by hybridization to one or more bait molecules. In some embodiments, the one or more bait molecules comprise one or more nucleic acid molecules, each comprising a region that is complementary to a region of a captured nucleic acid molecule. In some embodiments, amplifying nucleic acid molecules comprises performing a polymerase chain reaction (PCR) amplification technique, a non-PCR amplification technique, or an isothermal amplification technique.

In some embodiments, the plurality of sequence reads used by the system overlap one or more gene loci within a subgenomic interval in the sample. In some embodiments, the one or more gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some embodiments, the disclosed systems may be used to identify variants in the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFIl, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene locus, or any combination thereof.

In some instances, the disclosed systems may be used to identify variants in the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-10, IL-6, IL-6R, JAK1, JAK2, JAK3, KIT, KRAS, MEK, MET, MSI-H, mTOR, PARP, PD-1, PDGFR, PDGFRa, PDGFRβ, PD-L1, PI3Kδ, PIGF, PTCH, RAF, RANKL, RET, ROS1, SLAMF7, VEGF, VEGFA, or VEGFB gene locus, or any combination thereof.

In some embodiments, the plurality of sequence reads used by the system overlap one or more gene loci within a subgenomic interval in the sample. In some embodiments, the one or more gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci. In some embodiments, the one or more short variants comprise ALOX12B T402T, ATRX K2225del, CTNNA1 D362N, FLT3 S102S, JAK2 I289V, KEAP1 K97N, KRAS Q61H, MSH3 R573fs*4, PDK1 T306M, STAG2 C527F, STK11 649_650delCC, TSC2 A68S, or any combination thereof. In some embodiments, the one or more cTMB-qualified short variants comprise CTNNA1 D362N, FLT3 S102S, STAG2 C527F, TSC2 A68S, or any combination thereof.

In some embodiments, the one or more cTMB-qualified short variants used by the system comprise CTNNA1 D362N. Catenin alpha-1 (CTNNA1; UniProt #P35221) is a protein that associates with the cytoplasmic domain of a variety of cadherins to form a complex which is linked to the actin filament network. CTNNA1 may play a crucial role in cell differentiation. In some embodiments, the one or more cTMB-qualified short variants used by the system comprise FLT3 S102S. Receptor-type tyrosine-protein kinase (FLT3; UniProt #P36888) is a tyrosine-protein kinase that helps to regulate differentiation, proliferation and survival of hematopoietic progenitor cells and dendritic cells. In some embodiments, the one or more cTMB-qualified short variants used by the system comprise STAG2 C527F. Cohesin subunit SA-2 (STAG2; UniProt #Q8N3U4) is a component of cohesin complex, which is required for the cohesion of sister chromatids after DNA replication. In some embodiments, the one or more cTMB-qualified short variants used by the system comprise TSC2 A68S. Tuberin (TSC2; UniProt #P49815), in complex with TSC1, functions as a tumor suppressor that regulate mTORC1 signaling. In some embodiments, the one or more cTMB-qualified short variants used by the system comprise CTNNA1 D362N, FLT3 S102S, STAG2 C527F, TSC2 A68S, or any combination thereof.

In some embodiments, the system further comprises generating, by the one or more processors, a report comprising a cTMB for the subject. In some embodiments, the system further comprising transmitting the report to a healthcare provider. In some embodiments, the report is transmitted via a computer network or a peer-to-peer connection.

In some embodiments, the threshold cTMB value used by the system is at least about 1 to 100 mutations/Mb, about 2 to 90 mutations/Mb, 3 to 80 mutations/Mb, 5 to 70 mutations/Mb, 10 to 60 mutations/Mb, 20 to 50 mutations/Mb, or 30 to 40 mutations/Mb. In some embodiments, the threshold cTMB value used by the system is less than less than 2 mutations/Mb, 3 mutations/Mb, less than 5 mutations/Mb, 10 mutations/Mb, 20 mutations/Mb, 30 mutations/Mb, 50 mutations/Mb, 75 mutations/Mb, or 100 mutations/Mb. In some embodiments, the threshold cTMB value used by the system is at least about 1 mutations/Mb, at least about 2 mutations/Mb, at least about 3 mutations/Mb, at least about 5 mutations/Mb, at least about 10 mutations/Mb, at least about 20 mutations/Mb, at least about 30 mutations/Mb, at least about 40 mutations/Mb, at least about 50 mutations/Mb, at least about 60 mutations/Mb, at least about 70 mutations/Mb, at least about 80 mutations/Mb, at least about 90 mutations/Mb, or at least about 100 mutations/Mb. In some embodiments, the threshold cTMB value used by the system is at least about 5 mutations/Mb, at least about 6 mutations/Mb, at least 7 mutations/Mb, at least about 8 mutations/Mb, at least about 9 mutations/Mb, at least about 10 mutations/Mb, at least about 11 mutations/Mb, at least about 12 mutations/Mb, at least about 13 mutations/Mb, at least about 14 mutations/Mb, at least about 15 mutations/Mb, at least about 16 mutations/Mb, at least about 17 mutations/Mb, at least about 18 mutations/Mb, at least about 19 mutations/Mb, at least about 20 mutations/Mb, at least about 21 mutations/Mb, at least about 22 mutations/Mb, at least about 23 mutations/Mb, at least about 24 mutations/Mb, or at least about 25 mutations/Mb.

In some embodiments, the system comprises a threshold cTMB value that is at least about 4 to 100 mutations/Mb, about 4 to 30 mutations/Mb, 8 to 100 mutations/Mb, 8 to 30 mutations/Mb, 10 to 20 mutations/Mb, less than 4 mutations/Mb, or less than 8 mutations/Mb. In some embodiments, the system comprises a threshold cTMB value is at least about 5 mutations/Mb, at least about 10 mutations/Mb, at least about 12 mutations/Mb, at least about 16 mutations/Mb, at least about 20 mutations/Mb, or at least about 30 mutations/Mb. In some embodiments, the system comprises a threshold cTMB value is at least about 7.5 mutations/Mb, at least about 8.5 mutations/Mb, at least about 9.5 mutations/Mb, at least about 11 mutations/Mb, at least about 13.5 mutations/Mb, at least about 16.5 mutations/Mb, at least about 18.5 mutations/Mb, at least about 19 mutations/Mb, or at least about 25 mutations/Mb.

In some embodiments, the immuno-oncology (IO) therapy identified by the system comprises a single IO agent or multiple IO agents. In some embodiments, the IO therapy identified by the system comprises an immune checkpoint inhibitor.

In some embodiments, the immune checkpoint inhibitor identified by the system comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a cellular therapy, a treatment for cancer being tested in a clinical trial, an immunotherapy, or any combination thereof.

In some embodiments, the immuno-oncology (IO) therapy selected by the system for treatment of a subject comprises a single IO agent or multiple IO agents. In some embodiments, the IO therapy selected by the system for treatment of a subject comprises an immune checkpoint inhibitor.

In some embodiments, the immune checkpoint inhibitor selected by the system for treatment of a subject comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a cellular therapy, a treatment for cancer being tested in a clinical trial, an immunotherapy, or any combination thereof.

In some embodiments, the immuno-oncology (IO) therapy determined to be beneficial of a subject in need comprises a single IO agent or multiple IO agents. In some embodiments, the IO therapy determined to be beneficial of a subject in need comprises an immune checkpoint inhibitor.

In some embodiments, the immune checkpoint inhibitor determined to be beneficial of a subject in need comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a cellular therapy, a treatment for cancer being tested in a clinical trial, an immunotherapy, or any combination thereof.

In some embodiments, the immuno-oncology (IO) therapy identified and/or selected by the system comprises an immune checkpoint inhibitor, for example, an immune checkpoint inhibitor targeting PD-1, PD-L1, CTLA-4, or a combination thereof. In some embodiments, the immune checkpoint inhibitor is a PD-1 inhibitor. In some embodiments, the immune checkpoint inhibitor comprises one or more of nivolumab, pembrolizumab, cemiplimab, or dostarlimab. In some embodiments, the immune checkpoint inhibitor is a PD-L1-inhibitor. In some embodiments, the immune checkpoint inhibitor comprises one or more of atezolizumab, avelumab, or durvalumab. In some embodiments, the immune checkpoint inhibitor is a CTLA-4 inhibitor. In some embodiments, the CTLA-4 inhibitor comprises ipilimumab.

In some embodiments, the nucleic acid identified and/or selected by the system comprises a double-stranded RNA (dsRNA), a small interfering RNA (siRNA), or a small hairpin RNA (shRNA).

In some embodiments, the cellular therapy identified and/or selected by the system is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In some embodiments, the system further comprises treating the subject with the IO therapy. In some embodiments, the system further comprises treating the subject with an additional anti-cancer therapy. In some embodiments, the additional anti-cancer therapy comprises one or more of a small molecule inhibitor, a chemotherapeutic agent, a cancer a targeted therapy (e.g., immunotherapy), an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

In some embodiments, the chemotherapeutic agent of the system described herein comprises one or more of an alkylating agent, an alkyl sulfonates aziridine, an ethylenimine, a methylamelamine, an acetogenin, a camptothecin, a bryostatin, a callystatin, CC-1065, a cryptophycin, aa dolastatin, a duocarmycin, a eleutherobin, a pancratistatin, a sarcodictyin, a spongistatin, a nitrogen mustard, a nitrosureas, an antibiotic, a dynemicin, a bisphosphonate, an esperamicina a neocarzinostatin chromophore or a related chromoprotein enediyne antibiotic chromophore, an anti-metabolite, a folic acid analogue, a purine analog, a pyrimidine analog, an androgens, an anti-adrenal, a folic acid replenisher, aldophosphamide glycoside, aminolevulinic acid, eniluracil, amsacrine, bestrabucil, bisantrene, edatraxate, defofamine, demecolcine, diaziquone, elformithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, a PSK polysaccharide complex, razoxane, rhizoxin, sizofiran, spirogermanium, tenuazonic acid, triaziquone, 2,2′,2″-trichlorotriethylamine, a trichothecene, urethan, vindesine, dacarbazine, mannomustine, mitobronitol, mitolactol, pipobroman, gacytosine, arabinoside (“Ara-C”), cyclophosphamide, a taxoid, 6-thioguanine, mercaptopurine, a platinum coordination complex, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluorometlhylomithine (DMFO), a retinoid, capecitabine, carboplatin, procarbazine, plicomycin, gemcitabine, navelbine, farnesyl-protein transferase inhibitors, transplatinum, or any combination thereof.

In some embodiments, the targeted therapy (or anti-cancer target therapy) of the system described herein may comprise abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aligopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some embodiments, the cancer of the system described herein is a B cell cancer, a melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer or carcinoma, lung non-small cell lung carcinoma (NSCLC), head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the subject of the system described herein is a human. In some embodiments, the subject has previously been treated with an anti-cancer therapy. In some embodiments, the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLiD) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for determining clonal tumor mutational burden (cTMB) in any of a variety of samples as described herein (e.g., a tissue sample, biopsy sample, hematological sample, or liquid biopsy sample derived from the subject).

In some instances, the plurality of gene loci for which sequencing data is processed to determine clonal tumor mutational burden (cTMB) may comprise at least 1, 2, 3, 4, 5, 6, 7, 8, 9, 10, or more than 10 gene loci.

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of clonal tumor mutational burden (cTMB) is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### XIX. COMPUTER SYSTEMS AND NETWORKS

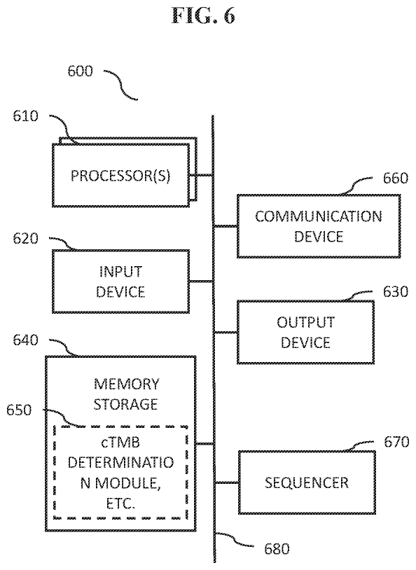

FIG. 6 illustrates an example of a computing device or system in accordance with one embodiment. Device 600 can be a host computer connected to a network. Device 600 can be a client computer or a server. As shown in FIG. 6, device 600 can be any suitable type of microprocessor-based device, such as a personal computer, workstation, server or handheld computing device (portable electronic device) such as a phone or tablet. The device can include, for example, one or more processor(s) 610, input devices 620, output devices 630, memory or storage devices 640, communication devices 660, and nucleic acid sequencers 670. Software 650 residing in memory or storage device 640 may comprise, e.g., an operating system as well as software for executing the methods described herein. Input device 620 and output device 630 can generally correspond to those described herein, and can either be connectable or integrated with the computer.

Input device 620 can be any suitable device that provides input, such as a touch screen, keyboard or keypad, mouse, or voice-recognition device. Output device 630 can be any suitable device that provides output, such as a touch screen, haptics device, or speaker.

Storage 640 can be any suitable device that provides storage (e.g., an electrical, magnetic or optical memory including a RAM (volatile and non-volatile), cache, hard drive, or removable storage disk). Communication device 660 can include any suitable device capable of transmitting and receiving signals over a network, such as a network interface chip or device. The components of the computer can be connected in any suitable manner, such as via a wired media (e.g., a physical system bus 680, Ethernet connection, or any other wire transfer technology) or wirelessly (e.g., Bluetooth®, Wi-Fi®, or any other wireless technology).

Software module 650, which can be stored as executable instructions in storage 640 and executed by processor(s) 610, can include, for example, an operating system and/or the processes that embody the functionality of the methods of the present disclosure (e.g., as embodied in the devices as described herein).

Software module 650 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 640, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 650 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

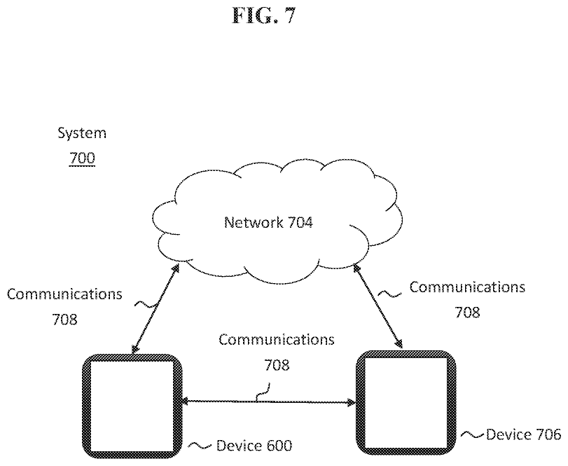

Device 600 may be connected to a network (e.g., network 704, as shown in FIG. 7 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 600 can be implemented using any operating system, e.g., an operating system suitable for operating on the network. Software module 650 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 610.

Device 600 can further include a sequencer 670, which can be any suitable nucleic acid sequencing instrument.

FIG. 7 illustrates an example of a computing system in accordance with one embodiment. In system 700, device 600 (e.g., as described above and illustrated in FIG. 6) is connected to network 704, which is also connected to device 706. In some embodiments, device 706 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio® RS system.

Devices 600 and 706 may communicate, e.g., using suitable communication interfaces via network 704, such as a Local Area Network (LAN), Virtual Private Network (VPN), or the Internet. In some embodiments, network 704 can be, for example, the Internet, an intranet, a virtual private network, a cloud network, a wired network, or a wireless network. Devices 600 and 706 may communicate, in part or in whole, via wireless or hardwired communications, such as Ethernet, IEEE 802.11b wireless, or the like. Additionally, devices 600 and 706 may communicate, e.g., using suitable communication interfaces, via a second network, such as a mobile/cellular network. Communication between devices 600 and 706 may further include or communicate with various servers such as a mail server, mobile server, media server, telephone server, and the like. In some embodiments, Devices 600 and 706 can communicate directly (instead of, or in addition to, communicating via network 704), e.g., via wireless or hardwired communications, such as Ethernet, IEEE 802.11b wireless, or the like. In some embodiments, devices 600 and 706 communicate via communications 708, which can be a direct connection or can occur via a network (e.g., network 704).

One or all of devices 600 and 706 generally include logic (e.g., http web server logic) or are programmed to format data, accessed from local or remote databases or other sources of data and content, for providing and/or receiving information via network 704 according to various examples described herein.

### EXEMPLARY EMBODIMENTS

The following examples are exemplary and are not intended to limit the scope of the claimed invention.

Embodiment 1. A method comprising:
obtaining, using one or more processors, a plurality of sequence read data that represent a set of nucleic acid molecules obtained from a sample for a subject;
identifying, using the one or more processors, one or more short variants from the sequence read data;
filtering, using the one or more processors, the one or more short variants to select one or more clonal tumor mutational burden (cTMB)-qualified short variants, wherein the filtering comprises excluding short variants identified as subclonal based on a tumor amount measure for a respective short variant; and
determining, using the one or more processors, a cTMB value for the sample based on the number of cTMB-qualified short variants.

Embodiment 2. A method comprising:
receiving, at one or more processors, genomic data for a subject, wherein the genomic data indicates a presence or absence of one or more short variants in a sample derived from the subject;
filtering, using the one or more processors, the one or more short variants to select one or more clonal tumor mutational burden (cTMB)-qualified short variants, wherein the filtering comprises excluding short variants identified as subclonal based on a tumor amount measure for a respective short variant; and
determining, using the one or more processors, a cTMB value for the sample based on the number of cTMB-qualified short variants.

Embodiment 3. The method of any of embodiment 1 or embodiment 2, wherein the subject has a cancer.

Embodiment 4. The method of any one of embodiments 1-3, wherein the cTMB is determined with one or more of (a) counting a number of the one or more clonal short variants in the sample, and (b) normalizing a size of a bait molecule.

Embodiment 5. The method of any one of embodiments 1-4, wherein the tumor amount measure comprises a cancer cell fraction (CCF) of the short variant or a variant allele frequency (VAF).

Embodiment 6. The method of any one of embodiments 1-5, wherein the filtering comprises excluding short variants identified as having an allele frequency below a predetermined threshold.

Embodiment 7. The method of any one of embodiments 1-6, wherein the filtering comprises excluding short variants identified as a germline variant.

Embodiment 8. The method of any one of embodiments 1-7, wherein the filtering comprises excluding short variants identified as having a cancer cell fraction (CCF) at or below a predetermined CCF threshold.

Embodiment 9. The method of any one of embodiments 1-8, wherein a clonal tumor mutational burden (cTMB) value is expressed as a function of the number of cTMB-qualified short variants per megabase (mutations/Mb) in a set of subgenomic intervals from the sample.

Embodiment 10. The method of embodiment 9, wherein the set of subgenomic intervals from the sample are between about 100 kb to about 10 Mb.

Embodiment 11. The method of embodiment 9 or embodiment 10, wherein the set of subgenomic intervals from the sample are between about 0.8 Mb to about 1.1 Mb.

Embodiment 12. The method of any one of embodiments 1-11, wherein the genomic data for the subject is based on a targeted exome sequencing panel.

Embodiment 13. The method of any one of embodiments 1-12, wherein the genomic data for the subject is derived from a single biopsy sample.

Embodiment 14. The method of any one of embodiments 1-13, wherein the genomic data for the subject is derived from only one biopsy sample.

Embodiment 15. The method of any one of embodiments 1-13, wherein the genomic data for the subject is derived from circulating tumor DNA in a liquid biopsy sample.

Embodiment 16. The method of any one of embodiments 1-13, wherein the genomic data for the subject is derived from single cell sequencing.

Embodiment 17. The method of any one of embodiments 1-16, wherein the one or more short variants include noncoding and synonymous short variants.

Embodiment 18. The method of any one of embodiments 1-17, further comprising obtaining the sample from the subject.

Embodiment 19. The method of any one of embodiments 1-18, wherein the sample comprises a tissue biopsy sample, a liquid biopsy sample, or a normal control.

Embodiment 20. The method of embodiment 19, wherein the sample is a liquid biopsy sample and comprises blood, plasma, cerebrospinal fluid, sputum, stool, urine, or saliva.

Embodiment 21. The method of embodiment 19, wherein the sample is a liquid biopsy sample and comprises circulating tumor cells (CTCs).

Embodiment 22. The method of embodiment 19, wherein the sample is a liquid biopsy sample and comprises cell-free DNA (cfDNA), circulating tumor DNA (ctDNA), or any combination thereof.

Embodiment 23. The method of any one of embodiments 1-22, wherein the plurality of nucleic acid molecules comprises a mixture of tumor nucleic acid molecules and non-tumor nucleic acid molecules.

Embodiment 24. The method of embodiment 23, wherein the tumor nucleic acid molecules are derived from a tumor portion of a heterogeneous tissue biopsy sample, and the non-tumor nucleic acid molecules are derived from a normal portion of the heterogeneous tissue biopsy sample.

Embodiment 25. The method of embodiment 23, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

Embodiment 26. The method of any one of embodiments 1-25, wherein the genomic data is obtained from sequencing the sample.

Embodiment 27. The method of embodiment 26, wherein the sequencing comprises use of a massively parallel sequencing (MPS) technique, whole genome sequencing (WGS), whole exome sequencing, targeted sequencing, direct sequencing, or Sanger sequencing technique.

Embodiment 28. The method of embodiment 27, wherein the sequencing comprises massively parallel sequencing, and the massively parallel sequencing technique comprises next generation sequencing (NGS).

Embodiment 29. The method of any one of embodiments 26-28, wherein the sequencing comprises:
providing a plurality of nucleic acid molecules obtained from the sample from the subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the set of nucleic acid molecules in the sample.

Embodiment 30. The method of embodiment 29, wherein the one or more adapters comprise amplification primers, flow cell adapter sequences, unique molecular identifier sequence, substrate adapter sequences, or sample index sequences.

Embodiment 31. The method of embodiment 29 or embodiment 30, wherein the captured nucleic acid molecules are captured from the amplified nucleic acid molecules by hybridization to one or more bait molecules.

Embodiment 32. The method of embodiment 31, wherein the one or more bait molecules comprise one or more nucleic acid molecules, each comprising a region that is complementary to a region of a captured nucleic acid molecule.

Embodiment 33. The method of any one of embodiments 29-32, wherein amplifying nucleic acid molecules comprises performing a polymerase chain reaction (PCR) amplification technique, a non-PCR amplification technique, or an isothermal amplification technique.

Embodiment 34. The method of any one of embodiments 1-33, wherein the plurality of sequence reads overlap one or more gene loci within a subgenomic interval in the sample.

Embodiment 35. The method of any one of embodiments 1-34, wherein the one or more short variants comprise ALOX12B T402T, ATRX K2225del, CTNNA1 D362N, FLT3 S102S, JAK2 I289V, KEAP1 K97N, KRAS Q61H, MSH3 R573fs*4, PDK1 T306M, STAG2 C527F, STK11 649_650delCC, TSC2 A68S, or any combination thereof.

Embodiment 36. The method of any one of embodiments 1-35, wherein the one or more cTMB-qualified short variants comprise CTNNA1 D362N, FLT3 S102S, STAG2 C527F, TSC2 A68S, or any combination thereof.

Embodiment 37. The method of any one of embodiments 1-36, further comprising generating, by the one or more processors, a report comprising a cTMB for the subject.

Embodiment 38. The method of embodiment 37, further comprising transmitting the report to a healthcare provider.

Embodiment 39. The method of embodiment 38, wherein the report is transmitted via a computer network or a peer-to-peer connection.

Embodiment 40. A method of treating a subject having a cancer comprising:
(a) determining a clonal tumor mutational burden (cTMB) value in a sample from the subject by the method of any one of embodiments 1-36; and
(b) treating the subject with an immuno-oncology (IO) therapy if the cTMB value determined for the sample is at or above a threshold cTMB value.

Embodiment 41. A method of selecting a treatment for a subject having a cancer, the method comprising determining a clonal tumor mutational burden (cTMB) value in a sample from the subject by the method of any one of embodiments 1-36, wherein if the cTMB value determined for the sample is at or above a threshold cTMB value, the subject is identified as one who may benefit from treatment with an immune-oncology (IO) therapy.

Embodiment 42. A method of identifying a subject having a cancer for treatment with an immune-oncology (IO) therapy comprising:
(a) determining a clonal tumor mutational burden (cTMB) value in a sample from the subject by the method of any one of embodiments 1-36; and
(b) identifying the subject for treatment with the IO therapy if the cTMB value determined for the sample is at or above a threshold cTMB value.

Embodiment 43. A method of identifying one or more treatment options for a subject having a cancer, the method comprising:
(a) determining a clonal tumor mutational burden (cTMB) value in a sample from the subject by the method of any one of embodiments 1-36; and
(b) generating a report comprising one or more treatment options identified for the subject based at least in part on the cTMB value determined for the sample, wherein if the cTMB value is at or above a threshold cTMB value, the subject is identified as one who may benefit from treatment with an immune-oncology (IO) therapy.

Embodiment 44. A method of predicting survival of a subject having cancer, the method comprising determining a clonal tumor mutational burden (cTMB) value in a sample from the subject by the method of any one of embodiments 1-36, wherein if the cTMB value determined for the sample is at or above a threshold cTMB value, the subject is predicted to have longer survival when treated with an immune-oncology (IO) therapy, as compared to a subject determined to have a cTMB value below the threshold cTMB value.

Embodiment 45. The method of embodiment any one of embodiments 40-44, wherein the threshold cTMB value is at least about 4 to 100 mutations/Mb, about 4 to 30 mutations/Mb, 8 to 100 mutations/Mb, 8 to 30 mutations/Mb, 10 to 20 mutations/Mb, less than 4 mutations/Mb, or less than 8 mutations/Mb.

Embodiment 46. The method of embodiment 45, wherein the threshold cTMB value is at least about 5 mutations/Mb, at least about 10 mutations/Mb, at least about 12 mutations/Mb, at least about 16 mutations/Mb, at least about 20 mutations/Mb, or at least about 30 mutations/Mb.

Embodiment 47. The method of embodiment 45, wherein the threshold cTMB value is at least about 7.5 mutations/Mb, at least about 8.5 mutations/Mb, at least about 9.5 mutations/Mb, at least about 11 mutations/Mb, at least about 13.5 mutations/Mb, at least about 16.5 mutations/Mb, at least about 18.5 mutations/Mb, at least about 19 mutations/Mb, or at least about 25 mutations/Mb.

Embodiment 48. The method of any one of embodiments 40-47, wherein the immuno-oncology (IO) therapy comprises a single IO agent or multiple IO agents.

Embodiment 49. The method of any one of embodiments 40-48, wherein the immuno-oncology (IO) therapy comprises an immune checkpoint inhibitor.

Embodiment 50. The method of embodiment 49, wherein the immune checkpoint inhibitor comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a cellular therapy, a treatment for cancer being tested in a clinical trial, an immunotherapy, or any combination thereof.

Embodiment 51. The method of embodiment 49 or embodiment 50, wherein the immune checkpoint inhibitor is a PD-1 inhibitor.

Embodiment 52. The method of embodiment 51, wherein the immune checkpoint inhibitor comprises one or more of nivolumab, pembrolizumab, cemiplimab, or dostarlimab.

Embodiment 53. The method of embodiment 49 or embodiment 50, wherein the immune checkpoint inhibitor is a PD-L1-inhibitor.

Embodiment 54. The method of embodiment 53, wherein the immune checkpoint inhibitor comprises one or more of atezolizumab, avelumab, or durvalumab.

Embodiment 55. The method of embodiment 49 or embodiment 50, wherein the immune checkpoint inhibitor is a CTLA-4 inhibitor.

Embodiment 56. The method of embodiment 55, wherein the CTLA-4 inhibitor comprises ipilimumab.

Embodiment 57. The method of embodiment 49 or embodiment 50, wherein the nucleic acid comprises a double-stranded RNA (dsRNA), a small interfering RNA (siRNA), or a small hairpin RNA (shRNA).

Embodiment 58. The method of embodiment 49 or embodiment 50, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Embodiment 59. The method of any one of embodiments 40-58, further comprising treating the subject with the IO therapy.

Embodiment 60. The method of any one of embodiments 40-59, further comprising treating the subject with an additional anti-cancer therapy.

Embodiment 61. The method of embodiment 60, wherein the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

Embodiment 62. The method of embodiment 61, wherein the chemotherapeutic agent comprises one or more of an alkylating agent, an alkyl sulfonates aziridine, an ethylenimine, a methylamelamine, an acetogenin, a camptothecin, a bryostatin, a callystatin, CC-1065, a cryptophycin, aa dolastatin, a duocarmycin, a eleutherobin, a pancratistatin, a sarcodictyin, a spongistatin, a nitrogen mustard, a nitrosureas, an antibiotic, a dynemicin, a bisphosphonate, an esperamicina a neocarzinostatin chromophore or a related chromoprotein enediyne antibiotic chromophore, an anti-metabolite, a folic acid analogue, a purine analog, a pyrimidine analog, an androgens, an anti-adrenal, a folic acid replenisher, aldophosphamide glycoside, aminolevulinic acid, eniluracil, amsacrine, bestrabucil, bisantrene, edatraxate, defofamine, demecolcine, diaziquone, elformithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, a PSK polysaccharide complex, razoxane, rhizoxin, sizofiran, spirogermanium, tenuazonic acid, triaziquone, 2,2′,2″-trichlorotriethylamine, a trichothecene, urethan, vindesine, dacarbazine, mannomustine, mitobronitol, mitolactol, pipobroman, gacytosine, arabinoside (“Ara-C”), cyclophosphamide, a taxoid, 6-thioguanine, mercaptopurine, a platinum coordination complex, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluorometlhylomithine (DMFO), a retinoid, capecitabine, carboplatin, procarbazine, plicomycin, gemcitabine, navelbine, farnesyl-protein transferase inhibitors, transplatinum, or any combination thereof.

Embodiment 63. The method of any one of embodiments 3-62, wherein the cancer is a B cell cancer, a melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer or carcinoma, lung non-small cell lung carcinoma (NSCLC), head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

Embodiment 64. The method of any of embodiments 1-63, wherein the subject is a human.

Embodiment 65. The method of any one of embodiments 1-64, wherein the subject has previously been treated with an anti-cancer therapy.

Embodiment 66. The method of embodiment 65, wherein the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

Embodiment 67. A system comprising:
one or more processors, and
a memory communicatively coupled to the one or more processors and configured to store instructions that, when executed by the one or more processors, cause the system to:
   receive a plurality of sequence read data that represent a set of nucleic acid molecules obtained from a sample for a subject;
   identify one or more short variants from the sequence read data;
   filter the one or more short variants to select one or more clonal tumor mutational burden (cTMB)-qualified short variants, wherein the filtering comprises excluding short variants identified as subclonal based on a tumor amount measure for a respective short variant; and
   determine a cTMB value for the sample based on the number of cTMB-qualified short variants.

Embodiment 68. A system comprising:
one or more processors, and
a memory communicatively coupled to the one or more processors and configured to store instructions that, when executed by the one or more processors, cause the system to:
   receive genomic data for a subject, wherein the genomic data indicates a presence or absence of one or more short variants in a sample derived from the subject;
   filter the one or more short variants to select one or more clonal tumor mutational burden (cTMB)-qualified short variants, wherein the filtering comprises excluding short variants identified as subclonal based on a tumor amount measure for a respective short variant; and
   determine a cTMB value for the sample based on the number of cTMB-qualified short variants.

Embodiment 69. The system of embodiment 67 or embodiment 68, wherein the instructions further cause the system to select a treatment for the subject, wherein the subject has a cancer, based on the cTMB for the sample.

Embodiment 70. The system of embodiment 69, wherein if the cTMB determined for the sample is at or above a threshold cTMB, the subject is identified as one who may benefit from treatment with an immune-oncology (IO) therapy.

Embodiment 71. The system of any one of embodiments 67-70, wherein the instructions further cause the system to generate a report comprising one or more treatment options identified for the subject based at least in part on the cTMB determined for the sample, wherein if the cTMB is at or above a threshold cTMB, the subject is identified as one who may benefit from treatment with an immune-oncology (IO) therapy.

Embodiment 72. The system of any one of embodiments 67-71, wherein the instructions further cause the system to predict survival of the subject, wherein the subject has cancer, wherein if the cTMB determined for the sample is at or above a threshold cTMB, the subject is predicted to have longer survival when treated with an immune-oncology (IO) therapy, as compared to a subject determined to have a cTMB below the threshold cTMB.

Embodiment 73. The system of any one of embodiments 67-72, wherein the cTMB is determined with one or more of (a) counting a number of the one or more clonal short variants in the sample, and (b) normalizing a size of a bait molecule.

Embodiment 74. The system of any one of embodiments 67-73, wherein the tumor amount measure comprises a cancer cell fraction (CCF) of the short variant or a variant allele frequency (VAF).

Embodiment 75. The system of any one of embodiments 67-74, wherein the filter step comprises excluding short variants identified as having an allele frequency below a predetermined threshold.

Embodiment 76. The system of any one of embodiments 67-75, wherein the filter step comprises excluding short variants identified as a germline variant.

Embodiment 77. The system of any one of embodiments 67-76, wherein the filter step comprises excluding short variants identified as having a cancer cell fraction (CCF) at or below a predetermined CCF threshold.

Embodiment 78. The system of any one of embodiments 67-77, wherein a clonal tumor mutational burden (cTMB) value is expressed as a function of the number of cTMB-qualified short variants per megabase (mutations/Mb) in a set of subgenomic intervals from the sample.

Embodiment 79. The system of embodiment 78, wherein the set of subgenomic intervals from the sample are between about 100 kb to about 10 Mb.

Embodiment 80. The system of embodiment 78 or embodiment 79, wherein the set of subgenomic intervals from the sample are between about 0.8 Mb to about 1.1 Mb.

Embodiment 81. The system of any one of embodiments 67-80, wherein the genomic data for the subject is based on a targeted exome sequencing panel.

Embodiment 82. The system of any one of embodiments 67-81, wherein the genomic data for the subject is derived from a single biopsy sample.

Embodiment 83. The system of any one of embodiments 67-82, wherein the genomic data for the subject is derived from only one biopsy sample.

Embodiment 84. The system of any one of embodiments 67-83, wherein the genomic data for the subject is derived from circulating tumor DNA in a liquid biopsy sample.

Embodiment 85. The system of any one of embodiments 67-83, wherein the genomic data for the subject is derived from single cell sequencing.

Embodiment 86. The system of any one of embodiments 67-85, wherein the one or more short variants include noncoding and synonymous short variants.

Embodiment 87. The system of any one of embodiments 67-86, further comprising obtaining the sample from the subject.

Embodiment 88. The system of any one of embodiments 67-87, wherein the sample comprises a tissue biopsy sample, a liquid biopsy sample, or a normal control.

Embodiment 89. The system of embodiment 88, wherein the sample is a liquid biopsy sample and comprises blood, plasma, cerebrospinal fluid, sputum, stool, urine, or saliva.

Embodiment 90. The system of embodiment 88, wherein the sample is a liquid biopsy sample and comprises circulating tumor cells (CTCs).

Embodiment 91. The system of embodiment 88, wherein the sample is a liquid biopsy sample and comprises cell-free DNA (cfDNA), circulating tumor DNA (ctDNA), or any combination thereof.

Embodiment 92. The system of any one of embodiments 67-91, wherein the plurality of nucleic acid molecules comprises a mixture of tumor nucleic acid molecules and non-tumor nucleic acid molecules.

Embodiment 93. The system of embodiment 92, wherein the tumor nucleic acid molecules are derived from a tumor portion of a heterogeneous tissue biopsy sample, and the non-tumor nucleic acid molecules are derived from a normal portion of the heterogeneous tissue biopsy sample.

Embodiment 94. The system of embodiment 92, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

Embodiment 95. The system of any one of embodiments 67-94, wherein the genomic data is obtained from sequencing the sample.

Embodiment 96. The system of embodiment 95, wherein the sequencing comprises use of a massively parallel sequencing (MPS) technique, whole genome sequencing (WGS), whole exome sequencing, targeted sequencing, direct sequencing, or Sanger sequencing technique.

Embodiment 97. The system of embodiment 96, wherein the sequencing comprises massively parallel sequencing, and the massively parallel sequencing technique comprises next generation sequencing (NGS).

Embodiment 98. The system of any one of embodiments 95-97, wherein the sequencing comprises:
providing a plurality of nucleic acid molecules obtained from the sample from the subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the set of nucleic acid molecules in the sample.

Embodiment 99. The system of embodiment 98, wherein the one or more adapters comprise amplification primers, flow cell adapter sequences, unique molecular identifier sequence, substrate adapter sequences, or sample index sequences.

Embodiment 100. The system of embodiment 98 or embodiment 99, wherein the captured nucleic acid molecules are captured from the amplified nucleic acid molecules by hybridization to one or more bait molecules.

Embodiment 101. The system of embodiment 100, wherein the one or more bait molecules comprise one or more nucleic acid molecules, each comprising a region that is complementary to a region of a captured nucleic acid molecule.

Embodiment 102. The system of any one of embodiments 98-101, wherein amplifying nucleic acid molecules comprises performing a polymerase chain reaction (PCR) amplification technique, a non-PCR amplification technique, or an isothermal amplification technique.

Embodiment 103. The system of any one of embodiments 67-102, wherein the plurality of sequence reads overlap one or more gene loci within a subgenomic interval in the sample.

Embodiment 104. The system of any one of embodiments 67-103, wherein the one or more short variants comprise ALOX12B T402T, ATRX K2225del, CTNNA1 D362N, FLT3 S102S, JAK2 I289V, KEAP1 K97N, KRAS Q61H, MSH3 R573fs*4, PDK1 T306M, STAG2 C527F, STK11 649_650delCC, TSC2 A68S, or any combination thereof.

Embodiment 105. The system of any one of embodiments 67-104, wherein the one or more cTMB-qualified short variants comprise CTNNA1 D362N, FLT3 S102S, STAG2 C527F, TSC2 A68S, or any combination thereof.

Embodiment 106. The system of any one of embodiments 67-105, further comprising generating, by the one or more processors, a report comprising a cTMB for the subject.

Embodiment 107. The system of embodiment 106, further comprising transmitting the report to a healthcare provider.

Embodiment 108. The system of embodiment 107, wherein the report is transmitted via a computer network or a peer-to-peer connection.

Embodiment 109. The system of any one of embodiments 67-108, wherein a threshold cTMB value is at least about 4 to 100 mutations/Mb, about 4 to 30 mutations/Mb, 8 to 100 mutations/Mb, 8 to 30 mutations/Mb, 10 to 20 mutations/Mb, less than 4 mutations/Mb, or less than 8 mutations/Mb.

Embodiment 110. The system of any one of embodiments 67-108, wherein a threshold cTMB value is at least about 5 mutations/Mb, at least about 10 mutations/Mb, at least about 12 mutations/Mb, at least about 16 mutations/Mb, at least about 20 mutations/Mb, or at least about 30 mutations/Mb.

Embodiment 111. The system of any one of embodiments 67-108, wherein a threshold cTMB value is at least about 7.5 mutations/Mb, at least about 8.5 mutations/Mb, at least about 9.5 mutations/Mb, at least about 11 mutations/Mb, at least about 13.5 mutations/Mb, at least about 16.5 mutations/Mb, at least about 18.5 mutations/Mb, at least about 19 mutations/Mb, or at least about 25 mutations/Mb.

Embodiment 112. The system of any one of embodiments 67-111, wherein the immuno-oncology (IO) therapy comprises a single IO agent or multiple IO agents.

Embodiment 113. The system of any one of embodiments 67-112, wherein the immuno-oncology (IO) therapy comprises an immune checkpoint inhibitor.

Embodiment 114. The system of embodiment 113, wherein the immune checkpoint inhibitor comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a cellular therapy, a treatment for cancer being tested in a clinical trial, an immunotherapy, or any combination thereof.

Embodiment 115. The system of embodiment 113 or embodiment 114, wherein the immune checkpoint inhibitor is a PD-1 inhibitor.

Embodiment 116. The system of embodiment 115, wherein the immune checkpoint inhibitor comprises one or more of nivolumab, pembrolizumab, cemiplimab, or dostarlimab.

Embodiment 117. The system of embodiment 113 or embodiment 114, wherein the immune checkpoint inhibitor is a PD-L1-inhibitor.

Embodiment 118. The system of embodiment 117, wherein the immune checkpoint inhibitor comprises one or more of atezolizumab, avelumab, or durvalumab.

Embodiment 119. The system of embodiment 113 or embodiment 114, wherein the immune checkpoint inhibitor is a CTLA-4 inhibitor.

Embodiment 120. The system of embodiment 119, wherein the CTLA-4 inhibitor comprises ipilimumab.

Embodiment 121. The system of embodiment 113 or embodiment 114, wherein the nucleic acid comprises a double-stranded RNA (dsRNA), a small interfering RNA (siRNA), or a small hairpin RNA (shRNA).

Embodiment 122. The system of embodiment 113 or embodiment 114, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Embodiment 123. The system of any one of embodiments 67-122, further comprising treating the subject with the IO therapy.

Embodiment 124. The system of any one of embodiments 67-123, further comprising treating the subject with an additional anti-cancer therapy.

Embodiment 125. The system of embodiment 124, wherein the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

Embodiment 126. The system of embodiment 125, wherein the chemotherapeutic agent comprises one or more of an alkylating agent, an alkyl sulfonates aziridine, an ethylenimine, a methylamelamine, an acetogenin, a camptothecin, a bryostatin, a callystatin, CC-1065, a cryptophycin, aa dolastatin, a duocarmycin, a eleutherobin, a pancratistatin, a sarcodictyin, a spongistatin, a nitrogen mustard, a nitrosureas, an antibiotic, a dynemicin, a bisphosphonate, an esperamicina a neocarzinostatin chromophore or a related chromoprotein enediyne antibiotic chromophore, an anti-metabolite, a folic acid analogue, a purine analog, a pyrimidine analog, an androgens, an anti-adrenal, a folic acid replenisher, aldophosphamide glycoside, aminolevulinic acid, eniluracil, amsacrine, bestrabucil, bisantrene, edatraxate, defofamine, demecolcine, diaziquone, elformithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, a PSK polysaccharide complex, razoxane, rhizoxin, sizofiran, spirogermanium, tenuazonic acid, triaziquone, 2,2′,2″-trichlorotriethylamine, a trichothecene, urethan, vindesine, dacarbazine, mannomustine, mitobronitol, mitolactol, pipobroman, gacytosine, arabinoside (“Ara-C”), cyclophosphamide, a taxoid, 6-thioguanine, mercaptopurine, a platinum coordination complex, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluorometlhylomithine (DMFO), a retinoid, capecitabine, carboplatin, procarbazine, plicomycin, gemcitabine, navelbine, farnesyl-protein transferase inhibitors, transplatinum, or any combination thereof.

Embodiment 127. The system of any one of embodiments 67-126, wherein the cancer is a B cell cancer, a melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer or carcinoma, lung non-small cell lung carcinoma (NSCLC), head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

Embodiment 128. The system of any one of embodiments 67-127, wherein the subject is a human.

Embodiment 129. The system of any one of embodiments 67-128, wherein the subject has previously been treated with an anti-cancer therapy.

Embodiment 130. The system of embodiment 129, wherein the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

### Examples

The invention will be more fully understood by reference to the following examples. They should not, however, be construed as limiting the scope of the invention. It is understood that the examples and embodiments described herein are for illustrative purposes only, and that various modifications or changes in light thereof will be suggested to persons skilled in the art and are to be included within the spirit and purview of this application and scope of the appended claims.

### Example 1: Deriving Clonal Tumor Mutational Burden (cTMB) from a Non-Small Cell Lung Cancer Clinico-Genomic Database Specimen

This Example describes a workflow to investigate existing genomic data from a non-small cell lung cancer (NSCLC) clinico-genomic database (CGDB) using the method described herein for filtering genomic data for clonal variants associated with cTMB and improved immunotherapy response in patients.

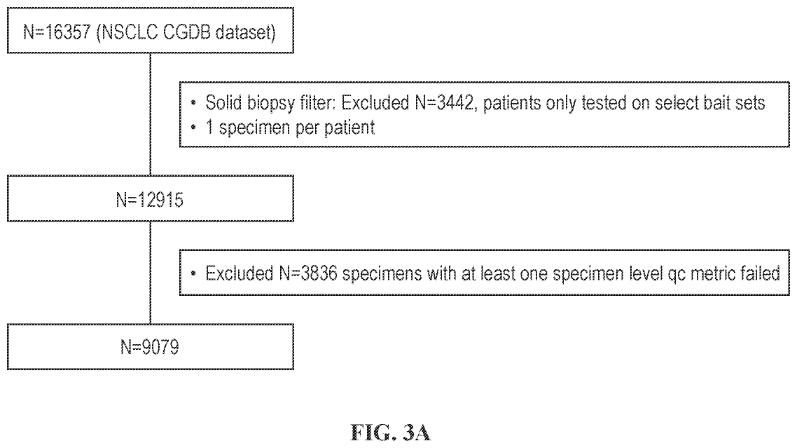

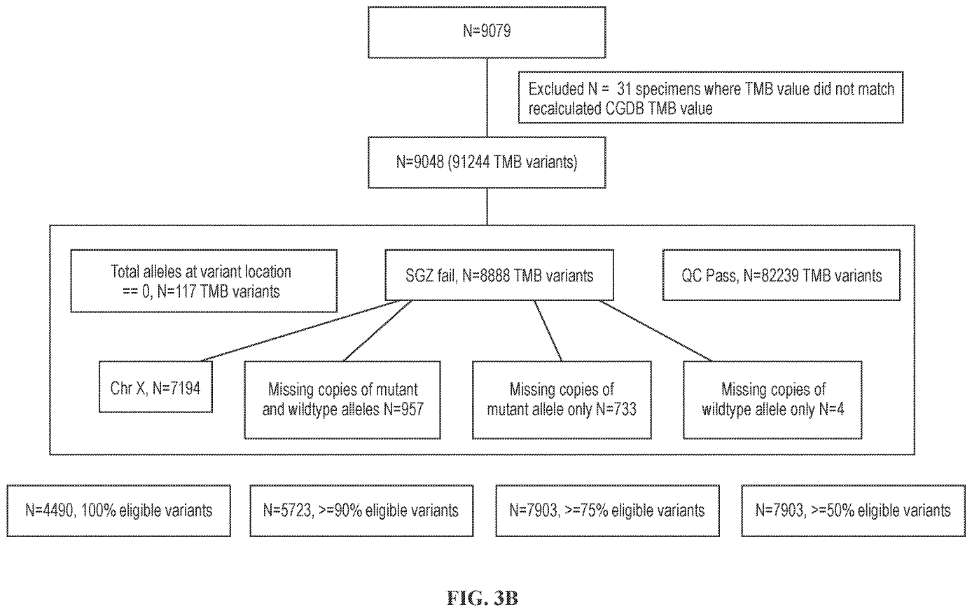

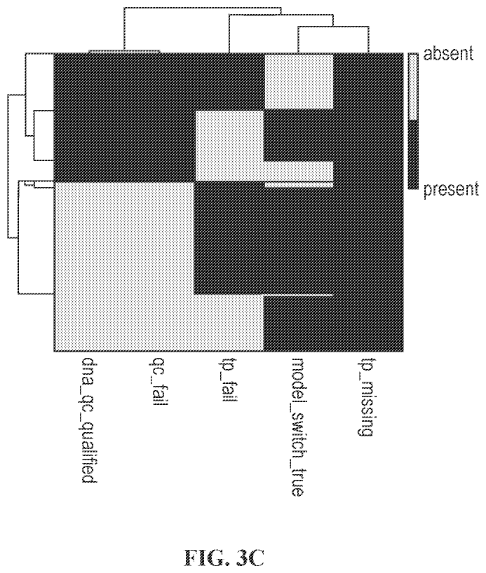

As shown in the study design/cohort diagram of FIG. 3A and FIG. 3B, a cohort of non-small cell lung cancer (NSCLC) patient samples from a clinico-genomic database (CGDB) were used in evaluating clonal tumor mutational burden (cTMB) as a biomarker for the patient's response to immune checkpoint inhibitors (ICI) therapy, in accordance with some instances of the methods and systems described herein. FIG. 3A showed the first part of the study design and FIG. 3B showed the second part of the study. Using a non-small cell lung cancer (NSCLC) clinic-genomic database (CGDB), 16,357 specimen were investigated (FIG. 3A) in Part 1. Filtering for solid biopsy specimens, patients only tested using certain predefined bait sets, and requiring only one specimen per patient, the sample set was narrowed to 12,915 specimen. From this, specimens with at least one failed specimen level quality control (QC) metric were removed, as shown in FIG. 3C, narrowing the sample set was narrowed to 9,079 specimen. After re-calculating TMB from short variant level data within the CGDB, 31 specimen were found to have unmatching TMB (e.g., re-calculated CGDB TMB was not equal to TMB research use only (RUO) value from the CGDB's complex signatures table) and were removed, providing a set of 9048 specimen having 91244 TMB variants.

Variants were then assessed for a set of variant-level quality control criteria (FIG. 3B). Variants in copy number regions that were not well-modeled (N=117) were removed from the analysis. TMB variants present on chromosome X (N=7194), had missing copies of mutant and wild-type alleles (N=957), had missing copies of mutant alleles only (N=733), and had missing copies of wild-type alleles only (N=4) were identified.

From this, 82,239 TMB variants passed the quality control analysis. Based on the above described variant level quality control analyses, 4490 specimens had 100% TMB variants (i.e., all of their TMB variants) eligible for CCF estimation, 5723 specimens had at least 90% TMB variants eligible for CCF estimation, 7903 specimens had at least 75% TMB variants eligible CCF estimation, and 8863 specimens had at least 50% TMB variants eligible for CCF estimation (FIG. 3B). TMB-qualified short variants were then filtered for CCF, where, for example, the CCF was greater than or equal to 0.5, to determine one or more clonal TMB (cTMB)-qualified variants.

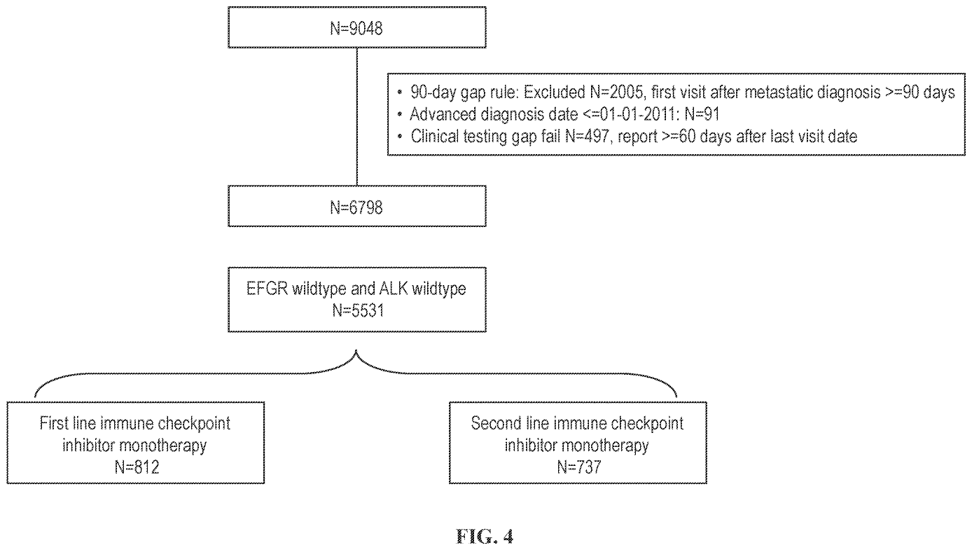

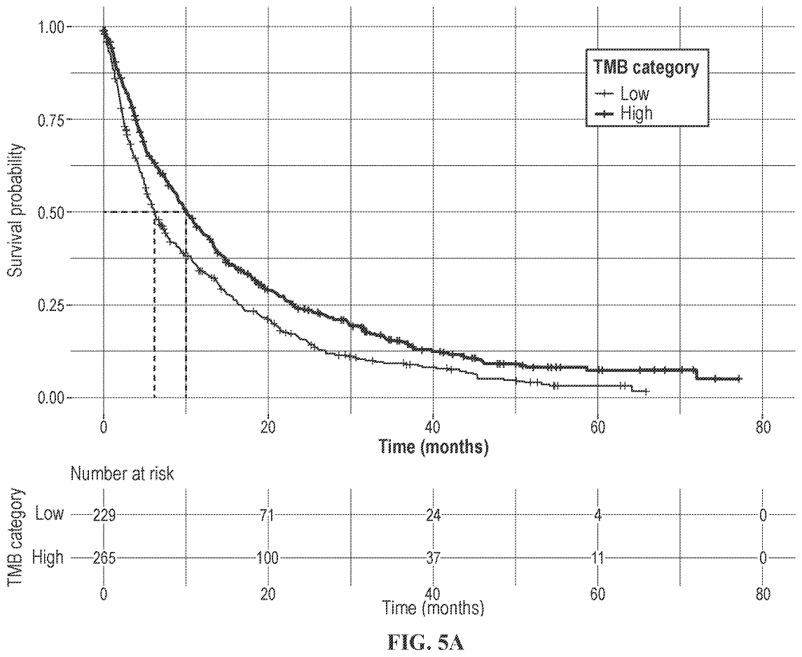

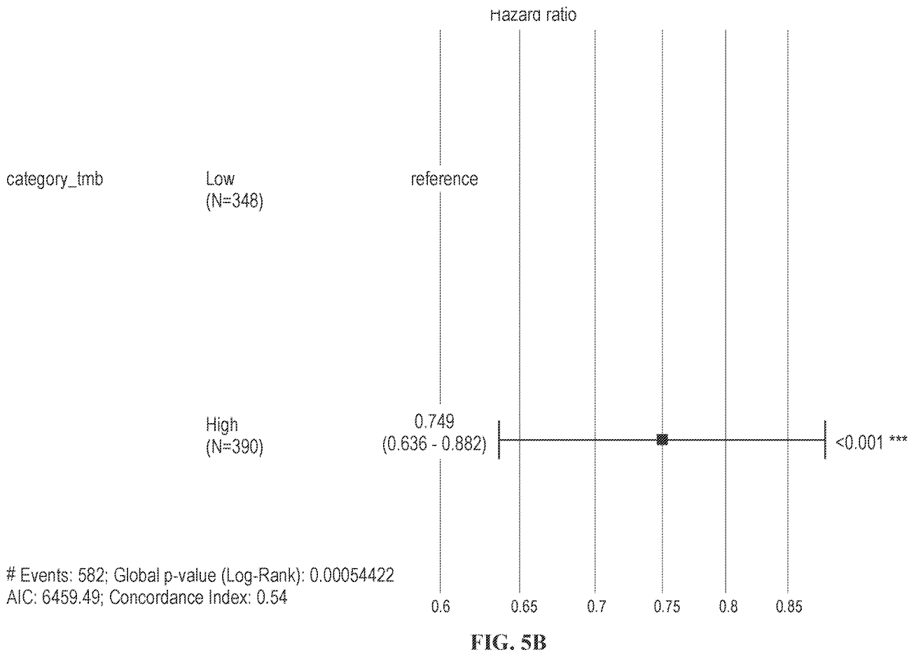

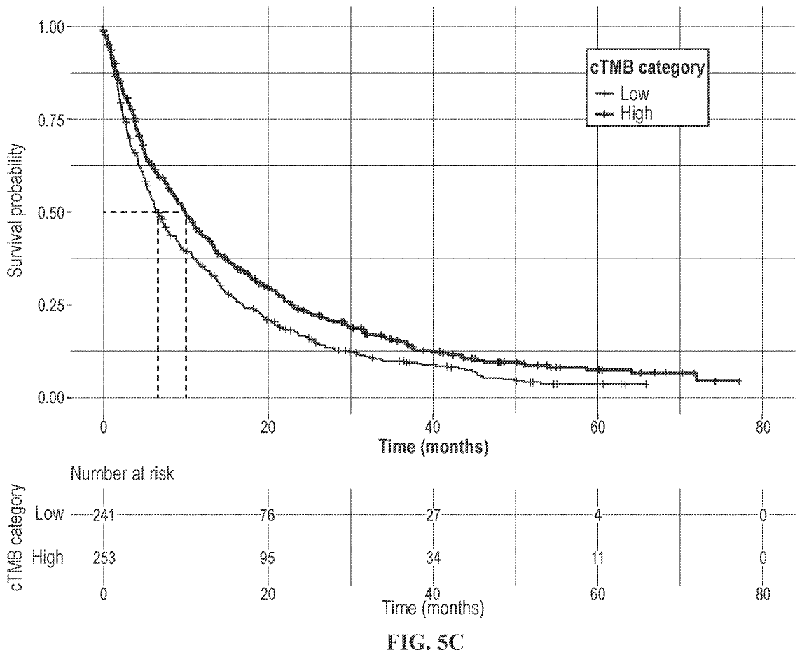

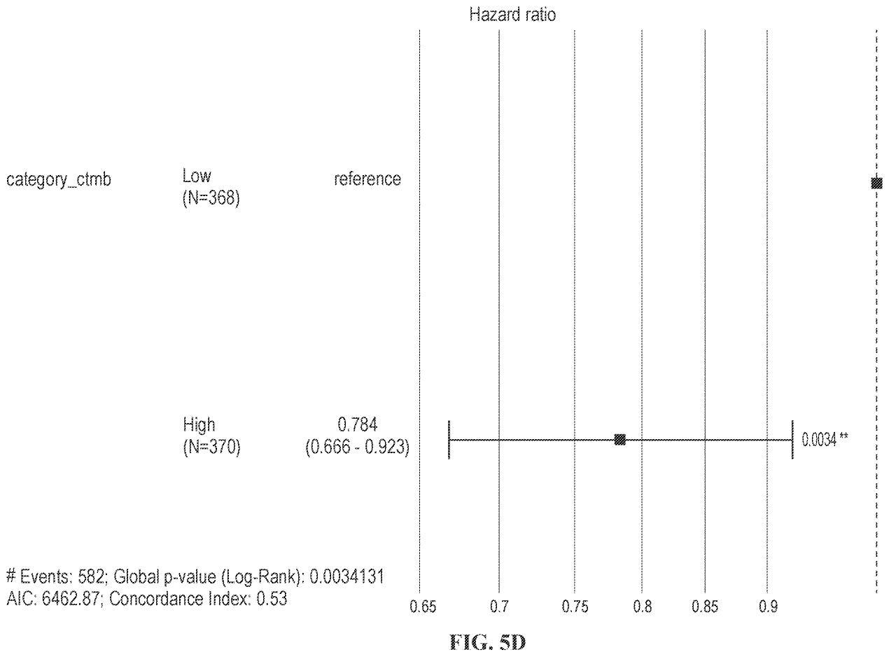

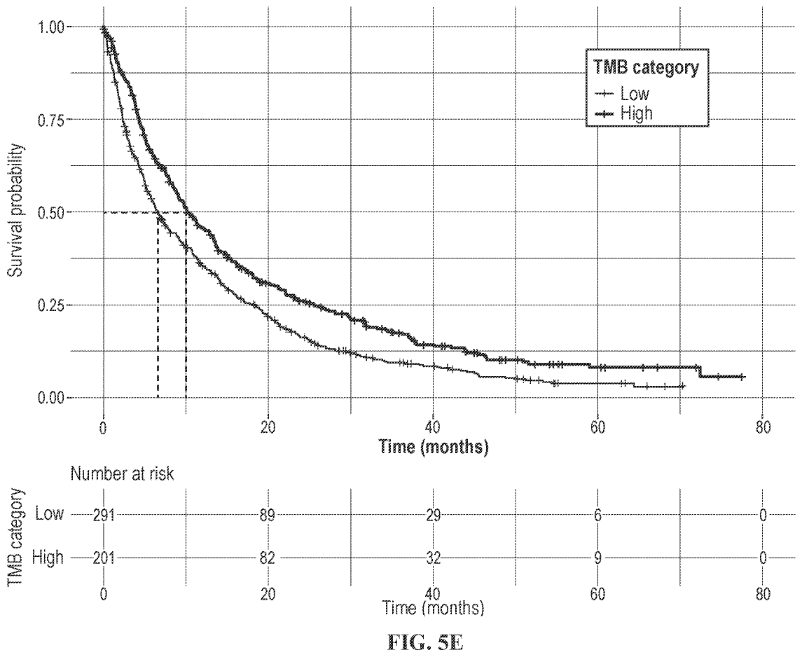

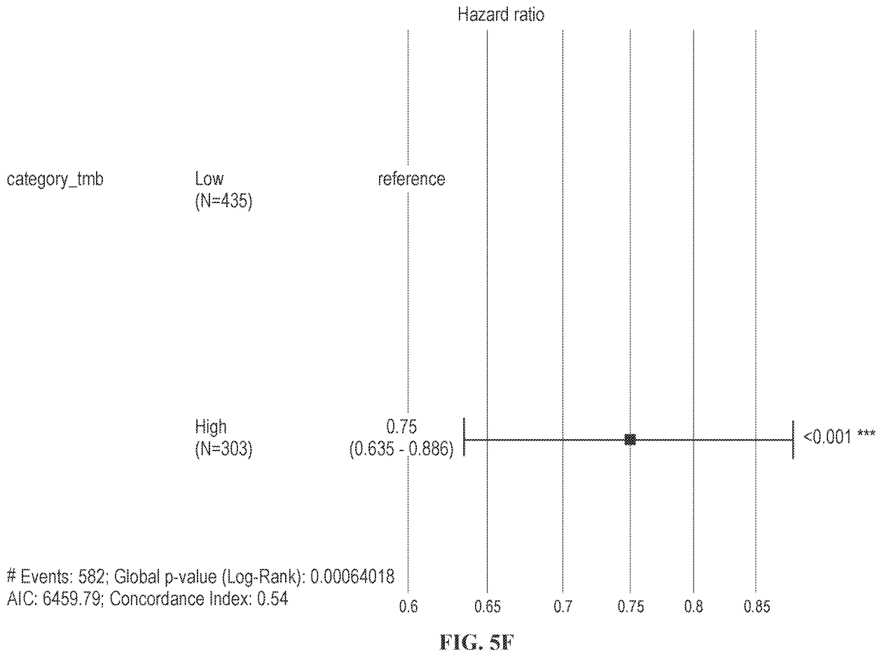

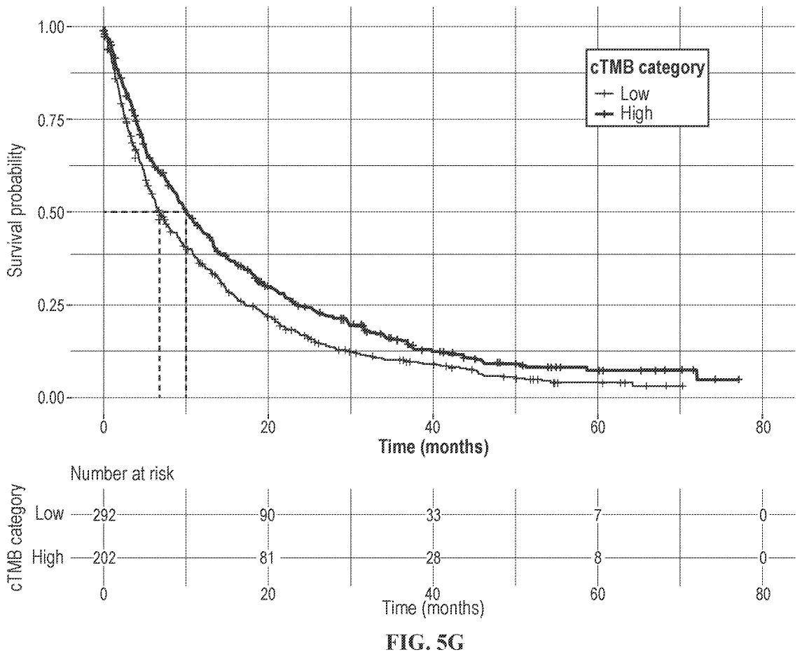

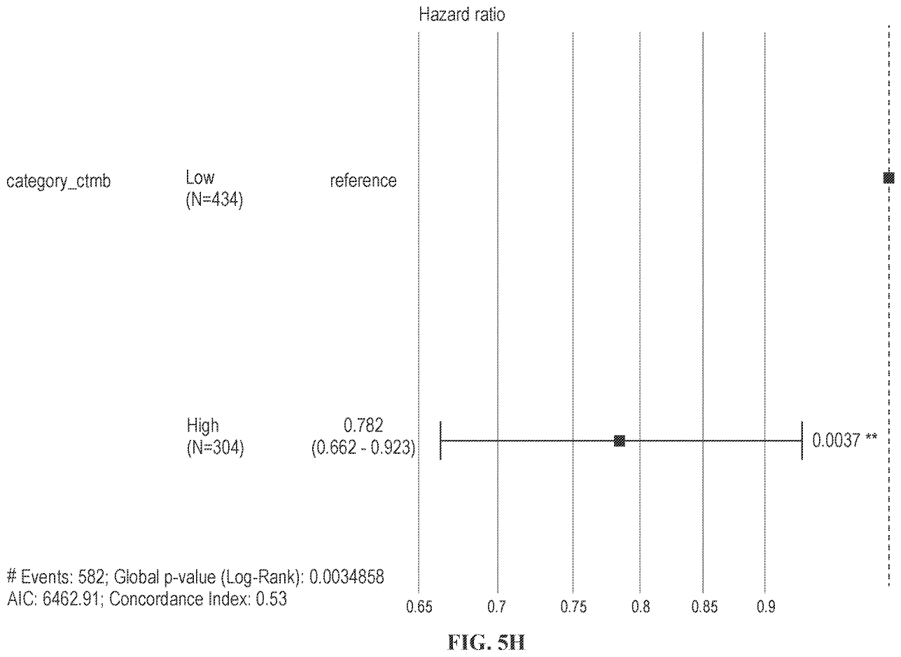

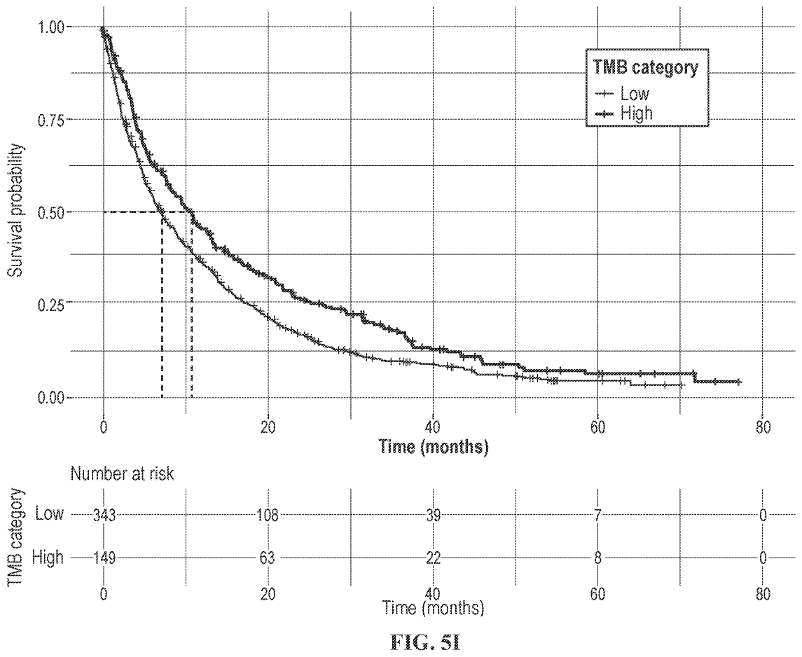

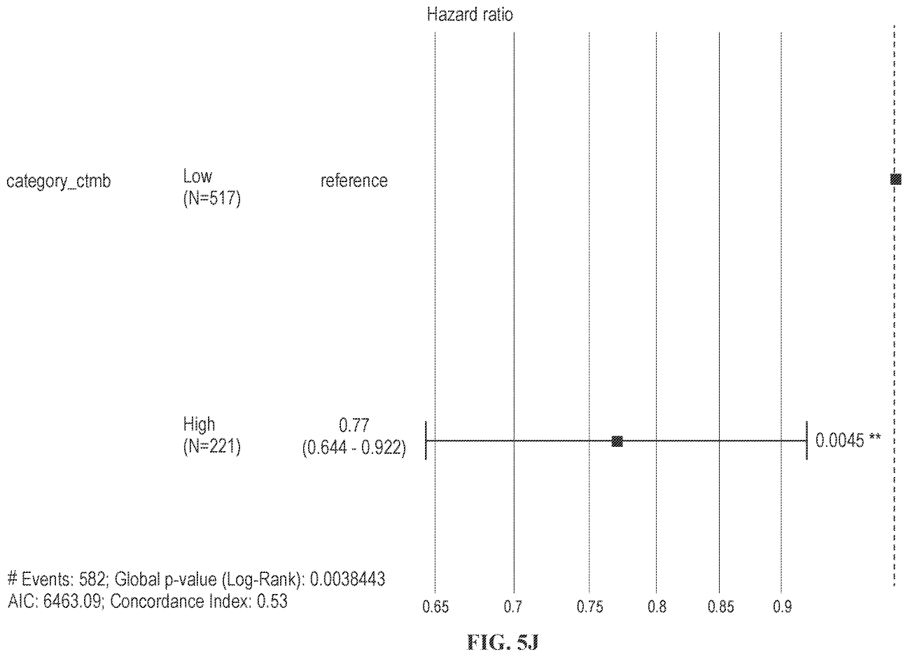

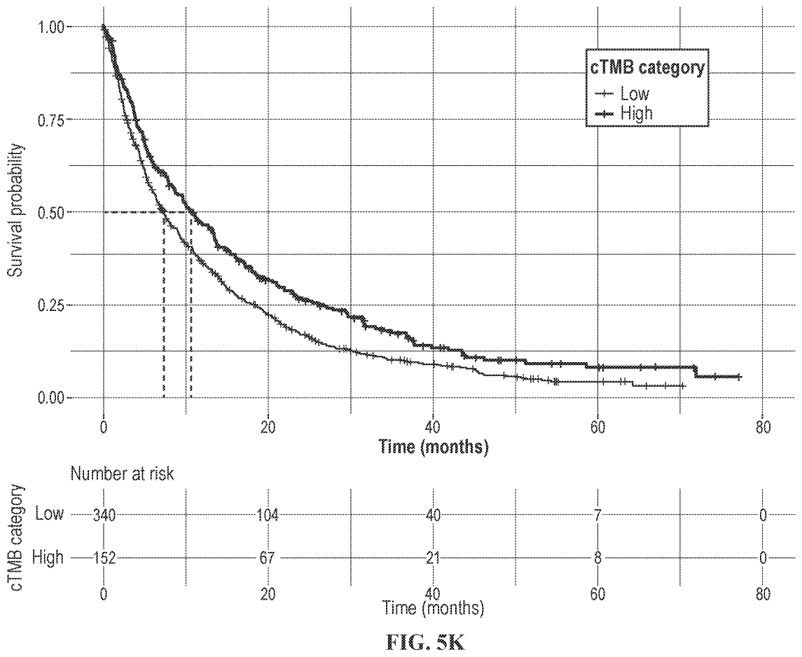

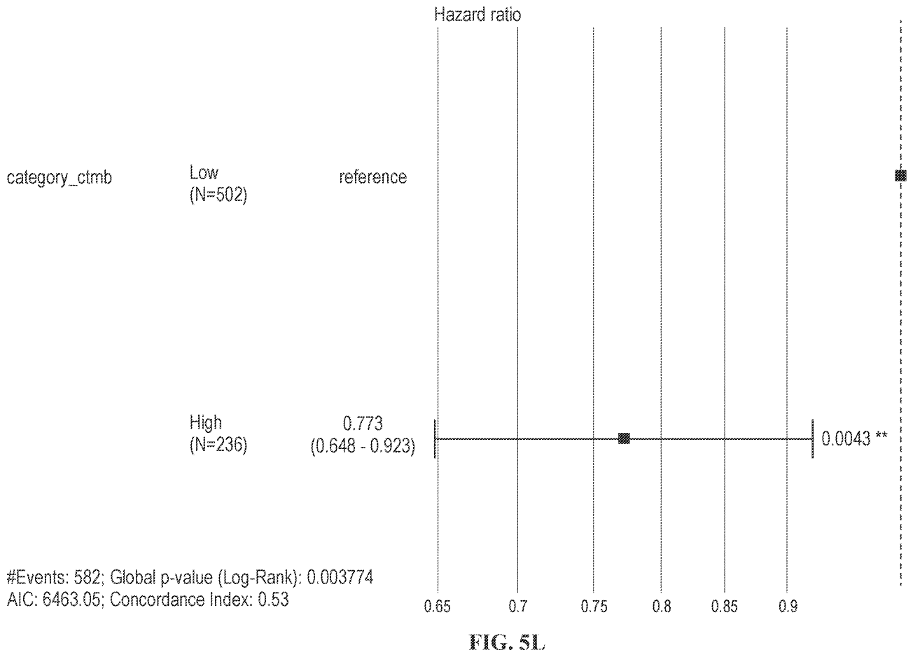

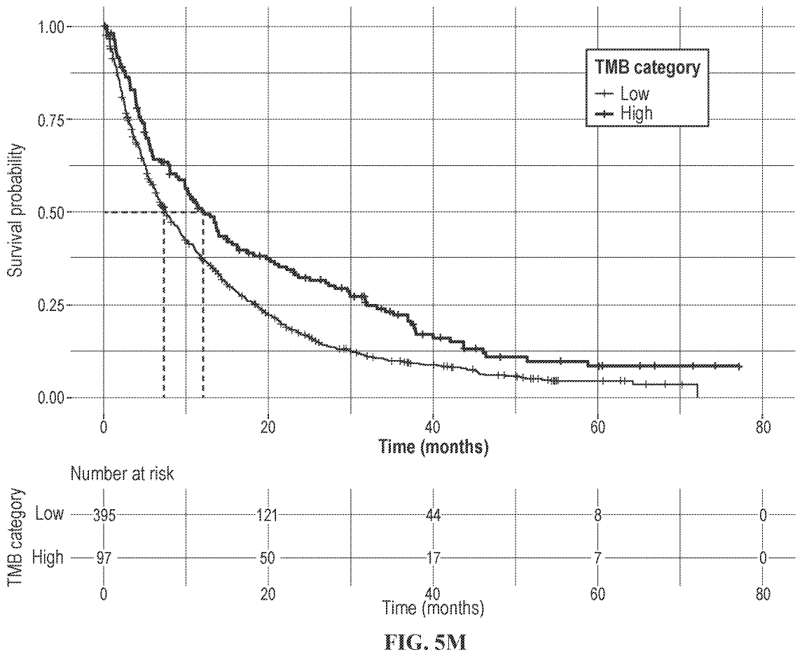

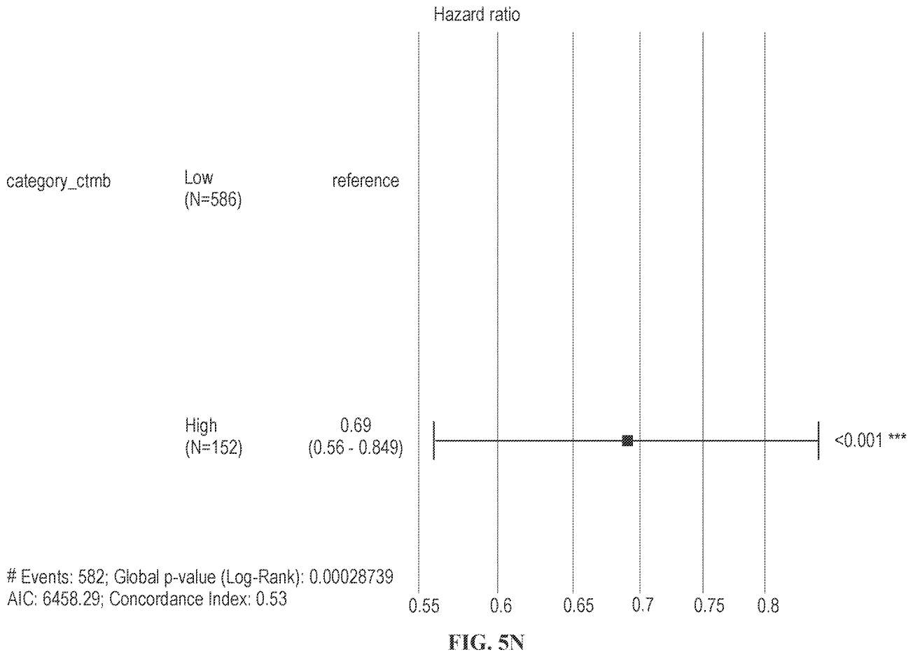

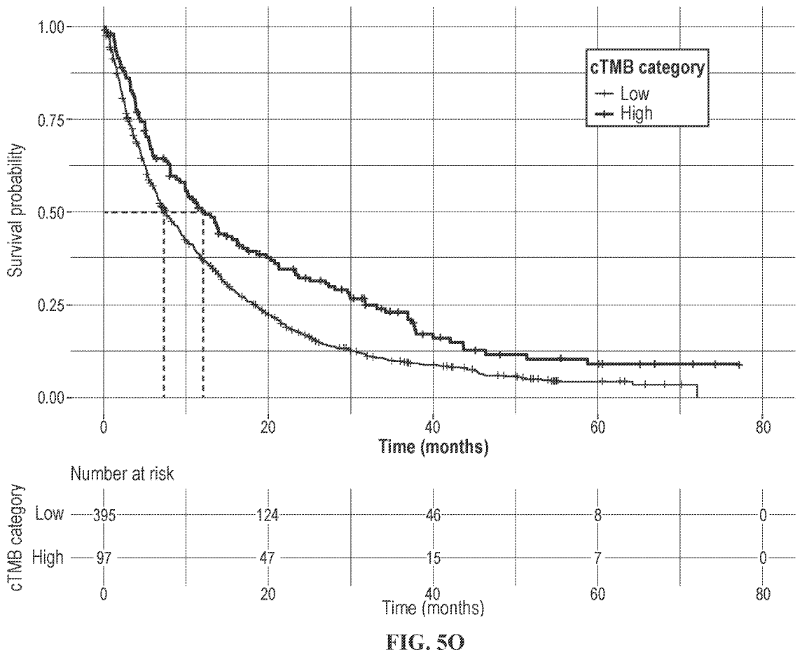

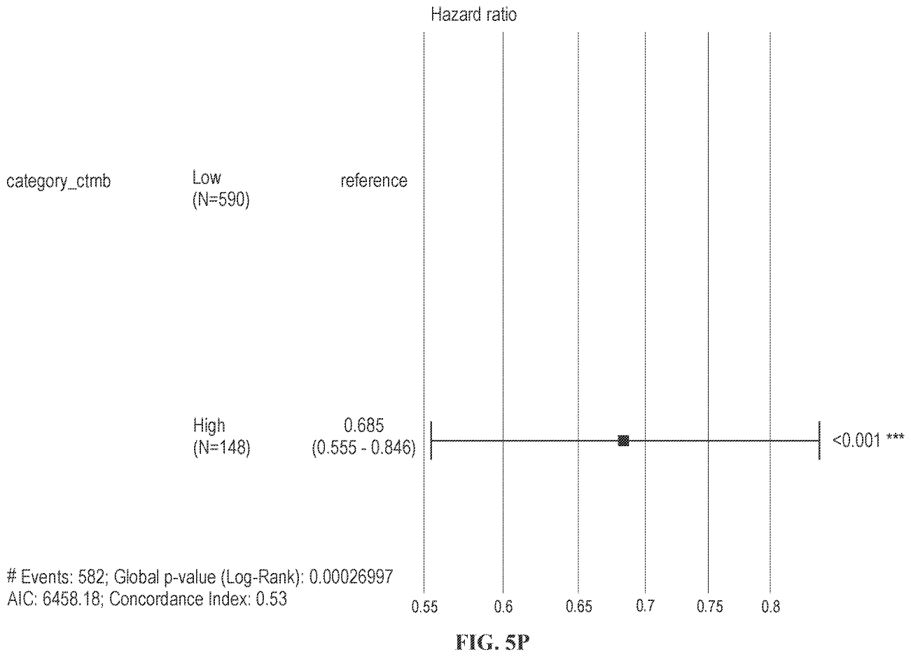

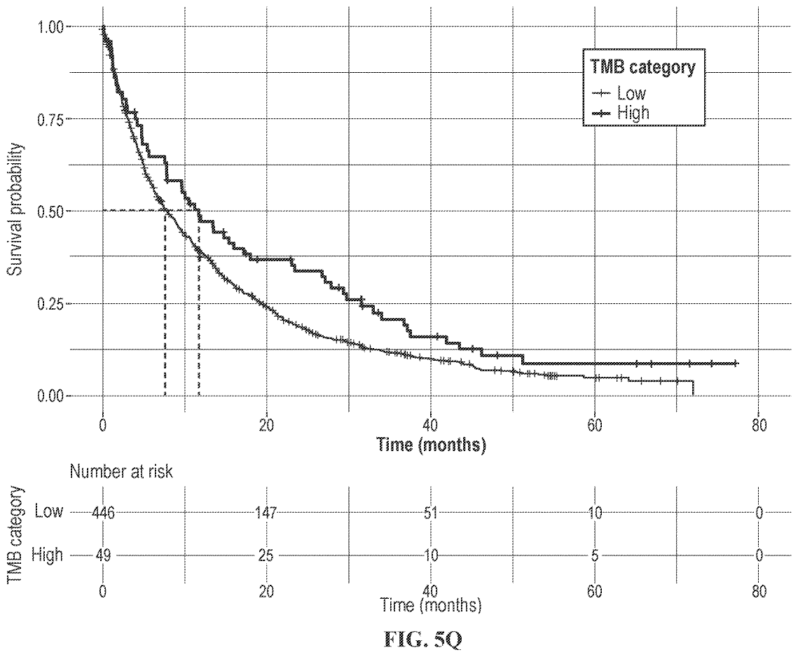

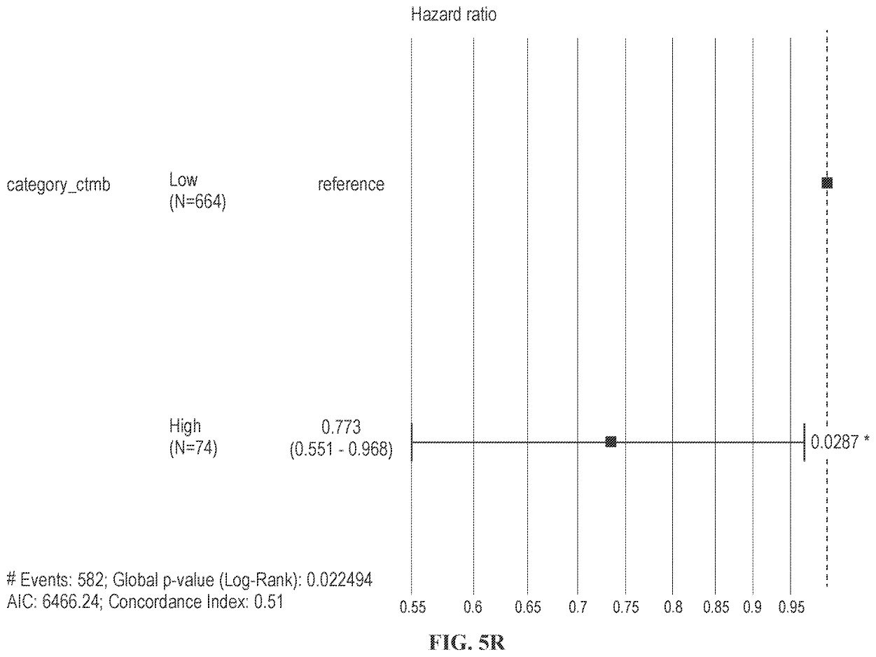

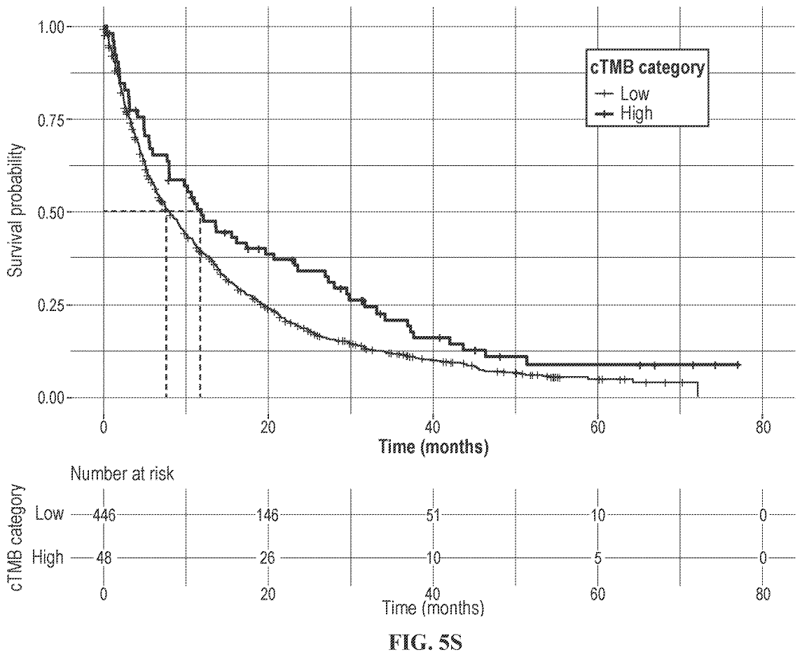

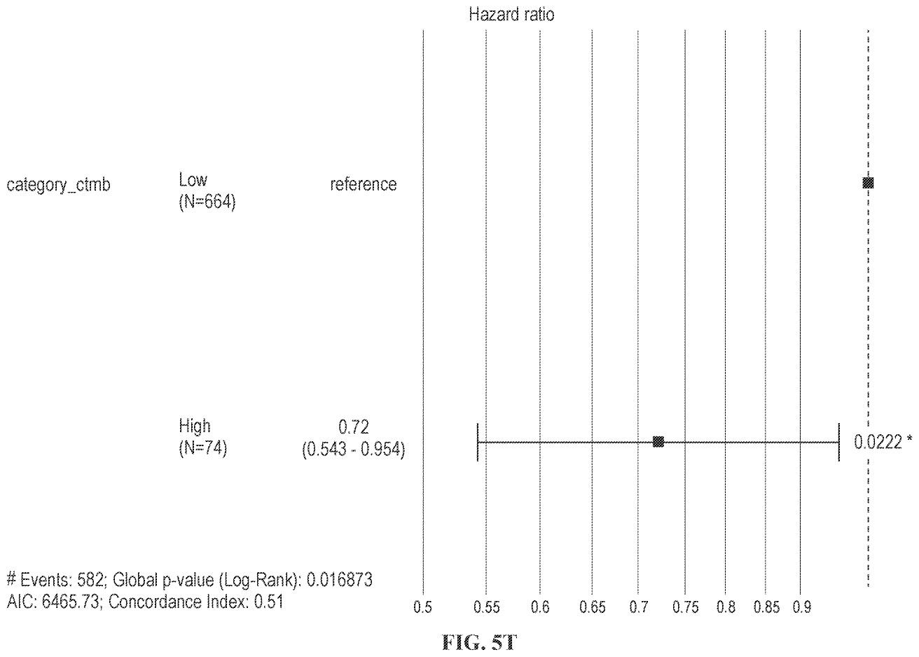

The 9048 specimen were identified from Part 1 were filtered in a parallel manner in Part 2 of the NSCLC CGDB analysis (FIG. 4). Here, specimen were removed if they failed the 90-day gap rule (N=2005), where the first clinical visit after metastatic diagnosis was after more than 90 days. Further, specimen were removed that had an advanced diagnosis date before a selected threshold date (N=91) or failed the clinical testing gap (N=497), where a comprehensive genomic profiling report was generated greater than 60 days after the last clinical visit date. From this, 6798 remaining specimens, EGFR wild-type and ALK wild-type specimens (N=5531) were sorted for first line immune checkpoint inhibitor (ICI) monotherapy (N=813) and second line ICI monotherapy (N=737). These specimen samples were then used in Line 2 Study (FIG. 5A-5T), where Univariate Kaplan Meier and Cox Proportional Hazards models were generated for a TMB and cTMB thresholds of various percentiles of TMB and cTMB respectively, of Line 2 Study. The Univariate Kaplan Meier plots showed overall survival (OS) probability versus time from start of second line immune checkpoint inhibitor (ICI) monotherapy treatment for the NSCLC patient cohort stratified by tumor mutational burden (TMB) or clonal TMB (cTMB) value, in accordance with some instances of the methods and systems described herein. Cox Proportional Hazards models showed results for a Cox proportional hazards regression model from start of second line immune checkpoint inhibitor (ICI) monotherapy treatment for the NSCLC patient cohort stratified by tumor mutational burden (TMB) or clonal TMB (cTMB) value, in accordance with some instances of the methods and systems described herein.

Results from the Line 2 Study for TMB and cTMB at different thresholds are shown in Table 1 and Table 2, respectively.

TABLE 1
Summary Of Clinical Outcomes in a Line 2 Immunotherapy
Treated Cohort At Different TMB Thresholds

| TMB | TMB | | | mOS | mOS | | | |
|---|---|---|---|---|---|---|---|---|
| threshold | threshold | N | N | High | Low | | | Concordance |
| percentile | value | high | low | (months) | (months) | HR | P Value | Index |
| 0.5 | 9.569952 | 387 | 343 | 10.03 | 6.18 | 0.74926 | 0.000524 | 0.54 |
| 0.6 | 11.30994 | 301 | 429 | 10.03 | 6.64 | 0.74965 | 0.000708 | 0.539 |
| 0.7 | 13.78569 | 220 | 510 | 10.88 | 7.27 | 0.77042 | 0.00449 | 0.532 |
| 0.8 | 16.52992 | 151 | 579 | 12.00 | 7.36 | 0.6899 | 0.00047 | 0.532 |
| 0.9 | 25.09428 | 74 | 656 | 11.74 | 7.76 | 0.7301 | 0.0287 | 0.513 |

TABLE 2
Summary Of Clinical Outcomes in a Line 2 Immunotherapy Treated
Cohort At Different clonal TMB (cTMB) Thresholds

| cTMB | cTMB | | | mOS | mOS | | | |
|---|---|---|---|---|---|---|---|---|
| threshold | threshold | N | N | High | Low | | | Concordance |
| percentile | value | high | low | (months) | (months) | HR | P Value | Index |
| 0.5 | 7.501163 | 367 | 363 | 9.96 | 6.64 | 0.7838 | 0.00341 | 0.533 |
| 0.6 | 8.751356 | 302 | 428 | 10.03 | 6.77 | 0.7815 | 0.00373 | 0.533 |
| 0.7 | 9.569952 | 234 | 496 | 10.52 | 7.27 | 0.7732 | 0.00434 | 0.531 |
| 0.8 | 13.88637 | 147 | 583 | 12.00 | 7.33 | 0.6852 | 0.000456 | 0.531 |
| 0.9 | 18.90771 | 74 | 656 | 11.74 | 6.64 | 0.7199 | 0.0222 | 0.515 |

### Conclusions

Collectively, these analyses and data support clonal tumor mutational burden (cTMB) as a superior predictor of mutation burden in tumor samples compared to TMB alone. Further, cTMB performs better for predicting immunotherapy response (e.g., response to immune checkpoint inhibitors (ICI)) compared to TMB alone.

### Example 2: Pan-Cancer Analysis of Tumor Mutational Burden (TMB) and Clonal TMB (cTMB)

This Example describes the pan-cancer landscape of tumor mutational burden (TMB) as compared to clonal TMB (cTMB).

Response to immune checkpoint inhibitors (ICI) relies on T-cell recognition of neoantigens on the surface of tumor cells. It is hypothesized that the clonality of a neoantigen determines the probability of its presentation to the immune system, with more clonal neoantigens having a higher likelihood of recognition. Using a TMB metric that is comprised of only clonal variants (cTMB) could refine TMB as a biomarker of ICI response.

### Methods

Tumor samples were assayed during routine clinical care, by hybrid capture-based comprehensive genomic profiling. TMB was defined as the number of non-driver somatic mutations identified on up to 1.1 megabases (Mb) of sequenced DNA. For every sample, TMB variants with a cancer cell fraction (CCF)<0.5% were filtered out to calculate the cTMB. Mutational signatures (sig) were assigned based on the COSMIC (Catalogue of somatic mutations in cancer) signatures.

### Results

The median TMB and median cTMB of the evaluable cohort (N=233,981) across 46 solid tumor types was 3.7 [1.3-6.7] and 2.7 [1.3-5.3] mutations per Mb (mut/Mb) respectively, with 59.1% of the samples having an identical TMB and cTMB (ITC). Distribution across major tumor types shown in Table 3.

TABLE 3
Distribution of TMB and cTMB across Major Tumor Types

| | | | | | ITC |
|---|---|---|---|---|---|
| Cancer | Sig | N | TMB | cTMB | (%) |
| NSCLC | Smoking | 5220 | 14.7[10.1-21.4] | 12.9[8.3-18.7] | 34.7 |
| | Smoking | 33127 | 5.5[2.7-10.1] | 4.6[2.7-8.3] | 50.8 |
| | wt | | | | |
| Colorectal | dMMR | 2021 | 10.7[6.4-41.4] | 8.3[5.3-36.1] | 26 |
| | dMMR wt | 28648 | 2.8[1.8-5.3] | 2.7[1.3-4.0] | 62.7 |
| Glioma | Alkylation | 127 | 65.5[39.8-106.9] | 40.1[17.4-62.6] | 7.1 |
| | Alkylation | 7595 | 1.8[0.9-2.8] | 1.3[0.9-2.8] | 73.4 |
| | wt | | | | |
| Melanoma | UV | 3394 | 21.2[12.0-38.8] | 20.1[12.0-36.8] | 55.1 |
| | UV wt | 3184 | 2.7[1.3-5.3] | 2.7[0.9-4.0] | 75.4 |
| Breast | | 29190 | 2.7[1.3-5.3] | 1.8[1.3-4.0] | 57.8 |
| Ovary | | 16314 | 2.7[1.3-4.0] | 1.8[0.9-2.8] | 58.7 |
| Pancreas | | 11995 | 1.8[1.3-2.8] | 1.3[0.9-2.7] | 75.8 |
| Prostate | | 11829 | 1.8[1.3-3.7] | 1.3[0.9-2.7] | 70.5 |
| Esophagus | | 7777 | 4.0[2.7-6.7] | 2.8[1.3-5.3] | 47.9 |

Smoking sig in NSCLC (non-small cell lung cancer; 34.7% vs 50.8%, P=8.9e-105), ultra-violet sig in melanoma (55.1% vs 75.4%, P=1.7e-67), mismatch repair sig in colorectal (26.0% vs 62.7%, P=1.5e-229) and alkylating sig in glioma (7.1% vs 73.4%, P=1.4e-55) had significantly lower ITC compared to their respective wild-type (healthy) samples. Local tumors had a comparable ITC to that of metastatic tumors (59.1% vs 58.9%).

Clonal tumor mutational burden (cTMB) is diverse across tumor types. As such, cTMB determined using the method described herein is an excellent predictor of ICI response pan-cancer, using tumor-type specific thresholds.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

What is claimed is:

1. A method comprising:
receiving, at one or more processors, genomic data for a subject, wherein the genomic data indicates a presence or absence of one or more short variants in a sample derived from the subject;
filtering, using the one or more processors, the genomic data to select, from the one or more short variants, one or more clonal tumor mutational burden (cTMB)-qualified short variants, wherein the filtering comprises excluding short variants identified as subclonal based on a tumor amount measure for a respective short variant; and
determining, using the one or more processors, a cTMB value for the sample based on the number of cTMB-qualified short variants.

2. The method of any of claim 1, wherein the subject has a cancer.

3. The method of claim 1, wherein the tumor amount measure comprises a cancer cell fraction (CCF) of the short variant or a variant allele frequency (VAF) for the short variant.

4. The method of claim 1, wherein the filtering comprises excluding short variants identified as having an allele frequency below a predetermined threshold.

5. The method of claim 1, wherein the filtering comprises excluding short variants identified as a germline variant.

6. The method of claim 1, wherein the filtering comprises excluding short variants identified as having a cancer cell fraction (CCF) at or below a predetermined CCF threshold.

7. The method of claim 1, wherein a clonal tumor mutational burden (cTMB) value is expressed as a function of the number of cTMB-qualified short variants per megabase (mutations/Mb) in a set of subgenomic intervals from the sample.

8. The method of claim 1, wherein the genomic data for the subject is based on a targeted exome sequencing panel.

9. The method of claim 1, wherein the genomic data for the subject is derived from circulating tumor DNA in a liquid biopsy sample.

10. The method of claim 9, wherein the sample is a liquid biopsy sample and comprises circulating tumor cells (CTCs).

11. The method of claim 9, wherein the sample is a liquid biopsy sample and comprises cell-free DNA (cfDNA), circulating tumor DNA (ctDNA), or any combination thereof.

12. The method of claim 1, wherein the genomic data is obtained by sequencing the sample.

13. The method of claim 9, wherein the sequencing comprises:
providing a plurality of nucleic acid molecules obtained from the sample from the subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the set of nucleic acid molecules in the sample.

14. A method of treating a subject having a cancer comprising:
(a) determining a clonal tumor mutational burden (cTMB) value in a sample from the subject by the method of claim 1; and
(b) treating the subject with an immuno-oncology (IO) therapy if the cTMB value determined for the sample is at or above a threshold cTMB value.

15. A method of selecting a treatment for a subject having a cancer, the method comprising determining a clonal tumor mutational burden (cTMB) value in a sample from the subject by the method of claim 1, wherein if the cTMB value determined for the sample is at or above a threshold cTMB value, the subject is identified as one who may benefit from treatment with an immune-oncology (IO) therapy.

16. A method of identifying a subject having a cancer for treatment with an immune-oncology (IO) therapy comprising:
(a) determining a clonal tumor mutational burden (cTMB) value in a sample from the subject by the method of claim 1; and
(b) identifying the subject for treatment with the IO therapy if the cTMB value determined for the sample is at or above a threshold cTMB value.

17. A method of identifying one or more treatment options for a subject having a cancer, the method comprising:
(a) determining a clonal tumor mutational burden (cTMB) value in a sample from the subject by the method of claim 1; and
(b) generating a report comprising one or more treatment options identified for the subject based at least in part on the cTMB value determined for the sample, wherein if the cTMB value is at or above a threshold cTMB value, the subject is identified as one who may benefit from treatment with an immune-oncology (IO) therapy.

18. A method of predicting survival of a subject having cancer, the method comprising determining a clonal tumor mutational burden (cTMB) value in a sample from the subject by the method of claim 1, wherein if the cTMB value determined for the sample is at or above a threshold cTMB value, the subject is predicted to have longer survival when treated with an immune-oncology (IO) therapy, as compared to a subject determined to have a cTMB value below the threshold cTMB value.

19. The method of claim 14, wherein the immuno-oncology (IO) therapy comprises an immune checkpoint inhibitor.

20. A system comprising:
one or more processors, and
a memory communicatively coupled to the one or more processors and configured to store instructions that, when executed by the one or more processors, cause the system to:
receive genomic data for a subject, wherein the genomic data indicates a presence or absence of one or more short variants in a sample derived from the subject;
filter the one or more short variants to select one or more clonal tumor mutational burden (cTMB)-qualified short variants, wherein the filtering comprises excluding short variants identified as subclonal based on a tumor amount measure for a respective short variant; and
determine a cTMB value for the sample based on the number of cTMB-qualified short variants.
